SEQUENCE LISTING (66 sequences)

SEQ ID NO: 1 - protein, 85 aa, synthetic construct
  source 1..85: note = Tetramerization domain from SEPPALLATA-like MADS domain transcription factor from Arabidopsis thaliana (SMDTF)
VELSSQQEYLKLKERYDALQRTQRNLLGEDLGPLSTKELESLERQLDSSLKQIRALRTQFMLDQSKERMLTETNKTLRLRLADGY

SEQ ID NO: 2 - protein, 113 aa, synthetic construct
  source 1..113: note = Sendai virus phosphoprotein tetramerization domain (SPP)
ENTSSMKEMATLLTSLGVIQSAQEFESSRDASYVFARRALKSANYAEMTFNVCGLILSAEKSSARKVDENKQLLKIQESVESFRDIYKRFSEYQKEQNSLLMSNLSTLHIITD

SEQ ID NO: 3 - protein, 24 aa, synthetic construct
  source 1..24: note = PiLZ structure from Xhantomonas campestris
LLVQRMDAKLDLILALIGRLVRQS

SEQ ID NO: 4 - protein, 74 aa, synthetic construct
  source 1..74: note = Measles virus phosphoprotein tetramerization domain (MPP)
GDHYDDELFSDVQDIKTALAKIHEDNQKIISKLESLLLLKGEVESIKKQINRQNISISTLEGHLSSIMIAIPGL

SEQ ID NO: 5 - protein, 51 aa, synthetic construct
  source 1..51: note = Dictyocaulus viviparus ACE tetramerization domain
AVADVGDPFLLWKQQMDKWQNEYITDWQYHFEQYKKYQTYRHLDSDSCSGS

SEQ ID NO: 6 - protein, 454 aa, synthetic construct
  source 1..454: note = PR8 N1 Wild-type
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 7 - protein, 454 aa, synthetic construct
  source 1..454: note = PR8 N1 I48C
MNPNQKITTIGSICLVVGLISLILQIGNIISIWISHSIQTGSQNHTGCCNQNIITYKNSTWVKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 8 - protein, 454 aa, synthetic construct
  source 1..454: note = PR8 N1 N50C
MNPNQKITTIGSICLVVGLISLILQIGNIISIWISHSIQTGSQNHTGICCQNIITYKNSTWVKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 9 - protein, 455 aa, synthetic construct
  source 1..455: note = PR8 N1 61insertC
MNPNQKITTIGSICLVVGLISLILQIGNIISIWISHSIQTGSQNHTGICNQNIITYKNSTWCVKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 10 - protein, 454 aa, synthetic construct
  source 1..454: note = PR8 N1 I48C_N50C
MNPNQKITTIGSICLVVGLISLILQIGNIISIWISHSIQTGSQNHTGCCCQNIITYKNSTWVKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 11 - protein, 455 aa, synthetic construct
  source 1..455: note = PR8 N1 I48C_61insertC
MNPNQKITTIGSICLVVGLISLILQIGNIISIWISHSIQTGSQNHTGCCNQNIITYKNSTWCVKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 12 - protein, 469 aa, synthetic construct
  source 1..469: note = HK14 N2 Wild-type
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 13 - protein, 469 aa, synthetic construct
  source 1..469: note = HK14 N2 L52C
MNPNQKIITIGSVSLTISTICFFMQIAILITTVTLHFKQYEFNSPPNNQVMCCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 14 - protein, 469 aa, synthetic construct
  source 1..469: note = HK14 N2 L52C_E54C
MNPNQKIITIGSVSLTISTICFFMQIAILITTVTLHFKQYEFNSPPNNQVMCCCPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 15 - protein, 481 aa, synthetic construct
  source 1..481: note = N1.2
MLLVNQSHQGFNKEHTSKMVSAIVLYVLLAAAAHSAFAADPHHHHHHSLVPRGSPSRIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 16 - protein, 481 aa, synthetic construct
  source 1..481: note = N1.3 (C49A - monomer)
MLLVNQSHQGFNKEHTSKMVSAIVLYVLLAAAAHSAFAADPHHHHHHSLVPRGSPSRIETANQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 17 - protein, 481 aa, synthetic construct
  source 1..481: note = N1.4 (T48C)
MLLVNQSHQGFNKEHTSKMVSAIVLYVLLAAAAHSAFAADPHHHHHHSLVPRGSPSRIECCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 18 - protein, 481 aa, synthetic construct
  source 1..481: note = N1.5 (N50C)
MLLVNQSHQGFNKEHTSKMVSAIVLYVLLAAAAHSAFAADPHHHHHHSLVPRGSPSRIETCCQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 19 - protein, 481 aa, synthetic construct
  source 1..481: note = N1.6 (T48C+N50C)
MLLVNQSHQGFNKEHTSKMVSAIVLYVLLAAAAHSAFAADPHHHHHHSLVPRGSPSRIECCCQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 20 - protein, 481 aa, synthetic construct
  source 1..481: note = N1.7 (A76C)
MLLVNQSHQGFNKEHTSKMVSAIVLYVLLAAAAHSAFAADPHHHHHHSLVPRGSPSRIETCNQSVITYENNTWVNQTYVNISNTNFACGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 21 - protein, 481 aa, synthetic construct
  source 1..481: note = N1.8 (Q78C)
MLLVNQSHQGFNKEHTSKMVSAIVLYVLLAAAAHSAFAADPHHHHHHSLVPRGSPSRIETCNQSVITYENNTWVNQTYVNISNTNFAAGCSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 22 - protein, 481 aa, synthetic construct
  source 1..481: note = N1.9 (V81C)
MLLVNQSHQGFNKEHTSKMVSAIVLYVLLAAAAHSAFAADPHHHHHHSLVPRGSPSRIETCNQSVITYENNTWVNQTYVNISNTNFAAGQSVCSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 23 - protein, 481 aa, synthetic construct
  source 1..481: note = N1.11 (W61C)
MLLVNQSHQGFNKEHTSKMVSAIVLYVLLAAAAHSAFAADPHHHHHHSLVPRGSPSRIETCNQSVITYENNTCVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 24 - protein, 530 aa, synthetic construct
  source 1..530: note = SMDTF-N1
MLLVNQSHQGFNKEHTSKMVSAIVLYVLLAAAAHSAFAADPHHHHHHVELSSQQEYLKLKERYDALQRTQRNLLGEDLGPLSTKELESLERQLDSSLKQIRALRTQFMLDQSKERMLTETNKTLRLRLADGYSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 25 - protein, 558 aa, synthetic construct
  source 1..558: note = SPP-N1
MLLVNQSHQGFNKEHTSKMVSAIVLYVLLAAAAHSAFAADPHHHHHHENTSSMKEMATLLTSLGVIQSAQEFESSRDASYVFARRALKSANYAEMTFNVCGLILSAEKSSARKVDENKQLLKIQESVESFRDIYKRFSEYQKEQNSLLMSNLSTLHIITDSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 26 - protein, 469 aa, synthetic construct
  source 1..469: note = PiLZ-N1
MLLVNQSHQGFNKEHTSKMVSAIVLYVLLAAAAHSAFAADPHHHHHHLLVQRMDAKLDLILALIGRLVRQSSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 27 - protein, 519 aa, synthetic construct
  source 1..519: note = MPP-N1
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 28 - protein, 496 aa, synthetic construct
  source 1..496: note = ACE-N1
MLLVNQSHQGFNKEHTSKMVSAIVLYVLLAAAAHSAFAADPHHHHHHAVADVGDPFLLWKQQMDKWQNEYITDWQYHFEQYKKYQTYRHLDSDSCSGSSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 29 - protein, 454 aa, synthetic construct; identical to SEQ ID NO: 6
  source 1..454: note = N1 PR8

SEQ ID NO: 30 - protein, 469 aa, synthetic construct
  source 1..469: note = N1_Mich15
MNPNQKIITIGSICMTIGMANLILQIGNIISIWVSHSIQIGNQSQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 31 - protein, 453 aa, synthetic construct
  source 1..453: note = N1_WSN
MNPNQKIITIGSICMVVGIISLILQIGNIISIWISHSIQTGNQNHTGICNQGSITYKVVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 32 - protein, 469 aa, synthetic construct
  source 1..469: note = N2_HK14
  SITE 148: note = X is unknown in public database
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNXVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 33 - protein, 469 aa, synthetic construct
  source 1..469: note = N2_ck
  SITE 356: note = X is unknown in public database
MNPNQKIIALGSASLTIAIVCLLIQIAILATTMTLHFTQSEYTNSSTNKVVSCESTIIERNITEIVHLNGTIIERESCPKSAEYKNWSKPQCQITGFAPFSKDNSIRLSAGGDIWVTREPYVSCSLNKCYQFALGQGTTLNNRHSNGTTHDRSPYRTLLMSELGVPFHLGTKQVCIAWSSSSCHDGRAWLHVCVTGDDKNATASVIYDGMLTDSIVSWSKNILRTQESECVCINGTCTVVMTDGSASGMADTRILFIREGKIVHISPLSGSAQHVEECSCYPRYPEVRCVCRDNWKGSNRPVLYINMADYNIDSNYVCSGLVGDTPRSDDSSSSSNCRDPNNERGGPGVKGWAFDXGDDVWMGRTIKKDSRAGYETFRVVDGWTVANSKSQTNRQVIVESDNRSGYSGIFSVEGKTCINRCFYVELIRGRPQETRVWWTSNSIIVFCGTSGTYGTGSWPDGANINFMSI

SEQ ID NO: 34 - protein, 469 aa, synthetic construct
  source 1..469: note = N3_sw
MNPNQKIITIGVVNTTLSTIALLIGVGNLIFNTVIHEKIGDHQIVTYPTITTPAVPNCSDTIITYNNTVINNITTTIITEEERPFKSPLPLCPFRGFFPFHKDNAIRLGENKDVIVTREPYVSCDNDNCWSFALTQGALLGTKHSNGTIKDRTPYRSLIRFPIGTAPVLGNYKEICIAWSSSSCFDGKEWMHVCMTGNDNDASAQIIYGGRMTDSIKSWRKDILRTQESECQCIDGTCVVAVTDGPAANSADYRVYWIREGKIIKYENVPKTKIQHLEECSCYVDIDVYCICRDNWKGSNRPWMRINNETILETGYVCSKFHSDTPRPADPSTMSCDSPSNVNGGPGVKGFGFKAGDDVWLGRTVSTSGRSGFEIIKVTEGWINSPNHVKSITQTLVSNNDWSGYSGSFIVKAKDCFQPCFYVELIRGRPNKNDDVSWTSNSIVTFCGLDNEPGSGNWPDGSNIGFMPK

SEQ ID NO: 35 - protein, 469 aa, synthetic construct
  source 1..469: note = N3_bwt
MNPNQKIITIGVVNTTLSTIALLIGVGNLVFNTVIHEKIGDHQTVTHPTITTPAIPNCSDTIITYNNTVINNITTTIITEAERPFKSPLPLCPFRGFFPFHKDNAIRLGENKGVIVTREPYISCDNDNCWSFALAQGALLGTKHSNGTIKDRTPYRSLIRFPIGTAPVLGNYKEICIAWSSSSCFDGKEWMHVCMTGNDNDASAQIIYAGRMTDSIKSWRKDILRTQESECQCIDGTCVVAVTDGPAANSADHRVYWIREGKIIKYEDVPKTKIQHLEECSCYVDIDVYCICRDNWKGSNRPWMRINNETILETGYVCSKFHSDTPRPADPSTMSCDSPSNVNGGPGVKGFGFKAGNDVWLGRTVSTSGRSGFEIIKVTEGWINSPNHAKSITQTLVSNNDWSGYSGSFIVKTKDCFQPCFYVELIRGRPNKNDDVSWTSNSIVTFCGLDNEPGSGNWPDGSNIGFMPK

SEQ ID NO: 36 - protein, 469 aa, synthetic construct
  source 1..469: note = N3_duck
MNPNQKIITIGVVNTTLSTIALLIGIGNLIFNTVIHEKIGVHQTVVYPTITPPVIPNCSDTTITYNNTVVNNITATIINKAEKQFKPSLPLCPFRGFFPFHKDNAIRLGENKDVIVTREPYVSCDNDDCWSFALAQGALLGTKHSNGTIKDRTPYRSLIRFPIGTAPVLGNYKEICVAWSSSSCFDGKEWMHVCMTGNDNDASGQIIYAGKMTDSIKSWRRDILRTQESECQCIDGTCIVAVTDGPAASSADHRIYWIRKGKIIKYEDIPKTKIQHLEECSCYVDTDVYCICRDNWKGSNRPWMRINNETILETGYVCSKFHSDTPRPADPSTVSCDSPSNINGGPGVKGFGFRAGNDVWLGRTVSTTGRSGFEVIKVTEGWINSLNHAKSITQTLVSNNDWSGYSGSFIVENNGCFQPCFYIELIRGRPNRNDDVSWTSNSIVTFCGLDNEPGSGNWPDGSNIGFMPK

SEQ ID NO: 37 - protein, 470 aa, synthetic construct
  source 1..470: note = N4_duck
MNPNQKIITIGSVSIVLTTIGLLLQITSLCSIWFSHYNQVTQTSEQSCSNNTTNYYNETFVNVTNVQNNYTTITEPSSPQVIHYSSGRDLCPVKGWAPLSKDNGIRIGSRGEVFVIREPFISCSINECRTFFLTQGALLNDKHSNGTVKDRSPFRTLMSCPIGVAPSPSNSRFESVAWSATACSDGPGWLTLGITGPDSTAVAVIKYNGIITDTLKSWKGNIMRTQESECVCQDEFCYTLITDGPSDAQAFYKILKIRKGKIMSVKDVDATGFHFEECSCYPSGENVECVCRDNWRGSNRPWIRFNSDLDYQIGYVCSGVFGDNPRPVDGTGSCSGPINNGKGRYGVKGFSFRYGDGVWIGRTKSLESRSGFEMVWDANGWVSTDKDSNGVQDIIDNDNWSGYSGSFSIRGETTGKNCTVPCFWVEMIRGQPKEKTIWTSGSSIAFCGVDSDTTGWSWPDGALLPFDIDK

SEQ ID NO: 38 - protein, 470 aa, synthetic construct
  source 1..470: note = N4_rt
MNPNQKIITIGSVSIILTTIGLLLQITSLCSIWFSHYNQVTQTHEQPCSNNTTNYYNETFVNVTNVQNNYTTVTEPSTPDVVHYSSGRDLCPIRGWAPLSKDNGIRIGSRGEVFVIREPFISCSISECRTFFLTQGALLNDKHSNGTVKDRSPFRTLMSCPIGVAPSPSNSRFESVAWSATACSDGPGWLTLGITGPDTTAVAVLKYNGIITDTFKSWKGNIMRTQESECVCQDEFCYTLITDGPSDAQAFYKILKIRKGKIVSMKDVDATGFHFEECSCYPSRTDIECVCRDNWRGSNRPWIRFNSDLDYQIGYVCSGIFGDNPRPVDGTGSCNSPVNNGKGRYGVKGFSFRYGDGVWIGRTKSLEFRSGFEMVWDANGWVSTDKDSNGVQDIIDNDNWSGYSGSFSIRGEKTGRNCTVPCFWVEMIRGQPKEKTIWTSGSSIAFCGVNSDTTGWSWPDGALLPFDIDK

SEQ ID NO: 39 - protein, 473 aa, synthetic construct
  source 1..473: note = N5_aw
MNPNQKIITIGSVSLALVVFNILLHIASIVIGIISVTKEISVSSTCNTTEVYNETVRLETITIPINNTVYIERESHQEPEFLNNTEPLCNVSGFAIVSKDNGIRIGSRGHVFVIREPFVACGPTECRTFFLTQGALLNDKHSNNTVKDRSPYRALMSVPLGSSPNAYQAKFESVAWSATACHDGKGWLAVGISGADDDAYAVIHYGGMPTDVVRSWRKQILRTQESSCVCMTGNCYWVMTDGPANSQASYKIFKSHRGMVTNEREVSFQGGHIEECSCYPNLGKVECVCRDNWNGMNRPVLTFDEDLNYEVGYLCAGIPTDTPRVQDNSFTGSCTNAVGGSGTNNYGVKGFGFRQGNSVWAGRTVSISSRSGFEILLIEDGWIKTSKNVVKKVEVLNNKNWSGYSGAFTIPITMTSKQCLVPCFWLEMIRGKPEERTSIWTSSSSTVFCGVSSEVPGWSWDDGAILPFDIDKM

SEQ ID NO: 40 - protein, 472 aa, synthetic construct
  source 1..472: note = N5_md
MNPNQKIITIGSISLGLVVFNILLHVASIVLGIISVTKDHGAYTCNTTEVYNETVRVETVTIPVNNTIYIERELTHEPEFLNNTEPLCEVSGFAIVSKDNGIRIGSRGHVFVIREPFVACGPSECRTFFLTQGALLNDKHSNNTVKDRSPYRALMSVPLGSSPNAYQAKFESVGWSATACHDGKEWMAIGVSGADDDAYAVIHYGGIPTDVVRSWRKQILRTQESSCVCMKGECYWVMTDGPANNQASYKIFKSQKGLVVDEKEISFQGGHIEECSCYPNMGKVECVCRDNWNGMNRPILTFDENLEYEVGYLCAGIPTDTPRVQDSSFTGSCTNAVGGSGTNNYGVKGFGFRQGTSVWAGRTISISSRSGFEVLLIKDGWIRPSKTISKKVEVLDNKNWSGYSGSFTIPTAMTSKSCLVPCFWLEMIRGKPEERTSIWTSSSSTVFCGVSSEVPGWSWDDGAILPFDIDKM

SEQ ID NO: 41 - protein, 459 aa, synthetic construct
  source 1..459: note = N6_Sz
MNPNQKITCISATGVTLSVVSLLIGIANLGLNIGLHYKVSDSTTINIPNMNETNSTTTNITNIIVNKNEERTFLNLTKPLCEVNSWHILSKDNAIRIGEDAHILVTREPYLSCDPQGCRMFALSQGTTLRGRHANGTIHDRGPFRALISWEMGQAPSPYNTRVECIGWSSTSCHDGISRMSICISGPNDNASAVVWYRGRPVTEIPSWVGNILRTQESECVCHKGICPVVMTDGPANNKAATKIIYFKEGKIQKIEELQGNAQHIEECSCYGAAGMIKCVCRDNWKGANRPIITIDPEMMTHTSKYLCSKILTDTSRPNDPTNGNCDAPITGGSPDPGVKGFAFLDGENSWLGRTISKDSRSGYEMLKVPNAETDTQSGPTSYQLIVNNQNWSGYSGAFIDYWANKGCFNPCFYVELIRGRPKEIDVLWTSSSMVALCGSRERLGSWSWHDGAEIIYFK

SEQ ID NO: 42 - protein, 470 aa, synthetic construct
  source 1..470: note = N6_cs
MNPNQKIICISATGMTLSVVSLLIGIANLGLNIGLHYKVGDTPDVNIPSMNETNSTTTIINNNTQNNFTNITNIIVNKEEGRTFLNLTKPLCEVNSWHILSKDNAIRIGEDAHILVTREPYLSCDPQGCRMFALSQGTTLRGRHANGTIHDRSPFRALVSWEMGQAPSPYNVRVECIGWSSTSCHDGISRMSICMSGPNNNASAVVWYGGRPVTEIPSWAGNILRTQESECVCHKGICPVVMTDGPANNRAATKIIYFKEGKIQKIEELEGNAQHIEECSCYGAAGVIKCICRDNWKGANRPVIIIDPEMMTHTSKYLCSRVLTDTSRPNDPTSGNCDAPITGGSPDPGVKGFAFLDGENSWLGRTISKDSRSGYEMLKVPNAETDTQSGPTSHQVIVNNQNWSGYSGAFIDYWANKECFNPCFYVELIRGRPKESSVLWTSNSIVALCGSKERLGSWSWHDGAEIIYFK

SEQ ID NO: 43 - protein, 471 aa, synthetic construct
  source 1..471: note = N7_cN
MNPNQKLFALSGVAIALSVLNLLIGISNVGLNVSLHLKEKGPKQEENLTCTTINQNNTTVVENTYVNNTTIITKGTDLKTPSYLLLNKSLCNVEGWVVIAKDNAVRFGESEQIIVTREPYVSCDPTGCKMYALHQGTTIRNKHSNGTIHDRTAFRGLISTPLGTPPTVSNSDFMCVGWSSTTCHDGIARMTICIQGNNDNATATVYYNRRLTTTIKTWARNILRTQESECVCHNGTCAVVMTDGSASSQAYTKVMYFHKGLVVKEEELRGSARHIEECSCYGHNQKVTCVCRDNWQGANRPIIEIDMNTLEHTSRYVCTGILTDTSRPGDKSSGDCSNPITGSPGAPGVKGFGFLNGDNTWLGRTISPRSRSGFEMLKIPNAGTDPNSRIAERQEIVDNNNWSGYSGSFIDYWNDNSECYNPCFYVELIRGRPEEAKYVWWTSNSLIALCGSPFPVGPGSFPDGAQIQYFS

SEQ ID NO: 44 - protein, 471 aa, synthetic construct
  source 1..471: note = N7_cG
MNPNQKLFALSGVAIALSVLNLLIGISNVGLNVSLHLKEKGPKQEENLTCTTINQNNTTVVENTYVNNTTIITKGTDLKTPSYLLLNKSLCNVEGWVVIAKDNAVRFGESEQIIVTREPYVSCDPTGCKMYALHQGTTIRNKHSNGTIHDRTAFRGLISTPLGTPPTVSNSDFMCVGWSSTTCHDGIARMTICIQGNNDNATATVYYNRRLTTTIKTWARNILRTQESECVCHNGTCAVVMTDGSASSQAYTKVMYFHKGLVVKEEELRGSARHIEECSCYGHNQKVTCVCRDNWQGANRPIIEIDMNTLEHTSRYVCTGILTDTSRPGDKSSGDCSNPITGSPGAPGVKGFGFLNGDNTWLGRTISPRSRSGFEMLKIPNAGTDPNSRIAERQEIVDNNNWSGYSGSFIDYWNDNSECYNPCFYVELIRGRPEEAKYVWWASNSLIALCGSPFPVGSGSFPDGAQIQYFS

SEQ ID NO: 45 - protein, 470 aa, synthetic construct
  source 1..470: note = N8_cN
MNPNQKIVTIGSISLGLVVFNVLLHAVSIILTVLALGKSENNGICNGTVVREYNETVRIEKVTQWYNTSVVEYVPHWNEGTYINNTEPICDVKGFAPFSKDNGIRVGSRGHIFVIREPFVSCSPVECRTFFLTQGSLLNDKHSNGTVKDRSPFRTLMSVEVGQSPNVYQARFEAVAWSATACHDGKKWMTIGVTGPDSKAVAVVHYGGVPTDVVNSWAGDILRTQESSCTCIQGNCYWVMTDGPANRQAQYRIYKANQGKIIGRTDVSFSGGHIEECSCYPNDGKVECVCRDNWTGTNRPVLIISPDLSYRVGYLCAGLPSDTPRGEDTQFAGSCTSPMGNQGYGVKGFGFRQGTDVWVGRTISRTSRSGFEIIRIKNGWTQTSKEQIRRQVVVDNSNWSGYSGSFTLPVELSGRECLVPCFWVEMIRGRPEERTIWTSSSSIVMCGVDYEIADWSWHDGAILPFDIDKT

SEQ ID NO: 46 - protein, 470 aa, synthetic construct
  source 1..470: note = N8_cL
MNPNQKIITIGSVSLGLVVLNILLHIVSITVTVLVLPRNGNNGSCNETVIREYNETVRVEKVTQWHNTNVIEYIERPENDHFMNNTEALCDAKGFAPFSKDNGIRIGSRGHVFVIREPFVSCSPTECRTFFLTQGSLLNDKHSNGTVKDRSPYRTLMSVEIGQSPNVYQARFEAVAWSATACHDGKKWMTIGVTGPDAKAVAVVHYGGIPTDVINSWAGDILRTQESSCTCIQGECFWVMTDGPANRQAQYRAFKAKQGKIVGQAEISFNGGHIEECSCYPNEGKVECVCRDNWTGTNRPVLVISPDLSYRVGYLCAGIPSDTPRGEDSQFTGSCTSPMGNQGYGVKGFGFRQGSDVWMGRTISRTSRSGFEILKVRNGWVQNSKEQIKRQVVVDNLNWSGYSGSFTLPVELTKRNCLVPCFWVEMIRGKPEEKTIWTSSSSIVMCGVDHEIADWSWHDGAILPFDIDKM

SEQ ID NO: 47 - protein, 465 aa, synthetic construct
  source 1..465: note = N9_AH13
MNPNQKILCTSATAIIIGAIAVLIGIANLGLNIGLHLKPGCNCSHSQPETTNTSQTIINNYYNETNITNIQMEERTSRNFNNLTKGLCTINSWHIYGKDNAVRIGESSDVLVTREPYVSCDPDECRFYALSQGTTIRGKHSNGTIHDRSQYRALISWPLSSPPTVYNSRVECIGWSSTSCHDGKSRMSICISGPNNNASAVVWYNRRPVAEINTWARNILRTQESECVCHNGVCPVVFTDGSATGPADTRIYYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAEGDCYRACFYVELIRGRPKEDKVWWTSNSIVSMCSSTEFLGQWNWPDGAKIEYFL

SEQ ID NO: 48 - protein, 465 aa, synthetic construct
  source 1..465: note = N9_ck
MNPNQKILCTSATAIIIGAIAVLIGIANLGLNIGLHLKPGCNCSHSQPETTNTSQTIINNYYNETNITNIQMEERTSKNFNNLTKGLCTINSWHIYGKDNAVRIGESSDVLVTREPYVSCDPDECRFYALSQGTTIRGKHSNGTIHDRSQYRALISWPLSSPPTVYNSRVECIGWSSTSCHDGKSRMSICISGPNNNASAVVWYNRRPVAEINTWARNILRTQESECVCHNGVCPVVFTDGSATGPADTRIYYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAKGDCYRACFYVELIRGRPKEDKVWWTSNSIVSMCSSTEFLGQWNWPDGAKIEYFL

SEQ ID NO: 49 - protein, 466 aa, synthetic construct
  source 1..466: note = B_Mal
MLPSTIQTLTLFLTSGGVLLSLYVSASLSYLPYSDILLKFPSTEITAPTMPLDCANASNVQAVNRSATKGVTLLLPEPEWTYPRLSCPGSTFQKALLISPHRFGETKGNSAPLIIREPFIACGPKECKHFALTHYAAQPGGYYNGTRGDRNKLRHLISVKLGKIPTVENSIFHMAAWSGSACHDGKEWTYIGVDGPDNNALLKIKYGEAYTDTYHSYANNILRTQESACNCIGGNCYLMITDGSASGVSECRFLKIREGRIIKEIFPTGRIKHTEECTCGFASNKTIECACRDNSYTAKRPFVKLNVETDTAEIRLMCTETYLDTPRPDDGSITGPCESNGDKGSGGIKGGFVHQRMASKIGRWYSRTMSKTKRMGMGLYVKYDGDPWADSDALAFSGVMVSMEEPGWYSFGFEIKDKKCDVPCIGIEMVHDGGKETWHSAATAIYCLMGSGQLLWDTVTGVNMAL

SEQ ID NO: 50 - protein, 6 aa, synthetic construct
  source 1..6: note = His tag
HHHHHH

SEQ ID NO: 51 - protein, 7 aa, synthetic construct
  source 1..7: note = Cleavage site
LVPRGSP

SEQ ID NO: 52 - protein, 7 aa, synthetic construct
  source 1..7: note = Cleavage site
  VARIANT 7: note = X is G or S
ENLYFQX

SEQ ID NO: 53 - protein, 102 aa, synthetic construct
  source 1..102: note = PR8 N1 (aa 1-102)
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVI

SEQ ID NO: 54 - protein, 117 aa, synthetic construct
  source 1..117: note = HK14 N2 (aa 1-117)
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVT

SEQ ID NO: 55 - DNA, 1539 bp, synthetic construct
  source 1..1539: mol_type = other DNA; note = N1-MPP (with his tag)
atgctgctcgtcaaccaatcccaccagggcttcaacaaggaacacacttctaagatggtctccgctatcgtgctctacgtgctgctcgctgccgctgcccactcagctttcgctgccgacccacaccaccaccaccaccacggcgatcactacgacgacgaactgttctccgacgtgcaggacatcaagaccgctctggctaagatccacgaggacaaccagaagatcatctccaagctggaatccctgctgctgctgaagggcgaagtcgagtccatcaagaagcagatcaaccgccagaacatctccatctccaccttggagggtcacctgtcctccatcatgatcgctatccctggcctgggcggcggctccgtgaaattagcgggcaattcctctctctgccctgttagtggatgggctatatacagtaaagacaacagtgtaagaatcggttccaagggggatgtgtttgtcataagggaaccattcatatcatgctctcccttggaatgcagaaccttcttcttgactcaaggggccttgctaaatgacaaacattccaatggaaccattaaagacaggagcccataccgaaccctaatgagctgtcctattggtgaagttccctctccatacaactcaagatttgagtcagtcgcttggtcagcaagtgcttgtcatgatggcatcaattggctaacaattggaatttctggcccagacagtggggcagtggctgtgttaaagtacaatggcataataacagacactatcaagagttggaggaacaatatattgagaacacaagagtctgaatgtgcatgtgtaaatggttcttgctttaccataatgaccgatggaccaagtgatggacaggcctcatacaaaatcttcagaatagaaaagggaaagataatcaaatcagtcgaaatgaaagcccctaattatcactatgaggaatgctcctgttaccctgattctagtgaaatcacatgtgtgtgcagggataactggcatggctcgaatcgaccgtgggtgtctttcaaccagaatctggaatatcagatgggatacatatgcagtggggttttcggagacaatccacgccctaatgataagacaggcagttgtggtccagtatcgtctaatggagcaaatggagtaaaaggattttcattcaaatacggcaatggtgtttggatagggagaactaaaagcattagttcaagaaaaggttttgagatgatttgggatccgaatggatggactgggactgacaataaattctcaataaagcaagatatcgtaggaataaatgagtggtcagggtatagcgggagttttgttcagcatccagaactaacagggctggattgtataagaccttgcttctgggttgaactaataagagggcgacccgaagagaacacaatctggactagcgggagcagcatatccttttgtggtgtaaacagtgacactgtgggttggtcttggccagacggtgctgagttgccatttaccattgacaagtaa

SEQ ID NO: 56 - protein, 512 aa, synthetic construct
  source 1..512: note = N1-MPP (with his tag)
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLGGGSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 57 - DNA, 1521 bp, synthetic construct
  source 1..1521: mol_type = other DNA; note = N1-MPP (no his tag)
atgctgctcgtcaaccaatcccaccagggcttcaacaaggaacacacttctaagatggtctccgctatcgtgctctacgtgctgctcgctgccgctgcccactcagctttcgctgccgacccaggcgatcactacgacgacgaactgttctccgacgtgcaggacatcaagaccgctctggctaagatccacgaggacaaccagaagatcatctccaagctggaatccctgctgctgctgaagggcgaagtcgagtccatcaagaagcagatcaaccgccagaacatctccatctccaccttggagggtcacctgtcctccatcatgatcgctatccctggcctgggcggcggctccgtgaaattagcgggcaattcctctctctgccctgttagtggatgggctatatacagtaaagacaacagtgtaagaatcggttccaagggggatgtgtttgtcataagggaaccattcatatcatgctctcccttggaatgcagaaccttcttcttgactcaaggggccttgctaaatgacaaacattccaatggaaccattaaagacaggagcccataccgaaccctaatgagctgtcctattggtgaagttccctctccatacaactcaagatttgagtcagtcgcttggtcagcaagtgcttgtcatgatggcatcaattggctaacaattggaatttctggcccagacagtggggcagtggctgtgttaaagtacaatggcataataacagacactatcaagagttggaggaacaatatattgagaacacaagagtctgaatgtgcatgtgtaaatggttcttgctttaccataatgaccgatggaccaagtgatggacaggcctcatacaaaatcttcagaatagaaaagggaaagataatcaaatcagtcgaaatgaaagcccctaattatcactatgaggaatgctcctgttaccctgattctagtgaaatcacatgtgtgtgcagggataactggcatggctcgaatcgaccgtgggtgtctttcaaccagaatctggaatatcagatgggatacatatgcagtggggttttcggagacaatccacgccctaatgataagacaggcagttgtggtccagtatcgtctaatggagcaaatggagtaaaaggattttcattcaaatacggcaatggtgtttggatagggagaactaaaagcattagttcaagaaaaggttttgagatgatttgggatccgaatggatggactgggactgacaataaattctcaataaagcaagatatcgtaggaataaatgagtggtcagggtatagcgggagttttgttcagcatccagaactaacagggctggattgtataagaccttgcttctgggttgaactaataagagggcgacccgaagagaacacaatctggactagcgggagcagcatatccttttgtggtgtaaacagtgacactgtgggttggtcttggccagacggtgctgagttgccatttaccattgacaagtaa

SEQ ID NO: 58 - protein, 506 aa, synthetic construct
  source 1..506: note = N1-MPP (no his tag)
MLLVNQSHQGFNKEHTSKMVSAIVLYVLLAAAAHSAFAADPGDHYDDELFSDVQDIKTALAKIHEDNQKIISKLESLLLLKGEVESIKKQINRQNISISTLEGHLSSIMIAIPGLGGGSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 59 - DNA, 1575 bp, synthetic construct
  source 1..1575: mol_type = other DNA; note = N2-MPP
atgctgctcgtcaaccaatcccaccagggcttcaacaaggaacacacttctaagatggtctccgctatcgtgctctacgtgctgctcgctgccgctgcccactcagctttcgctgccgacccacaccaccaccaccaccacggcgatcactacgacgacgaactgttctccgacgtgcaggacatcaagaccgctctggctaagatccacgaggacaaccagaagatcatctccaagctggaatccctgctgctgctgaagggcgaagtcgagtccatcaagaagcagatcaaccgccagaacatctccatctccaccttggagggtcacctgtcctccatcatgatcgctatccctggcctgtctctcgtgcccaggggatcaccttctagaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacaataaacaacgtgcattcaaataacacagcacgtgataggacccctcatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagtttcatttacaatgggaggcttgtagatagtgttgtttcatggtccaaagatattctcaggacccaggagtcagaatgcgtttgtatcaatggaacttgtacagtagtaatgactgatggaaatgctacaggaaaagctgatactaaaatattattcattgaggaggggaaaatcgttcatactagcaaattgtcaggaagtgctcagcatgtcgaagagtgctcttgctatcctcgataccctggtgtcagatgtgtctgcagagacaactggaaaggatccaaccggcccatcgtagatataaacataaaggatcatagcattgtttccagttatgtgtgttcaggacttgttggagacacacccagaaaaaccgacagctccagcagcagccattgcttgaatcctaacaatgaaaaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataa

SEQ ID NO: 60 - protein, 524 aa, synthetic construct
  source 1..524: note = N2-MPP
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLSLVPRGSPSRICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 61 - DNA, 1578 bp, synthetic construct
  source 1..1578: mol_type = other DNA; note = B-MPP
atgctgctcgtcaaccaatcccaccagggcttcaacaaggaacacacttctaagatggtctccgctatcgtgctctacgtgctgctcgctgccgctgcccactcagctttcgctgccgacccacaccaccaccaccaccacggcgatcactacgacgacgaactgttctccgacgtgcaggacatcaagaccgctctggctaagatccacgaggacaaccagaagatcatctccaagctggaatccctgctgctgctgaagggcgaagtcgagtccatcaagaagcagatcaaccgccagaacatctccatctccaccttggagggtcacctgtcctccatcatgatcgctatccctggcctgtctctcgtgcccaggggatcaccttctagacttcttctcccagaaccggagtggacatacccgcgtttatcttgcccgggctcaacctttcagaaagcactcctaattagccctcatagattcggagaaaccaaaggaaactcagctcccttgataataagggaaccttttgttgcttgtggaccaaatgaatgcaaacactttgctttaacccattatgcagcccaaccagggggatactacaatggaacaagaggagacagaaacaagctgaggcatctaatttcagtcaaattgggcaaaatcccaacagtagagaactccattttccacatggcagcatggagcgggtccgcgtgccatgatggtaaggaatggacatatatcggagttgatggccctgacaataatgcattgctcaaagtaaaatatggagaagcatatactgacacataccattcctatgcaaacaacatcctaagaacacaagaaagtgcctgcaattgcatcgggggaaattgttatctaatgataactgatggctcagcttcaggtgttagtgaatgcagatttcttaagattcgagagggccgaataataaaagaaatatttccaacaggaagagtaaaacacactgaggaatgcacatgcggatttgccagcaataaaaccatagaatgtgcctgtagagacaacaggtacacagcaaaaagaccttttgtcaaattaaacgtggagactgatacagcagaaataaggttgatgtgcacagatacctatttggacacccccagaccaaatgatggaagcataacaggcccttgtgaatctgatggggacaaagggagtggaggcatcaagggaggatttgttcatcaaagaatgaaatccaagattggaaggtggtactctcgaacgatgtctcaaactgaaaggatggggatgggactgtatgtcaagtatggtggagacccatgggctgacagtgatgccctagcttttagtggagtaatggtttcaatgaaagaacctggttggtattcctttggcttcgaaataaaagataagaaatgcgatgtcccctgtattgggatagagatggtacatgatggtggaaaagagacttggcactcagcagcaacagccatttactgtttaatgggctcaggacagctgctgtgggacactgtcacaggtgttgacatggctctgtaa

SEQ ID NO: 62 - protein, 525 aa, synthetic construct
  source 1..525: note = B-MPP
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLSLVPRGSPSRLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 63 - protein, 10 aa, synthetic construct
  source 1..10: note = Cleavage site
SLVPRGSPSR

SEQ ID NO: 64 - protein, 393 aa, synthetic construct
  source 1..393: note = Globular head domain of NA of influenza virus A/Kansas/14/2017
ICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 65 - protein, 394 aa, synthetic construct
  source 1..394: note = Globular head domain of NA of influenza virus B/Colorado/06/2017
LLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 66 - protein, 388 aa, synthetic construct
  source 1..388: note = Globular head domain of NA of influenza virus A/Michigan/45/15
SVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

(19) United States
(12) Patent Application Publication
(10) Pub. No.: US 2026/0275315 A1
(43) Pub. Date: Sep. 17, 2026

(54) RECOMBINANT NEURAMINIDASE AND USES THEREOF

(71) Applicant: ICAHN SCHOOL OF MEDICINE AT MOUNT SINAI, New York, NY (US)

(72) Inventors: Peter PALESE, New York, NY (US); Adolfo GARCIA-SASTRE, New York, NY (US); Florian KRAMMER, New York, NY (US)

(73) Assignee: ICAHN SCHOOL OF MEDICINE AT MOUNT SINAI, New York, NY (US)

(21) Appl. No.: 19/679,512

(22) Filed: May 15, 2026

Related U.S. Application Data
(62) Division of application No. 17/770,591, filed on Oct. 21, 2020, now Pat. No. 12,655,408.
(60) Provisional application No. 62/924,511, filed on Oct. 22, 2019.

(51) Int. Cl. C12N 9/24; C07K 14/005

(52) U.S. Cl. CPC C12N 9/2402; C07K 14/005; C12Y 302/01018; C07K 2319/02; C07K 2319/03; C07K 2319/21; C07K 2319/43; C12N 2760/16122

(57) ABSTRACT

In one aspect, provided herein are recombinant neuraminidases comprising an ectodomain of influenza virus neuraminidase with amino acid substitutions or insertions of cysteines in the stalk domain to generate a more stable, tetrameric influenza virus neuraminidase. In specific embodiments, the influenza virus neuraminidase further comprises influenza virus neuraminidase transmembrane and cytoplasmic domains. In another aspect, provided herein are recombinant neuraminidase comprising a globular head domain of influenza virus neuraminidase and a tetramerization domain, wherein the recombinant neuraminidase lacks influenza virus neuraminidase stalk, transmembrane and cytoplasmic domains. In another aspect, provided herein are methods of immunizing against influenza virus using such recombinant neuraminidases or compositions thereof.

### CROSS-REFERENCE TO RELATED APPLICATIONS

This application claims the benefit of U.S. Provisional Patent Application No. 62/924,511, filed Oct. 22, 2019, which is incorporated by reference herein in its entirety.

### STATEMENT REGARDING FEDERALLY SPONSORED RESEARCH

This invention was made with government support under award HHSN272201400008C, AI097092-07, R01AI145870-01 and 75N93019C00051 awarded by the National Institutes of Health. The government has certain rights in this invention.

### SEQUENCE LISTING

This application incorporates by reference in its entirety a Sequence Listing submitted with this application as a text filed entitled “06923-305-228_SEQ LISTING.txt”, created on Oct. 21, 2020, and is 226,341 bytes bytes in size.

### 1. INTRODUCTION

In one aspect, provided herein are recombinant neuraminidases comprising an ectodomain of influenza virus neuraminidase with amino acid substitutions or insertions of cysteines in the stalk domain to generate a more stable, tetrameric influenza virus neuraminidase. In specific embodiments, the influenza virus neuraminidase further comprises influenza virus neuraminidase transmembrane and cytoplasmic domains. In another aspect, provided herein are recombinant neuraminidase comprising a globular head domain of influenza virus neuraminidase and a tetramerization domain, wherein the recombinant neuraminidase lacks influenza virus neuraminidase stalk, transmembrane and cytoplasmic domains. In another aspect, provided herein are methods of immunizing against influenza virus using such recombinant neuraminidases or compositions thereof.

### 2. BACKGROUND

Influenza viruses are enveloped RNA viruses that belong to the family of Orthomyxoviridae (Palese and Shaw (2007) Orthomyxoviridae: The Viruses and Their Replication, 5th ed. Fields' Virology, edited by B. N. Fields, D. M. Knipe and P. M. Howley. Wolters Kluwer Health/Lippincott Williams & Wilkins, Philadelphia, USA, p1647-1689). The natural host of influenza A viruses are mainly avians, but influenza A viruses (including those of avian origin) also can infect and cause illness in humans and other animal hosts (bats, canines, pigs, horses, sea mammals, and mustelids). For example, the H5N1 avian influenza A virus circulating in Asia has been found in pigs in China and Indonesia and has also expanded its host range to include cats, leopards, and tigers, which generally have not been considered susceptible to influenza A (CIDRAP—Avian Influenza: Agricultural and Wildlife Considerations). The occurrence of influenza virus infections in animals could potentially give rise to human pandemic influenza strains.

Influenza A and B viruses are major human pathogens, causing a respiratory disease that ranges in severity from sub-clinical infection to primary viral pneumonia which can result in death. The clinical effects of infection vary with the virulence of the influenza strain and the exposure, history, age, and immune status of the host. The cumulative morbidity and mortality caused by seasonal influenza is substantial due to the relatively high attack rate. In a normal season, influenza can cause between 3-5 million cases of severe illness and up to 500,000 deaths worldwide (World Health Organization (2003) Influenza: Overview; March 2003). In the United States, influenza viruses infect an estimated 10-15% of the population (Glezen and Couch RB (1978) Interpandemic influenza in the Houston area, 1974-76. N Engl J Med 298: 587-592; Fox et al. (1982) Influenza virus infections in Seattle families, 1975-1979. II. Pattern of infection in invaded households and relation of age and prior antibody to occurrence of infection and related illness. Am J Epidemiol 116: 228-242) and are associated with approximately 30,000 deaths each year (Thompson W W et al. (2003) Mortality Associated with Influenza and Respiratory Syncytial Virus in the United States. JAMA 289: 179-186; Belshe (2007) Translational research on vaccines: influenza as an example. Clin Pharmacol Ther 82: 745-749).

In addition to annual epidemics, influenza viruses are the cause of infrequent pandemics. For example, influenza A viruses can cause pandemics such as those that occurred in 1918, 1957, 1968, and 2009. Due to the lack of pre-formed immunity against the major viral antigen, hemagglutinin (HA), pandemic influenza can affect greater than 50% of the population in a single year and often causes more severe disease than epidemic influenza. A stark example is the pandemic of 1918, in which an estimated 50-100 million people were killed (Johnson and Mueller (2002) Updating the Accounts: Global Mortality of the 1918-1920 “Spanish” Influenza Pandemic Bulletin of the History of Medicine 76: 105-115). Since the emergence of the highly pathogenic avian H5N1 influenza virus in the late 1990s (Claas et al. (1998) Human influenza A H5N1 virus related to a highly pathogenic avian influenza virus. Lancet 351: 472-7), there have been concerns that it may be the next pandemic virus.

Seasonal vaccination is currently the most effective intervention against influenza (Gross et al., Ann Intern Med, 1995, 123(7): p. 518-27; Ogburn et al., J Reprod Med, 2007, 52(9): p. 753-6; Jefferson et al., Lancet, 2005. 366(9492): p. 1165-74; Beyer et al., Vaccine, 2013, 31(50): p. 6030-3; Nichol et al., N Engl J Med, 1995. 333(14): p. 889-93; Jefferson et al., Lancet, 2005. 365(9461): p. 773-80), yet overall vaccine effectiveness was only 36% in the recent 2017-2018 season (Flannery et al., MMWR Morb Mortal Wkly Rep, 2018. 67(6): p. 180-185). However, current vaccination approaches rely on achieving a good match between circulating strains and the isolates included in the vaccine. Such a match is often difficult to attain due to a combination of factors. First, influenza viruses are constantly undergoing change: every 3-5 years the predominant strain of influenza A virus is replaced by a variant that has undergone sufficient antigenic drift to evade existing antibody responses. Isolates to be included in vaccine preparations must therefore be selected each year based on the intensive surveillance efforts of the World Health Organization (WHO) collaborating centers. Second, to allow sufficient time for vaccine manufacture and distribution, strains must be selected approximately six months prior to the initiation of the influenza season. Often, the predictions of the vaccine strain selection committee are inaccurate, resulting in a substantial drop in the efficacy of vaccination.

The neuraminidase (NA) of influenza viruses is a homo-tetrameric, type II transmembrane protein (Fields S, Winter G, Brownlee G G. 1981. Structure of the neuraminidase gene in human influenza virus A/PR/8/34. Nature 290:213-7; Ward C W, Elleman T C, Azad A A. 1982. Amino acid sequence of the Pronase-released heads of neuraminidase subtype N2 from the Asian strain A/Tokyo/3/67 of influenza virus. Biochem J 207:91-5). The tetrameric form of the NA has the enzymatic function of removing sialic acids from mucus, which is crucial to prevent immobilization of the virus in the respiratory tract, and to release virus progenies from infected cells (Palese P, Tobita K, Ueda M, Compans R W. 1974. Characterization of temperature sensitive influenza virus mutants defective in neuraminidase. Virology 61:397-410). Four identical monomeric polypeptides each approximately 470 amino acids assemble to form tetrameric NA (McAuley et al., 2019, Frontiers in Microbiology 10: 39). The four monomers each have four distinct structural domains: the cytoplasmic tail, the transmembrane region, the stalk, and the catalytic head (id.). The ectodomain of NA is composed of the globular head domain and stalk domain of NA.

Antiviral drugs that target the enzymatic function of NA have been shown to reduce virus load (Moscona A. 2005. Neuraminidase inhibitors for influenza. N Engl J Med 353:1363-73; Gubareva L V, Kaiser L, Hayden F G. 2000. Influenza virus neuraminidase inhibitors. Lancet 355:827-35). The influenza virus NA is also an excellent immune target as the NA harbors conserved epitopes (Chen Y Q, Wohlbold T J, Zheng N Y, Huang M, Huang Y, Neu K E, Lee J, Wan H, Rojas K T, Kirkpatrick E, Henry C, Palm A E, Stamper C T, Lan L Y, Topham D J, Treanor J, Wrammert J, Ahmed R, Eichelberger M C, Georgiou G, Krammer F, Wilson P C. 2018. Influenza Infection in Humans Induces Broadly Cross-Reactive and Protective Neuraminidase-Reactive Antibodies. Cell 173:417-429.e10; Wohlbold T J, Podolsky K A, Chromikova V, Kirkpatrick E, Falconieri V, Meade P, Amanat F, Tan J, tenOever BR, Tan G S, Subramaniam S, Palese P, Krammer F. 2017. Broadly protective murine monoclonal antibodies against influenza B virus target highly conserved neuraminidase epitopes. Nat Microbiol 2:1415-1424). Therefore, antibodies that inhibit neuraminidase activity and bind to the protein would likely be protective. Indeed, several studies have shown that NA-specific antibodies that are neuraminidase inhibitory (NAI) correlate with protection against influenza viruses in both animals and humans (Jacobsen H, Rajendran M, Choi A, Sjursen H, Brokstad K A, Cox R J, Palese P, Krammer F, Nachbagauer R. 2017. Influenza Virus Hemagglutinin Stalk-Specific Antibodies in Human Serum are a Surrogate Marker for In Vivo Protection in a Serum Transfer Mouse Challenge Model. MBio 8; Monto A S, Petrie J G, Cross R T, Johnson E, Liu M, Zhong W, Levine M, Katz J M, Ohmit S E. 2015. Antibody to Influenza Virus Neuraminidase: An Independent Correlate of Protection. J Infect Dis 212:1191-9; Kilbourne E D, Johansson B E, Grajower B. 1990. Independent and disparate evolution in nature of influenza A virus hemagglutinin and neuraminidase glycoproteins. Proc Natl Acad Sci USA 87:786-90; Couch RB, Atmar R L, Franco L M, Quarles J M, Wells J, Arden N, Nino D, Belmont J W. 2013. Antibody correlates and predictors of immunity to naturally occurring influenza in humans and the importance of antibody to the neuraminidase. J Infect Dis 207:974-81; Walz L, Kays S K, Zimmer G, von Messling V. 2018. Neuraminidase-Inhibiting Antibody Titers Correlate with Protection from Heterologous Influenza Virus Strains of the Same Neuraminidase Subtype. J Virol 92). Antibodies against NA have been shown to reduce viral shedding in infected individuals (Maier H E, Nachbagauer R, Kuan G, Ng S, Lopez R, Sanchez N, Stadlbauer D, Gresh L, Schiller A, Rajabhathor A, Ojeda S, Guglia A F, Amanat F, Balmaseda A, Krammer F, Gordon A. 2019. Pre-existing anti-neuraminidase antibodies are associated with shortened duration of influenza A (H1N1)pdm virus shedding and illness in naturally infected adults. Clin Infect Dis.) and guinea pigs (McMahon M, Kirkpatrick E, Stadlbauer D, Strohmeier S, Bouvier N M, Krammer F. 2019. Mucosal Immunity against Neuraminidase Prevents Influenza B Virus Transmission in Guinea Pigs. MBio 10). However, current inactivated influenza virus vaccines (IIVs) induce limited NA-specific antibody responses, while most of the immune response is against the HA. This is likely due to the lower immunogenicity of the NA compared to that of HA in the context of IIVs and the lack of standardization of the NA content during vaccine production (Wohlbold T J, Nachbagauer R, Xu H, Tan G S, Hirsh A, Brokstad K A, Cox R J, Palese P, Krammer F. 2015. Vaccination with Adjuvanted Recombinant Neuraminidase Induces Broad Heterologous, but Not Heterosubtypic, Cross-Protection against Influenza Virus Infection in Mice. MBio 6; Krammer F, Fouchier RAM, Eichelberger M C, Webby R J, Shaw-Saliba K, Wan H, Wilson P C, Compans R W, Skountzou I, Monto A S. 2018. NAction! How Can Neuraminidase-Based Immunity Contribute to Better Influenza Virus Vaccines? MBio 9). It was not clear whether the in-correctly folded monomeric NA could elicit protective antibody responses. But it is speculated that a correctly folded tetrameric NA is required, as anti-N1 or N2 titers have been observed to correlate with enzymatically active forms of the NAs in the vaccines (Krammer F, Fouchier RAM, Eichelberger M C, Webby R J, Shaw-Saliba K, Wan H, Wilson P C, Compans R W, Skountzou I, Monto A S. 2018. NAction!How Can Neuraminidase-Based Immunity Contribute to Better Influenza Virus Vaccines?MBio 9; Marcelin G, Sandbulte M R, Webby R J. 2012. Contribution of antibody production against neuraminidase to the protection afforded by influenza vaccines. Rev Med Virol 22:267-79).

Thus, there is a need for therapies to prevent and treat influenza virus infections and influenza virus diseases.

### 3. SUMMARY

In one aspect, provided herein is are recombinant neuraminidases comprising a mutated influenza virus neuraminidase ectodomain, wherein the mutated ectodomain comprises an influenza virus neuraminidase ectodomain comprising one, two or more amino acid substitutions to cysteine at one, two or more amino acid residues found in the stalk domain of the ectodomain. In one embodiment, provided herein is a recombinant neuraminidase comprising a mutated influenza virus neuraminidase ectodomain, wherein the mutated ectodomain comprises an influenza virus neuraminidase ectodomain comprising an amino acid substitution to cysteine at amino acid residue 48 or 50 of an N1 subtype or at an amino acid residue corresponding to amino acid residue 48 or 50 of influenza virus A/Puerto Rico/08/1934. In another embodiment, provided herein is a recombinant neuraminidase comprising a mutated influenza virus neuraminidase ectodomain, wherein the mutated ectodomain comprises an influenza virus neuraminidase ectodomain comprising amino acid substitutions to cysteine at amino acid residues 48 and 50 of an N1 subtype or at amino acid residues corresponding to amino acid residues 48 and 50 of influenza virus A/Puerto Rico/08/1934. In certain embodiments, the influenza virus neuraminidase ectodomain is of subtype N1 or N2. In other embodiments, the influenza virus neuraminidase ectodomain is of subtype N3, N4, N5, N6, N7, N8 or N9 subtype. In some embodiments, the recombinant neuraminidase further comprises the influenza virus neuraminidase transmembrane and cytoplasmic domains. In other embodiments, the recombinant neuraminidase further comprises a tetramerization domain. In a specific embodiment, the tetramerization domain comprises a measles virus phosphoprotein tetramerization domain or a Sendai virus phosphoprotein tetramerization domain.

In another embodiment, provided herein is a recombinant neuraminidase comprising a mutated influenza virus neuraminidase ectodomain, wherein the mutated ectodomain comprises an influenza virus neuraminidase ectodomain comprising an amino acid substitution to cysteine at amino acid residue 61 of an N1 subtype or at an amino acid residue corresponding to amino acid residue 61 of influenza virus A/Puerto Rico/08/1934. In some embodiments, the mutated influenza virus neuraminidase ectodomain further comprises an amino acid substitution to a cysteine at amino acid residue 48 of an N1 subtype or at an amino acid residue corresponding to amino acid residue 48 of influenza virus A/Puerto Rico/08/1934. In certain embodiments, the influenza virus neuraminidase ectodomain is of subtype N1 or N2. In other embodiments, the influenza virus neuraminidase ectodomain is of subtype N3, N4, N5, N6, N7, N8 or N9 subtype. In some embodiments, the recombinant neuraminidase further comprises the influenza virus neuraminidase transmembrane and cytoplasmic domains. In other embodiments, the recombinant neuraminidase further comprises a tetramerization domain. In a specific embodiment, the tetramerization domain comprises a measles virus phosphoprotein tetramerization domain or a Sendai virus phosphoprotein tetramerization domain.

In another embodiment, provided herein is a recombinant neuraminidase comprising a mutated influenza virus neuraminidase ectodomain and a tetramerization domain, wherein the mutated ectodomain comprises an influenza virus neuraminidase ectodomain comprising an amino acid substitution to a cysteine at amino acid residue 48 of an N1 subtype or at an amino acid residue corresponding to amino acid residue 48 of influenza virus A/Puerto Rico/08/1934. In certain embodiments, the tetramerization domain comprises a measles virus phosphoprotein tetramerization domain or a Sendai virus phosphoprotein tetramerization domain. In certain embodiments, the influenza virus neuraminidase ectodomain is of subtype N1 or N2. In other embodiments, the influenza virus neuraminidase ectodomain is of subtype N3, N4, N5, N6, N7, N8 or N9 subtype.

In another embodiment, provided herein is a recombinant neuraminidase comprising a mutated influenza virus neuraminidase ectodomain, wherein the mutated ectodomain comprises an influenza virus neuraminidase ectodomain comprising an amino acid substitution to cysteine at amino acid residue 52 of an N2 subtype or at an amino acid residue corresponding to amino acid residue 52 of influenza virus A/Hong Kong/4801/2014, wherein the ectodomain is from an influenza A virus N2, N3, N4, N5, N6, N7, N8 or N9 subtype. In certain embodiments, the mutated ectodomain further comprises an amino acid substitution to cysteine at amino acid residue 54 of an N2 subtype or at amino acid residue corresponding to amino acid residue 54 of A/Hong Kong/4801/2014. In another embodiment, provided herein is a recombinant neuraminidase comprising a mutated influenza virus neuraminidase ectodomain, wherein the mutated ectodomain comprises an influenza virus neuraminidase ectodomain comprising an amino acid substitution to cysteine at amino acid residue 54 of an N2 subtype or at an amino acid residue corresponding to amino acid residue 54 of A/Hong Kong/4801/2014, wherein the ectodomain is from an influenza A virus N2, N3, N4, N5, N6, N7, N8 or N9 subtype. In some embodiments, the recombinant neuraminidase further comprises the influenza virus neuraminidase transmembrane and cytoplasmic domains. In other embodiments, the recombinant neuraminidase further comprises a tetramerization domain. In a specific embodiment, the tetramerization domain comprises a measles virus phosphoprotein tetramerization domain or a Sendai virus phosphoprotein tetramerization domain.

In another aspect, provided herein is a recombinant neuraminidase comprising an influenza virus neuraminidase globular head domain and a tetramerization domain, wherein the recombinant neuraminidase lacks all or a portion of the influenza virus neuraminidase stalk (e.g., 25, 30, 35, 40, 45, 50, 55, 60 or more amino acid residues of the influenza neuraminidase stalk, or 25-50, 30-50, 40-50, 30-60, 40-60 or 50-60 amino acid residues of the influenza neuraminidase stalk), the transmembrane domain and the cytoplasmic domain. In one embodiment, the tetramerization domain comprises a paramyxovirus protein tetramerization domain. In specific embodiments, the paramyxovirus protein tetramerization domain is a tetramerization domain of a paramyxovirus phosphoprotein (e.g., a Nipah virus phosphoprotein, a Hendra virus phosphoprotein, a respiratory syncytial virus phosphoprotein, human parainfluenza virus (hPIV) phosphoprotein, bovine parainfluenza virus phosphoprotein, a mumps virus phosphoprotein, a Cedar virus phosphoprotein, a Ghana virus phosphoprotein, a Newcastle disease virus phosphoprotein, a canine distemper virus phosphoprotein, or a Peste des petits ruminants virus (PPRV) phosphoprotein). In another embodiment, provided herein is a recombinant neuraminidase comprising an influenza virus neuraminidase globular head domain and a tetramerization domain, wherein the recombinant neuraminidase lacks all or a portion of the influenza virus neuraminidase stalk (e.g., 25, 30, 35, 40, 45, 50, 55, 60 or more amino acid residues of the influenza neuraminidase stalk, or 25-50, 30-50, 40-50, 30-60, 40-60 or 50-60 amino acid residues of the influenza neuraminidase stalk), the transmembrane domain and the cytoplasmic domain, and wherein the tetramerization domain comprises a measles virus phosphoprotein tetramerization domain or a Sendai virus phosphoprotein tetramerization domain. In certain embodiments, the influenza virus neuraminidase ectodomain is of subtype N1 or N2. In other embodiments, the influenza virus neuraminidase ectodomain is of subtype N3, N4, N5, N6, N7, N8 or N9 subtype.

In another aspect, provided herein is a recombinant neuraminidase comprising an influenza virus neuraminidase globular head domain and a tetramerization domain, wherein the recombinant neuraminidase lacks influenza virus neuraminidase stalk, transmembrane and cytoplasmic domains. In one embodiment, the tetramerization domain comprises a paramyxovirus phosphoprotein tetramerization domain. In another embodiment, provided herein is a recombinant neuraminidase comprising an influenza virus neuraminidase globular head domain and a paramyxovirus protein tetramerization domain (e.g., a paramyxovirus protein tetramerization domain), wherein the recombinant neuraminidase lacks of influenza virus neuraminidase stalk domain, transmembrane domain and cytoplasmic domain. In specific embodiments, the paramyxovirus phosphoprotein is a Nipah virus phosphoprotein, a Hendra virus phosphoprotein, a respiratory syncytial virus phosphoprotein, human parainfluenza virus (hPIV) phosphoprotein, bovine parainfluenza virus phosphoprotein, a mumps virus phosphoprotein, a Cedar virus phosphoprotein, a Ghana virus phosphoprotein, a Newcastle disease virus phosphoprotein, a canine distemper virus phosphoprotein, or a Peste des petits ruminants virus (PPRV) phosphoprotein. In another embodiment, provided herein is a recombinant neuraminidase comprising an influenza virus neuraminidase globular head domain and a tetramerization domain, wherein the recombinant neuraminidase lacks of influenza virus neuraminidase stalk domain, transmembrane domain and cytoplasmic domain, and wherein the tetramerization domain comprises a measles virus phosphoprotein tetramerization domain or a Sendai virus phosphoprotein tetramerization domain. In certain embodiments, the influenza virus neuraminidase ectodomain is of subtype N1 or N2. In other embodiments, the influenza virus neuraminidase ectodomain is of subtype N3, N4, N5, N6, N7, N8 or N9 subtype.

In certain embodiments, a recombinant neuraminidase described herein comprises a signal peptide. In some embodiments, a recombinant neuraminidase described herein comprises a histidine tag, a Flag tag, or other purification tag. In certain embodiments, a recombinant neuraminidase described herein comprises a cleavage site, such as described in, e.g., Section 6, infra. In some embodiments, a recombinant neuraminidase described herein comprises a cleavage site, such as described in, e.g., Section 6, infra and a histidine tag, a Flag tag, or other purification tag. In specific embodiments, a recombinant neuraminidase described herein is isolated.

In certain embodiments, provided herein is a recombinant neuraminidase comprising the amino acid sequence of SEQ ID NO: 27, 56, 58, 60 or 62. In a specific embodiment, a recombinant neuraminidase comprises the amino acid sequence of SEQ ID NO: 27, 56 or 58.

In another aspect, provided herein is a nucleic acid sequence comprising a nucleotide sequence encoding a recombinant neuraminidase described herein. In a specific embodiment, the nucleic acid sequence is isolated.

In certain embodiments, provided herein is a nucleic acid sequence comprising the nucleotide sequence of SEQ ID NO: 55, 57, 59, or 61. In a specific embodiment, a nucleic acid sequence provided herein comprises the nucleic acid sequence comprises the nucleotide sequence of SEQ ID NO: 55 or 57.

In another aspect, provided herein is an expression vector comprising a nucleic acid sequence that comprises a nucleotide sequence encoding a recombinant neuraminidase described herein. In a specific embodiment, the expression vector facilitates expression of the recombinant neuraminidase in a suitable substrate.

In certain embodiments, provided herein is an expression vector comprising a nucleic acid sequence, wherein the nucleic acid sequence comprises the nucleotide sequence of SEQ ID NO: 55, 57, 59, or 61. In a specific embodiment, provided herein is an expression vector comprising a nucleic acid sequence, wherein the nucleic acid sequence comprises the nucleotide sequence of SEQ ID NO: 55 or 57.

In another aspect, provided herein are host cells capable of expressing a nucleic acid sequence comprising a nucleotide sequence encoding a recombinant neuraminidase described herein. In a specific embodiment, provided herein is a host cell expressing the nucleic acid sequence comprising a nucleotide sequence encoding a recombinant neuraminidase described herein.

In another aspect, provided herein is a recombinant influenza virus comprising a recombinant neuraminidase described herein. In one embodiment, provided herein is a recombinant influenza virus comprising a genome, wherein the genome comprises a gene segment comprising a nucleic acid sequence encoding the recombinant neuraminidase described herein such that the recombinant neuraminidase is expressed by a cell infected with the recombinant influenza virus. In another embodiment, provided herein is a recombinant influenza virus comprising a neuraminidase, wherein the neuraminidase comprises a mutated influenza virus neuraminidase ectodomain, wherein the mutated ectodomain comprises an influenza virus neuraminidase ectodomain comprising an amino acid substitution to cysteine at amino acid residue 48 of an N1 subtype or at an amino acid residue corresponding to amino acid residue 48 of influenza virus A/Puerto Rico/08/1934. In another aspect, provided herein is a recombinant influenza virus comprising a genome, wherein the genome comprises a gene segment comprising a nucleotide sequence encoding a neuraminidase such that the neuraminidase is expressed by an infected cell, wherein the neuraminidase comprises a mutated influenza virus neuraminidase ectodomain, wherein the mutated ectodomain comprises an influenza virus neuraminidase ectodomain comprising an amino acid substitution to cysteine at amino acid residue 48 of an N1 subtype or at amino acid residue corresponding to amino acid residue 48 of influenza virus A/Puerto Rico/08/1934. In certain embodiments, a recombinant influenza virus described herein is inactivated. In specific embodiments, a recombinant influenza virus described herein is split. In other embodiments, a recombinant influenza virus described herein is a live attenuated influenza virus. In some embodiments, a recombinant influenza virus described herein is a recombinant influenza A virus. In a specific embodiment, a recombinant influenza virus described herein is an H1 or H3 subtype. In other embodiments, a recombinant influenza virus described herein is an influenza B virus.

In another aspect, provided herein is an immunogenic composition comprising the neuraminidase described herein. In some embodiments, the immunogenic composition further comprises a trivalent inactivated influenza vaccine (TIV), quadrivalent inactivated influenza virus vaccine (QIV), or recombinant influenza virus vaccine. In some embodiments, the immunogenic composition further comprises an adjuvant.

In another aspect, provided herein is an immunogenic composition comprising a nucleic acid sequence that comprises a nucleotide sequence encoding a recombinant neuraminidase described herein. In some embodiments, the immunogenic composition further comprises an adjuvant.

In another aspect, provided herein is an immunogenic composition comprising the recombinant influenza virus described herein. In some embodiments, the immunogenic composition further comprises an adjuvant.

In another aspect, provided herein is a method of immunizing against influenza virus, comprising administering a subject a dose of the immunogenic composition described herein. In another aspect, provided herein is a method of inducing an immune response against influenza virus, comprising administering a subject a dose of the immunogenic composition described herein. In some embodiments, the subject is administered a further dose of the immunogenic composition as a boost. In a specific embodiment, the subject is human. In another specific embodiment, the immunogenic composition is administered intramuscularly to the subject.

### 3.1 Terminology

As used herein, the term “nucleic acid” is intended to include DNA molecules (e.g., cDNA or genomic DNA) and RNA molecules (e.g., mRNA) and analogs of the DNA or RNA generated using nucleotide analogs. The nucleic acid can be single-stranded or double-stranded. In a specific embodiment, the nucleic acid may be self-replicating RNA.

As used herein, the terms “purified” and “isolated” when used in the context of a polypeptide (including an antibody) that is obtained from a natural source, e.g., cells, refers to a polypeptide which is substantially free of contaminating materials from the natural source, e.g., minerals, chemicals from the environment, and/or cellular materials from the natural source, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a polypeptide (including an antibody) that is chemically synthesized refers to a polypeptide which is substantially free of chemical precursors or other chemicals which are involved in the syntheses of the polypeptide. In a specific embodiment, a recombinant influenza virus NA is chemically synthesized. In another specific embodiment, a recombinant influenza virus NA is isolated.

As used herein, terms “subject” or “patient” are used interchangeably to refer to an animal (e.g., birds, reptiles, and mammals). In a specific embodiment, a subject is a bird. In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human. In another embodiment, a subject is a human infant. In another embodiment, a subject is a human child. In another embodiment, a subject is a human adult. In another embodiment, a subject is an elderly human. In another embodiment, a subject is a premature human infant.

As used herein, the term “premature human infant” refers to a human infant born at less than 37 weeks of gestational age.

As used herein, the term “seasonal influenza virus strain” refers to a strain of influenza virus to which a subject population is exposed to on a seasonal basis. In specific embodiments, the term seasonal influenza virus strain refers to a strain of influenza A virus. In specific embodiments, the term seasonal influenza virus strain refers to a strain of influenza virus that belongs to the H1 or the H3 subtype, i.e., the two subtypes that presently persist in the human subject population. In other embodiments, the term seasonal influenza virus strain refers to a strain of influenza B virus.

The terms “tertiary structure” and “quaternary structure” have the meanings understood by those of skill in the art. Tertiary structure refers to the three-dimensional structure of a single polypeptide chain. Quaternary structure refers to the three dimensional structure of a polypeptide having multiple polypeptide chains.

As used herein, in some embodiments, the phrase “wild-type” in the context of a viral polypeptide refers to a viral polypeptide that is found in nature and is associated with a naturally occurring virus.

As used herein, in some embodiments, the phrase “wild-type” in the context of a virus refers to the types of a virus that are prevalent, circulating naturally and producing typical outbreaks of disease. In other embodiments, the term “wild-type” in the context of a virus refers to a parental virus.

### 4. DESCRIPTION OF THE FIGURES

FIGS. 1A-1C. Reducing (FIG. 1A), non-reducing (FIG. 1B) and cross-linked (FIG. 1C) SDS-PAGE of measles virus phosphoprotein (MPP-N1) or the Sendai virus phosphoprotein (SPP-N1) tetramerization domain fused to N1 as well as a SEPPALLATA-like MADS domain transcription factor from Arabidopsis tetramerization domain fused to N1 (SMDTF-N1). VASP-N1 serves as positive control. MPP-N1, SPP-N1 and VASP-N1 are maintained as tetramers when cross-linked.

FIG. 2. NA activity of constructs with the measles virus phosphoprotein (MPP-N1) or the Sendai virus phosphoprotein (SPP-N1) tetramerization domain fused to N1. VASP-N1 serves as positive control, and irrelevant

FIG. 3. Human NI active antibodies, including pan-NA mAb 1G01, recognize MPP-1 in an ELISA on Ni-NTA plates.

FIG. 4. Protective effect of the measles virus phosphoprotein (MPP-N1) or the Sendai virus phosphoprotein (SPP-N1) tetramerization domain fused to N1 as vaccine in the mouse model. VASP-N1 serves as positive control, and irrelevant proteins served as negative control. % in parentheses indicates survival.

FIG. 5. NA activity of different NA-stalk cysteine mutants in an NA-Star assay. Area under the curve is shown as quantification of the activity.

FIGS. 6A-6B. NA contents of inactivated purified PR8 H1N1 viruses. (FIG. 6A) NA contents of inactivated purified viruses expressing WT, I48C, N50C, I48C_N50C, W61insertC or V62insertC NA were examined by western blot. (FIG. 6B) A combination of I48C with W61insertC results in more tetrameric and less trimeric contents of NA than other mutants. NA contents of inactivated purified viruses expressing WT, I48C, I48C_N50C, I48C_W61insert C NA were examined by western blot. The viral proteins in A and B were resolved on 10% SDS-PAGE. NR: non-reducing; R: reducing

FIG. 7. Amino acid sequence alignment of PR8 N1 and HK14 N2 (N1, aa 1-102 (SEQ ID NO:53); N2, aa 1-117 (SEQ ID NO:54)) to identify corresponding residues for cysteine substitutions in HK14 N2. The natural cysteines in N1 (C49) and N2 (C53) are boxed.

FIGS. 8A-8B. NA contents and NA activity of inactivated purified HK14 H3N2 viruses. (FIG. 8A) NA contents of WT, L52C and L52C_E54C viruses were examined by western blot. The viral proteins were resolved on 8%-16% SDS-PAGE. (FIG. 8B) NA activity of virus preparations. A NA-fluor neuraminidase activity assay was used to measure potential NA activity from 5 ng of each virus.

FIGS. 9A-9D. Alignment of the neuraminidases of the following influenza viruses A/PR/8/34 (N1 PR8; SEQ ID NO:29), A/Michigan/45/15 (N1_Mich15; SEQ ID NO:30), A/WSN/33 (N1_WSN; SEQ ID NO:31), A/Hong Kong/4801/14 (N2_HK14; SEQ ID NO:32), A/chicken/Bangladesh/19870/13 (N2_ck; SEQ ID NO:33), A/swine/Missouri/4296424/06A (N3_sw; SEQ ID NO:34), A/blue-winged teal/Guatemala/CIP049H113-74/13 (N3_bwt; SEQ ID NO:35), A/duck/Jiangxi/22676/13 (N3_duck; SEQ ID NO:36). A/duck/Hokkaido/222/14 (N4_duck; SEQ ID NO:37), A/ruddy turnstone/Delaware/AI03-378/03 (N4_rt; SEQ ID NO:38), A/American wigeon/California/HS007B/15 (N5_aw; SEQ ID NO:39), A/migratory duck/Jiangxi/6847/03 (N5_md; SEQ ID NO:40), A/Shenzhen/1/16 (N6_Sz; SEQ ID NO:41), A/Caspian seal/Russia/T1/12 (N6_cs; SEQ ID NO:42), A/chicken/Netherlands/1/03 (N7_cN; SEQ ID NO:43), A/chicken/Germany/R28/03 (N7_cG; SEQ ID NO:44), A/chicken/Netherlands/14015531/14 (N8_cN; SEQ ID NO:45), A/chicken/Laos/A0573/07 (N8_cL; SEQ ID NO:46), A/Anhui/2/13 (N9_AH13; SEQ ID NO:47), A/chicken/Dongguan/1143/14 (N9_ck; SEQ ID NO:48), and B/Malaysia/2506/04 (B_Mal; SEQ ID NO:49). C49 is shown in bold and underlined for N1_PR8.

FIG. 10. This figure depicts the immunization protocol administered to a female 6-8 week old BALB/c mice. In particular, recombinant N1-MPP (SEQ ID NO:27) was tested as a vaccine in a mouse model. The table in the figure lists the antigen administered for the various groups. Group 1 received recombinant N1-MPP at a 3 μg dose intramuscularly twice in a three week interval. Group 2 received the same antigen but mixed with the adjuvant AddaVax (see the depiction in the figure with the mice). Group 3 received recombinant N1-MPP admixed to a 1 μg dose (based on H1 HA) of a current seasonal quatrivalent influenza virus vaccine (QIV, brand: Flucelvax) twice. Group 4 received recombinant N1-MPP in one leg and the QIV in the second leg based on the assumption that mixing NA and QIV could render the NA immunosubdominant. Group 5 received QIV twice and group 6 received an irrelevant recombinant protein as negative control. Animals were bled after the prime and after the boost. Four weeks post boost, animals were challenged with 25× or 400×LD₅₀ of A/Singapore/GP1908/2015 IVR-180 (H1N1).

FIGS. 11A-11C. FIG. 11A depicts the results from an ELISA measuring the binding of sera from mice immunized in accordance with the protocol provided in the legend for FIG. 10 for groups 1 to 6 to recombinant N1-VASP, which comprises the globular head domain of influenza virus A/Michigan/45/15 NA and the human VASP tetramerization domain. FIG. 11B depicts the results from an ELISA measuring the binding of sera from mice immunized in accordance with the protocol provided in the legend for FIG. 10 for groups 1 to 6 to recombinant H1. FIG. 11C depicts the results from functional neuraminidase inhibition assays. In particular, the ability of sera from mice immunized in accordance with the protocol provided in the legend for FIG. 10 for groups 1 to 6 to inhibit the neuraminidase function of an H7N1 re-assortant virus that contains a matched N1 NA (influenza virus A/Michigan/45/15 NA), an H7 HA and PR8 sequences.

FIGS. 12A-12B. FIG. 12A depicts the weight loss of mice in groups 1 to 6 immunized as described in the legend for FIG. 10 and challenged as described in the legend for FIG. 10 with 25×LD₅₀ of A/Singapore/GP1908/2015 IVR-180 (H1N1). FIG. 12B depicts the survival of mice in groups 1 to 6 immunized as described in the legend for FIG. 10 and challenged as described in the legend for FIG. 10 with 25×LD₅₀ of A/Singapore/GP1908/2015 IVR-180 (H1N1).

FIGS. 13A-13B. FIG. 13A depicts the weight loss of mice in groups 2, 4, 5 and 6 immunized as described in the legend for FIG. 10 and challenged as described in the legend for FIG. 10 with 400×LD₅₀ of A/Singapore/GP1908/2015 IVR-180 (H1N1). FIG. 12B depicts the survival of mice in groups 2, 4, 5 and 6 immunized as described in the legend for FIG. 10 and challenged as described in the legend for FIG. 10 with 400×LD₅₀ of A/Singapore/GP1908/2015 IVR-180 (H1N1).

FIGS. 14A-14E. Recombinant N2-MPP (comprising the MPP tetramerization domain and globular head domain of influenza virus A/Kansas/14/2017 (H3N2) N2; SEQ ID NO:60) and recombinant B-MPP (comprising the MPP tetramerization domain and globular head domain of influenza virus B/Colorado/06/2017 NA; SEQ ID NO: 62). FIG. 14A shows the reactivity of broadly reactive anti-influenza B virus NA monoclonal antibodies to recombinant B-MPP. FIG. 14B shows the reactivity of broadly reactive anti-influenza A virus N2 NA monoclonal antibodies to recombinant N2-MPP. FIG. 14C shows the NA activity of recombinant N1-MPP (SEQ ID NO: 27), recombinant N2-MPP (SEQ ID NO: 60), and recombinant B-MPP (SEQ ID NO:62) in an NA-star assay. FIG. 14D shows recombinant N1-MPP (SEQ ID NO: 27), recombinant N2-MPP (SEQ ID NO:60), and recombinant B-MPP (SEQ ID NO:62) in a reducing SDS-PAGE. FIG. 14E shows recombinant N1-MPP (SEQ ID NO: 27), recombinant N2-MPP (SEQ ID NO:60), and recombinant B-MPP (SEQ ID NO:62) in a cross-linking SDS-PAGE.

FIG. 15. This figure provides the immunization regimen used to immunize mice with recombinant N1-MPP, recombinant N2-MPP, or recombinant B-MPP. With respect to the study of each of the three recombinant NA proteins (recombinant N1-MPP, recombinant N2-MPP, and recombinant B-MPP), three groups of mice were immunized. Mice in groups 1 and 2 were vaccinated with 3 μg of recombinant NA protein (recombinant N1-MPP, recombinant N2-MPP, or recombinant B-MPP, as indicated) intramuscularly twice in a three week interval. For one group, the recombinant NA was always given unadjuvanted, for the second group adjuvanted with AddaVax. A third group received irrelevant recombinant protein as a negative control. Three weeks after the boost the animals were challenged with 25×LD₅₀ of the specified challenge viruses.

FIGS. 16A-16D. FIG. 16A depicts the weight loss of mice vaccinated with N1-MPP as described in the legend for FIG. 15 and challenged as described in the legend for FIG. 15 with 25×LD₅₀ of A/Singapore/GP1908/2015 (H1N1). FIG. 16B depicts the survival of mice vaccinated with N1-MPP as described in the legend for FIG. 15 and challenged as described in the legend for FIG. 15 with 25×LD₅₀ of A/Singapore/GP1908/2015 (H1N1). FIG. 16C depicts the results from an ELISA measuring the binding of sera from mice immunized in accordance with the protocol provided in FIG. 15 and the legend for FIG. 15 for groups 1 to 3 to recombinant N1-VASP (which comprises the globular head domain of influenza virus A/Michigan/45/15 NA and the human VASP tetramerization domain). FIG. 16D depicts the results from functional neuraminidase inhibition assays. In particular, the ability of sera from mice vaccinated with N1-MPP as described in the legend for FIG. 15 to inhibit the neuraminidase function of an H7N1 re-assortant virus that contains a matched N1 NA was assessed.

FIG. 17A-17C. FIG. 17A depicts the weight loss of mice vaccinated with N2-MPP as described in the legend for FIG. 15 and challenged as described in the legend for FIG. 15 with 25×LD₅₀ of A/Switzerland/9715293/2013 (H3N2, mouse adapted). FIG. 17B depicts the survival of mice vaccinated with N2-MPP as described in the legend for FIG. 15 and challenged as described in the legend for FIG. 15 with 25×LD₅₀ of A/Switzerland/9715293/2013 (H3N2, mouse adapted). FIG. 17C depicts the results from an ELISA measuring the binding of sera from mice immunized in accordance with the protocol provided in FIG. 15 and the legend for FIG. 15 for groups 1 to 3 to recombinant N2-VASP (which comprises the globular head domain of influenza virus A/Kansas/14/2017 NA and the human VASP tetramerization domain).

FIGS. 18A-18C. FIG. 18A depicts the weight loss of mice vaccinated with B-MPP as described in the legend for FIG. 15 and challenged as described in the legend for FIG. 15 with 25×LD₅₀ of B/New York/PV01181/2018. FIG. 18B depicts the survival of mice vaccinated with B-MPP as described in the legend for FIG. 15 and challenged as described in the legend for FIG. 15 with 25×LD₅₀ of B/New York/PV01181/2018. FIG. 18C depicts the results from an ELISA measuring the binding of sera from mice immunized in accordance with the protocol provided in FIG. 15 and the legend for FIG. 15 for groups 1 to 3 to recombinant B-VASP (which comprises the globular head domain of influenza virus B/Colorado/06/2017 NA and the human VASP tetramerization domain).

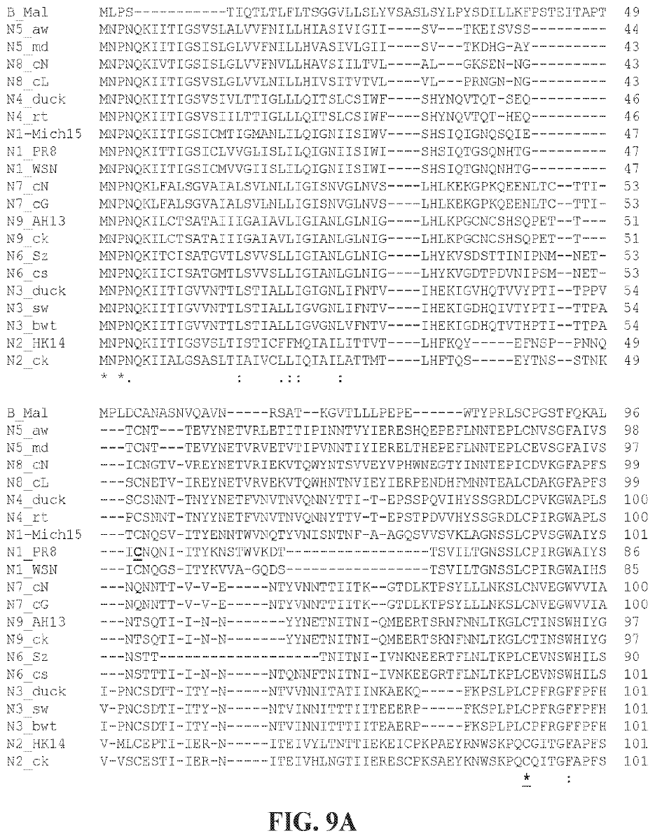

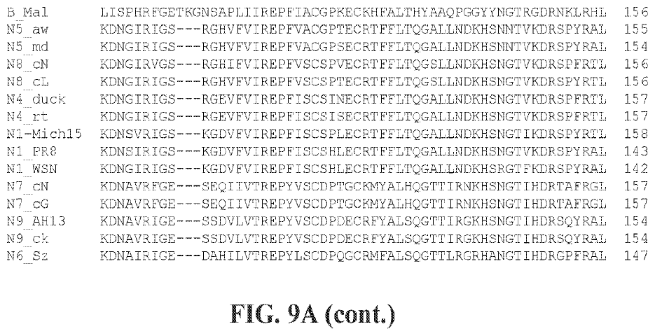

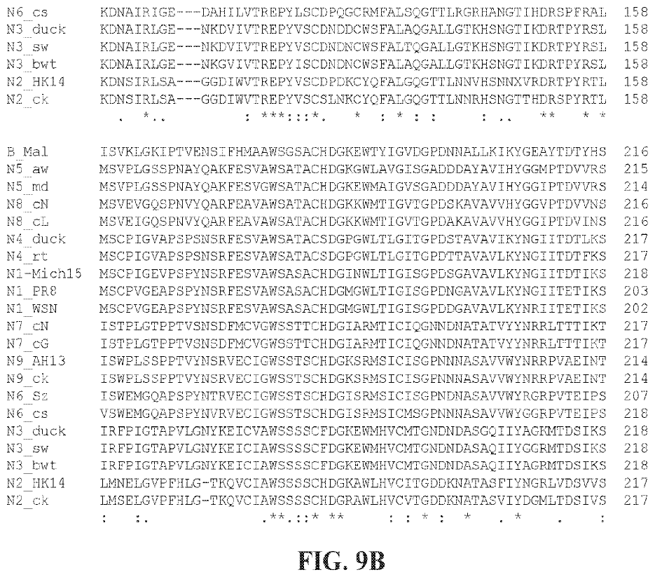

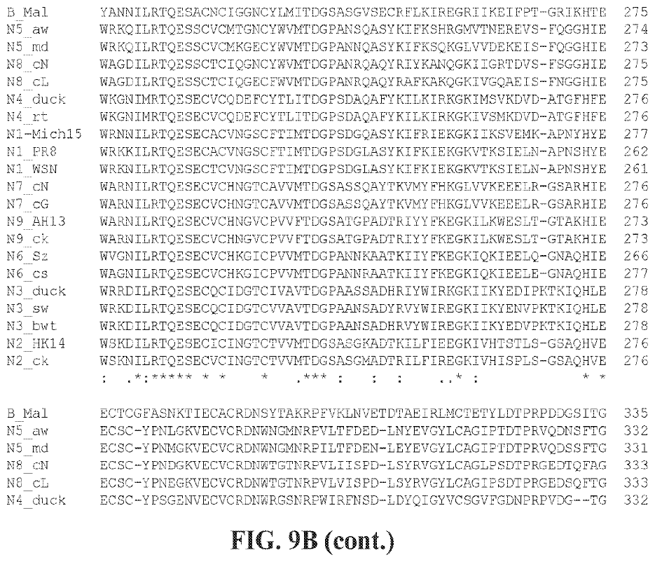

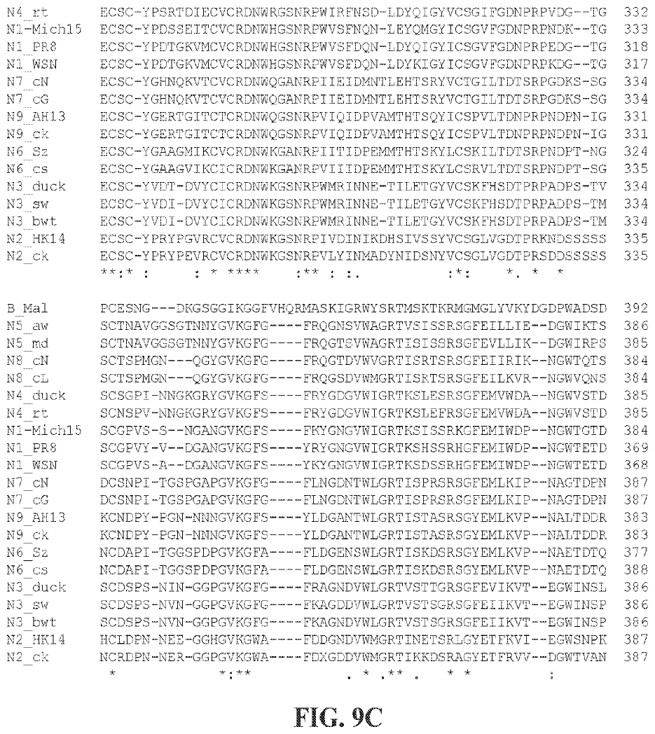

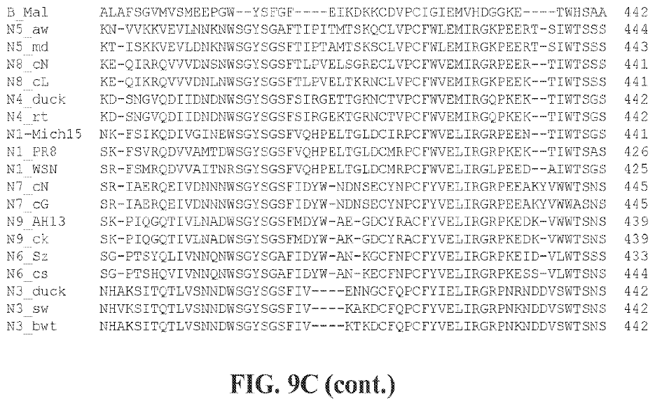

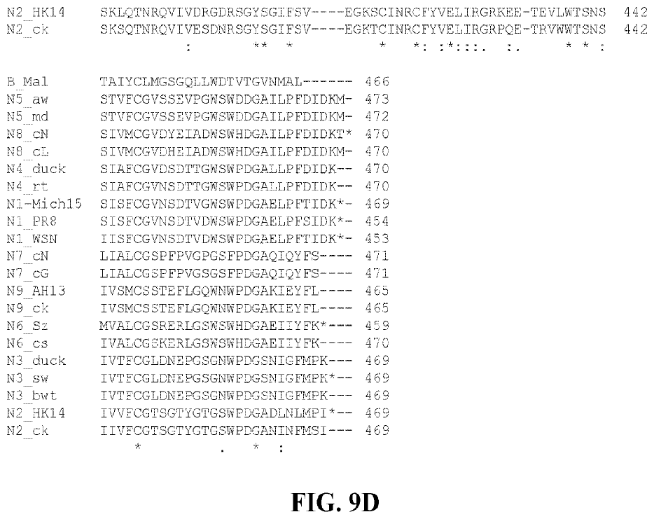

### 4.1 Sequence Information

| SEQ ID | | |
|---|---|---|
| NO. | NAME | SEQUENCE |
| 1 | Tetramerization | VELSSQQEYLKLKERYDALQRTQRNLLGEDLGPLSTKE |
| | domain from | LESLERQLDSSLKQIRALRTQFMLDQSKERMLTETNKT |
| | SEPPALLATA- | LRLRLADGY |
| | like MADS | |
| | domain | |
| | transcription | |
| | factor from | |
| | Arabidopsis | |
| | thaliana | |
| | (SMDTF) | |
| 2 | Sendai virus | ENTSSMKEMATLLTSLGVIQSAQEFESSRDASYVFARR |
| | phosphoprotein | ALKSANYAEMTFNVCGLILSAEKSSARKVDENKQLLKI |
| | tetramerization | QESVESFRDIYKRFSEYQKEQNSLLMSNLSTLHIITD |
| | domain (SPP) | |
| 3 | PiLZ structure | LLVQRMDAKLDLILALIGRLVRQS |
| | from | |
| | Xhantomonas | |
| | campestris | |
| 4 | Measles virus | GDHYDDELFSDVQDIKTALAKIHEDNQKIISKLESLLLL |
| | phosphoprotein | KGEVESIKKQFNRQNISISTLEGHLSSIMIAIPGL |
| | tetramerization | |
| | domain (MPP) | |
| 5 | Dictyocaulus | AVADVGDPFLLWKQQMDKWQNEYITDWQYHFEQYK |
| | viviparus ACE | KYQTYRHLDSDSCSGS |
| | tetramerization | |
| | domain | |
| 6 | PR8 N1 Wild- | MNPNQKITTIGSICLVVGLISLILQIGNIISIWISHSIQTGS |
| | type | QNHTGICNQNIITYKNSTWVKDTTSVILTGNSSLCPIRG |
| | | WAIYSKDNSIRIGSKGDVFVIREPFISCSHLECRTFFLTQ |
| | | GALLNDKHSNGTVKDRSPYRALMSCPVGEAPSPYNSR |
| | | FESVAWSASACHDGMGWLTIGISGPDNGAVAVLKYN |
| | | GIITETIKSWRKKILRTQESECACVNGSCFTIMTDGPSD |
| | | GLASYKIFKIEKGKVTKSIELNAPNSHYEECSCYPDTGK |
| | | VMCVCRDNWHGSNRPWVSFDQNLDYQIGYICSGVFG |
| | | DNPRPEDGTGSCGPVYVDGANGVKGFSYRYGNGVWI |
| | | GRTKSHSSRHGFEMIWDPNGWTETDSKFSVRQDVVA |
| | | MTDWSGYSGSFVQHPELTGLDCMRPCFWVELIRGRPK |
| | | EKTIWTSASSISFCGVNSDTVDWSWPDGAELPFSIDK* |
| 7 | PR8 N1 I48C | MNPNQKITTIGSICLVVGLISLILQIGNIISIWISHSIQTGS |
| | | QNHTGCCNQNIITYKNSTWVKDTTSVILTGNSSLCPIRG |
| | | WAIYSKDNSIRIGSKGDVFVIREPFISCSHLECRTFFLTQ |
| | | GALLNDKHSNGTVKDRSPYRALMSCPVGEAPSPYNSR |
| | | FESVAWSASACHDGMGWLTIGISGPDNGAVAVLKYN |
| | | GIITETIKSWRKKILRTQESECACVNGSCFTIMTDGPSD |
| | | GLASYKIFKIEKGKVTKSIELNAPNSHYEECSCYPDTGK |
| | | VMCVCRDNWHGSNRPWVSFDQNLDYQIGYICSGVFG |
| | | DNPRPEDGTGSCGPVYVDGANGVKGFSYRYGNGVWI |
| | | GRTKSHSSRHGFEMIWDPNGWTETDSKFSVRQDVVA |
| | | MTDWSGYSGSFVQHPELTGLDCMRPCFWVELIRGRPK |
| | | EKTIWTSASSISFCGVNSDTVDWSWPDGAELPFSIDK* |
| 8 | PR8 N1 N50C | MNPNQKITTIGSICLVVGLISLILQIGNIISIWISHSIQTGS |
| | | QNHTGICCQNIITYKNSTWVKDTTSVILTGNSSLCPIRG |
| | | WAIYSKDNSIRIGSKGDVFVIREPFISCSHLECRTFFLTQ |
| | | GALLNDKHSNGTVKDRSPYRALMSCPVGEAPSPYNSR |
| | | FESVAWSASACHDGMGWLTIGISGPDNGAVAVLKYN |
| | | GIITETIKSWRKKILRTQESECACVNGSCFTIMTDGPSD |
| | | GLASYKIFKIEKGKVTKSIELNAPNSHYEECSCYPDTGK |
| | | VMCVCRDNWHGSNRPWVSFDQNLDYQIGYICSGVFG |
| | | DNPRPEDGTGSCGPVYVDGANGVKGFSYRYGNGVWI |
| | | GRTKSHSSRHGFEMIWDPNGWTETDSKFSVRQDVVA |
| | | MTDWSGYSGSFVQHPELTGLDCMRPCFWVELIRGRPK |
| | | EKTIWTSASSISFCGVNSDTVDWSWPDGAELPFSIDK* |
| 9 | PR8 N1 61insertC | MNPNQKITTIGSICLVVGLISLILQIGNIISIWISHSIQTGS |
| | | QNHTGICNQNIITYKNSTWCVKDTTSVILTGNSSLCPIR |
| | | GWAIYSKDNSIRIGSKGDVFVIREPFISCSHLECRTFFLT |
| | | QGALLNDKHSNGTVKDRSPYRALMSCPVGEAPSPYNS |
| | | RFESVAWSASACHDGMGWLTIGISGPDNGAVAVLKYN |
| | | GIITETIKSWRKKILRTQESECACVNGSCFTIMTDGPSD |
| | | GLASYKIFKIEKGKVTKSIELNAPNSHYEECSCYPDTGK |
| | | VMCVCRDNWHGSNRPWVSFDQNLDYQIGYICSGVFG |
| | | DNPRPEDGTGSCGPVYVDGANGVKGFSYRYGNGVWI |
| | | GRTKSHSSRHGFEMIWDPNGWTETDSKFSVRQDVVA |
| | | MTDWSGYSGSFVQHPELTGLDCMRPCFWVELIRGRPK |
| | | EKTIWTSASSISFCGVNSDTVDWSWPDGAELPFSIDK* |
| 10 | PR8N1 | MNPNQKITTIGSICLVVGLISLILQIGNIISIWISHSIQTGS |
| | I48C_N50C | QNHTGCCCQNIITYKNSTWVKDTTSVILTGNSSLCPIRG |
| | | WAIYSKDNSIRIGSKGDVFVIREPFISCSHLECRTFFLTQ |
| | | GALLNDKHSNGTVKDRSPYRALMSCPVGEAPSPYNSR |
| | | FESVAWSASACHDGMGWLTIGISGPDNGAVAVLKYN |
| | | GIITETIKSWRKKILRTQESECACVNGSCFTIMTDGPSD |
| | | GLASYKIFKIEKGKVTKSIELNAPNSHYEECSCYPDTGK |
| | | VMCVCRDNWHGSNRPWVSFDQNLDYQIGYICSGVFG |
| | | DNPRPEDGTGSCGPVYVDGANGVKGFSYRYGNGVWI |
| | | GRTKSHSSRHGFEMIWDPNGWTETDSKFSVRQDVVA |
| | | MTDWSGYSGSFVQHPELTGLDCMRPCFWVELIRGRPK |
| | | EKTIWTSASSISFCGVNSDTVDWSWPDGAELPFSIDK* |
| 11 | PR8N1 | MNPNQKITTIGSICLVVGLISLILQIGNIISIWISHSIQTGS |
| | I48C_61insertC | QNHTGCCNQNIITYKNSTWCVKDTTSVILTGNSSLCPIR |
| | | GWAIYSKDNSIRIGSKGDVFVIREPFISCSHLECRTFFLT |
| | | QGALLNDKHSNGTVKDRSPYRALMSCPVGEAPSPYNS |
| | | RFESVAWSASACHDGMGWLTIGISGPDNGAVAVLKYN |
| | | GIITETIKSWRKKILRTQESECACVNGSCFTIMTDGPSD |
| | | GLASYKIFKIEKGKVTKSIELNAPNSHYEECSCYPDTGK |
| | | VMCVCRDNWHGSNRPWVSFDQNLDYQIGYICSGVFG |
| | | DNPRPEDGTGSCGPVYVDGANGVKGFSYRYGNGVWI |
| | | GRTKSHSSRHGFEMIWDPNGWTETDSKFSVRQDVVA |
| | | MTDWSGYSGSFVQHPELTGLDCMRPCFWVELIRGRPK |
| | | EKTIWTSASSISFCGVNSDTVDWSWPDGAELPFSIDK* |
| 12 | HK14 N2 Wild- | MNPNQKIITIGSVSLTISTICFFMQIAILITTVTLHFKQYE |
| | type | FNSPPNNQVMLCEPTIIERNITEIVYLTNTTIEKEICPKPA |
| | | EYRNWSKPQCGITGFAPFSKDNSIRLSAGGDIWVTREP |
| | | YVSCDPDKCYQFALGQGTTLNNVHSNNTVRDRTPYRT |
| | | LLMNELGVPFHLGTKQVCIAWSSSSCHDGKAWLHVCI |
| | | TGDDKNATASFIYNGRLVDSVVSWSKDILRTQESECICI |
| | | NGTCTVVMTDGSASGKADTKILFIEEGKIVHTSTLSGSA |
| | | QHVEECSCYPRYPGVRCVCRDNWKGSNRPIVDINIKDH |
| | | SIVSSYVCSGLVGDTPRKNDSSSSSHCLDPNNEEGGHG |
| | | VKGWAFDDGNDVWMGRTINETSRLGYETFKVIEGWS |
| | | NPKSKLQTNRQVIVDRGDRSGYSGIFSVEGKSCINRCFY |
| | | VELIRGRKEETEVLWTSNSIVVFCGTSGTYGTGSWPDG |
| | | ADLNLMPI* |
| 13 | HK14 N2 L52C | MNPNQKIITIGSVSLTISTICFFMQIAILITTVTLHFKQYE |
| | | FNSPPNNQVMCCEPTIIERNITEIVYLTNTTIEKEICPKPA |
| | | EYRNWSKPQCGITGFAPFSKDNSIRLSAGGDIWVTREP |
| | | YVSCDPDKCYQFALGQGTTLNNVHSNNTVRDRTPYRT |
| | | LLMNELGVPFHLGTKQVCIAWSSSSCHDGKAWLHVCI |
| | | TGDDKNATASFIYNGRLVDSVVSWSKDILRTQESECICI |
| | | NGTCTVVMTDGSASGKADTKILFIEEGKIVHTSTLSGSA |
| | | QHVEECSCYPRYPGVRCVCRDNWKGSNRPIVDINIKDH |
| | | SIVSSYVCSGLVGDTPRKNDSSSSSHCLDPNNEEGGHG |
| | | VKGWAFDDGNDVWMGRTINETSRLGYETFKVIEGWS |
| | | NPKSKLQTNRQVIVDRGDRSGYSGIFSVEGKSCINRCFY |
| | | VELIRGRKEETEVLWTSNSIVVFCGTSGTYGTGSWPDG |
| | | ADLNLMPI* |
| 14 | HK14 N2 | MNPNQKIITIGSVSLTISTICFFMQIAILITTVTLHFKQYE |
| | L52C_E54C | FNSPPNNQVMCCCPTIIERNITEIVYLTNTTIEKEICPKPA |
| | | EYRNWSKPQCGITGFAPFSKDNSIRLSAGGDIWVTREP |
| | | YVSCDPDKCYQFALGQGTTLNNVHSNNTVRDRTPYRT |
| | | LLMNELGVPFHLGTKQVCIAWSSSSCHDGKAWLHVCI |
| | | TGDDKNATASFIYNGRLVDSVVSWSKDILRTQESECICI |
| | | NGTCTVVMTDGSASGKADTKILFIEEGKIVHTSTLSGSA |
| | | QHVEECSCYPRYPGVRCVCRDNWKGSNRPIVDINIKDH |
| | | SIVSSYVCSGLVGDTPRKNDSSSSSHCLDPNNEEGGHG |
| | | VKGWAFDDGNDVWMGRTINETSRLGYETFKVIEGWS |
| | | NPKSKLQTNRQVIVDRGDRSGYSGIFSVEGKSCINRCFY |
| | | VELIRGRKEETEVLWTSNSIVVFCGTSGTYGTGSWPDG |
| | | ADLNLMPI* |
| 15 | N1.2 | MLLVNQSHQGFNKEHTSKMVSAIVLYVLLAAAAHSAF |
| | | AADPHHHHHHSLVPRGSPSRIETCNQSVITYENNTWVN |
| | | QTYVNISNTNFAAGQSVVSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWT |
| | | SGSSISFCGVNSDTVGWSWPDGAELPFTIDK* |
| 16 | N1.3 (C49A- | MLLVNQSHQGFNKEHTSKMVSAIVLYVLLAAAAHSAF |
| | monomer) | AADPHHHHHHSLVPRGSPSRIETANQSVITYENNTWVN |
| | | QTYVNISNTNFAAGQSVVSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWT |
| | | SGSSISFCGVNSDTVGWSWPDGAELPFTIDK* |
| 17 | N1.4 (T48C) | MLLVNQSHQGFNKEHTSKMVSAIVLYVLLAAAAHSAF |
| | | AADPHHHHHHSLVPRGSPSRIECCNQSVITYENNTWVN |
| | | QTYVNISNTNFAAGQSVVSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWTSGSSISFCGV |
| | | NSDTVGWSWPDGAELPFTIDK* |
| 18 | N1.5 (N50C) | MLLVNQSHQGFNKEHTSKMVSAIVLYVLLAAAAHSAF |
| | | AADPHHHHHHSLVPRGSPSRIETCCQSVITYENNTWVN |
| | | QTYVNISNTNFAAGQSVVSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWT |
| | | SGSSISFCGVNSDTVGWSWPDGAELPFTIDK* |
| 19 | N1.6 | MLLVNQSHQGFNKEHTSKMVSAIVLYVLLAAAAHSAF |
| | (T48C + N50C) | AADPHHHHHHSLVPRGSPSRIECCCQSVITYENNTWVN |
| | | QTYVNISNTNFAAGQSVVSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWTSGSSISFCGV |
| | | NSDTVGWSWPDGAELPFTIDK* |
| 20 | N1.7(A76C) | MLLVNQSHQGFNKEHTSKMVSAIVLYVLLAAAAHSAF |
| | | AADPHHHHHHSLVPRGSPSRIETCNQSVITYENNTWVN |
| | | QTYVNISNTNFACGQSVVSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWTSGSSISFCGV |
| | | NSDTVGWSWPDGAELPFTIDK* |
| 21 | N1.8 (Q78C) | MLLVNQSHQGFNKEHTSKMVSAIVLYVLLAAAAHSAF |
| | | AADPHHHHHHSLVPRGSPSRIETCNQSVITYENNTWVN |
| | | QTYVNISNTNFAAGCSVVSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWTSGSSISFCGV |
| | | NSDTVGWSWPDGAELPFTIDK* |
| 22 | N1.9 (V81C) | MLLVNQSHQGFNKEHTSKMVSAIVLYVLLAAAAHSAF |
| | | AADPHHHHHHSLVPRGSPSRIETCNQSVITYENNTWVN |
| | | QTYVNISNTNFAAGQSVCSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWTSGSSISFCGV |
| | | NSDTVGWSWPDGAELPFTIDK* |
| 23 | N1.11 (W61C) | MLLVNQSHQGFNKEHTSKMVSAIVLYVLLAAAAHSAF |
| | | AADPHHHHHHSLVPRGSPSRIETCNQSVITYENNTCVN |
| | | QTYVNISNTNFAAGQSVVSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWTSGSSISFCGV |
| | | NSDTVGWSWPDGAELPFTIDK* |
| 24 | SMDTF-N1 | MLLVNQSHQGFNKEHTSKMVSAIVLYVLLAAAAHSAF |
| | | AADPHHHHHHVELSSQQEYLKLKERYDALQRTQRNLL |
| | | GEDLGPLSTKELESLERQLDSSLKQIRALRTQFMLDQS |
| | | KERMLTETNKTLRLRLADGYSLVPRGSPSRSVKLAGNS |
| | | SLCPVSGWAIYSKDNSVRIGSKGDVFVIREPFISCSPLEC |
| | | RTFFLTQGALLNDKHSNGTIKDRSPYRTLMSCPIGEVPS |
| | | PYNSRFESVAWSASACHDGINWLTIGISGPDSGAVAVL |
| | | KYNGIITDTIKSWRNNILRTQESECACVNGSCFTIMTDG |
| | | PSDGQASYKIFRIEKGKIIKSVEMKAPNYHYEECSCYPD |
| | | SSEITCVCRDNWHGSNRPWVSFNQNLEYQMGYICSGV |
| | | FGDNPRPNDKTGSCGPVSSNGANGVKGFSFKYGNGV |
| | | WIGRTKSISSRKGFEMIWDPNGWTGTDNKFSIKQDIVGI |
| | | NEWSGYSGSFVQHPELTGLDCIRPCFWVELIRGRPEEN |
| | | TIWTSGSSISFCGVNSDTVGWSWPDGAELPFTIDK* |
| 25 | SPP-N1 | MLLVNQSHQGFNKEHTSKMVSAIVLYVLLAAAAHSAF |
| | | AADPHHHHHHENTSSMKEMATLLTSLGVIQSAQEFESS |
| | | RDASYVFARRALKSANYAEMTFNVCGLILSAEKSSAR |
| | | KVDENKQLLKIQESVESFRDIYKRFSEYQKEQNSLLMS |
| | | NLSTLHIITDSLVPRGSPSRSVKLAGNSSLCPVSGWAIYS |
| | | KDNSVRIGSKGDVFVIREPFISCSPLECRTFFLTQGALLN |
| | | DKHSNGTIKDRSPYRTLMSCPIGEVPSPYNSRFESVAWS |
| | | ASACHDGINWLTIGISGPDSGAVAVLKYNGIITDTIKSW |
| | | RNNILRTQESECACVNGSCFTIMTDGPSDGQASYKIFRI |
| | | EKGKIIKSVEMKAPNYHYEECSCYPDSSEITCVCRDNW |
| | | HGSNRPWVSFNQNLEYQMGYICSGVFGDNPRPNDKTG |
| | | SCGPVSSNGANGVKGFSFKYGNGVWIGRTKSISSRKGF |
| | | EMIWDPNGWTGTDNKFSIKQDIVGINEWSGYSGSFVQ |
| | | HPELTGLDCIRPCFWVELIRGRPEENTIWTSGSSISFCGV |
| | | NSDTVGWSWPDGAELPFTIDK* |
| 26 | PiLZ-Nl | MLLVNQSHQGFNKEHTSKMVSAIVLYVLLAAAAHSAF |
| | | AADPHHHHHHLLVQRMDAKLDLILALIGRLVRQSSLV |
| | | PRGSPSRSVKLAGNSSLCPVSGWAIYSKDNSVRIGSKG |
| | | DVFVIREPFISCSPLECRTFFLTQGALLNDKHSNGTIKDR |
| | | SPYRTLMSCPIGEVPSPYNSRFESVAWSASACHDGINW |
| | | LTIGISGPDSGAVAVLKYNGIITDTIKSWRNNILRTQESE |
| | | CACVNGSCFTIMTDGPSDGQASYKIFRIEKGKIIKSVEM |
| | | KAPNYHYEECSCYPDSSEITCVCRDNWHGSNRPWVSF |
| | | NQNLEYQMGYICSGVFGDNPRPNDKTGSCGPVSSNGA |
| | | NGVKGFSFKYGNGVWIGRTKSISSRKGFEMIWDPNGW |
| | | TGTDNKFSIKQDIVGFNEWSGYSGSFVQHPELTGLDCIR |
| | | PCFWVELIRGRPEENTIWTSGSSISFCGVNSDTVGWSW |
| | | PDGAELPFTIDK* |
| 27 | MPP-N1 | MLLVNQSHQGFNKEHTSKMVSAIVLYVLLAAAAHSAF |
| | | AADPHHHHHHGDHYDDELFSDVQDIKTALAKIHEDNQ |
| | | KIISKLESLLLLKGEVESIKKQINRQNISISTLEGHLSSIMI |
| | | AIPGLSLVPRGSPSRSVKLAGNSSLCPVSGWAIYSKDNS |
| | | VRIGSKGDVFVIREPFISCSPLECRTFFLTQGALLNDKHS |
| | | NGTIKDRSPYRTLMSCPIGEVPSPYNSRFESVAWSASAC |
| | | HDGINWLTIGISGPDSGAVAVLKYNGIITDTIKSWRNNI |
| | | LRTQESECACVNGSCFTIMTDGPSDGQASYKIFRIEKGK |
| | | IIKSVEMKAPNYHYEECSCYPDSSEITCVCRDNWHGSN |
| | | RPWVSFNQNLEYQMGYICSGVFGDNPRPNDKTGSCGP |
| | | VSSNGANGVKGFSFKYGNGVWIGRTKSISSRKGFEMI |
| | | WDPNGWTGTDNKFSIKQDIVGINEWSGYSGSFVQHPE |
| | | LTGLDCIRPCFWVELIRGRPEENTIWTSGSSISFCGVNS |
| | | DTVGWSWPDGAELPFTIDK* |
| 28 | ACE-N1 | MLLVNQSHQGFNKEHTSKMVSAFVLYVLLAAAAHSAF |
| | | AADPHHHHHHAVADVGDPFLLWKQQMDKWQNEYIT |
| | | DWQYHFEQYKKYQTYRHLDSDSCSGSSLVPRGSPSRS |
| | | VKLAGNSSLCPVSGWAIYSKDNSVRIGSKGDVFVIREP |
| | | FISCSPLECRTFFLTQGALLNDKHSNGTIKDRSPYRTLM |
| | | SCPIGEVPSPYNSRFESVAWSASACHDGINWLTIGISGP |
| | | DSGAVAVLKYNGIITDTIKSWRNNILRTQESECACVNG |
| | | SCFTIMTDGPSDGQASYKIFRIEKGKIIKSVEMKAPNYH |
| | | YEECSCYPDSSEITCVCRDNWHGSNRPWVSFNQNLEY |
| | | QMGYICSGVFGDNPRPNDKTGSCGPVSSNGANGVKGF |
| | | SFKYGNGVWIGRTKSISSRKGFEMIWDPNGWTGTDNK |
| | | FSIKQDIVGINEWSGYSGSFVQHPELTGLDCIRPCFWVE |
| | | LIRGRPEENTIWTSGSSISFCGVNSDTVGWSWPDGAELP |
| | | FTIDK* |
| 29-49 | Influenza virus | See FIGS. 9A-9D |
| | neuraminidase of | |
| | different strains | |
| 50 | His tag | HHHHHH |
| 51 | Cleavage site | LVPRGSP |
| 52 | Cleavage site | ENLYFQX (X = G/S) |
| 53 | PR8N1 (aa 1- | MNPNQKITTIGSICLVVGLISLILQIGNIISIWISHSIQTGS |
| | 102) | QNHTGICNQNIITYKNSTWVKDTTSVILTGNSSLCPIRG |
| | | WAIYSKDNSIRIGSKGDVFVI |
| 54 | HK14 N2 (aa 1- | MNPNQKIITIGSVSLTISTICFFMQIAILITTVTLHFKQYE |
| | 117) | FNSPPNNQVMLCEPTIIERNITEIVYLTNTTIEKEICPKPA |
| | | EYRNWSKPQCGITGFAPFSKDNSIRLSAGGDIWVT |
| 55 | N1-MPP (with his | atgctgctcgtcaaccaatcccaccagggcttcaacaaggaacacacttctaagatggt |
| | tag) | ctccgctatcgtgctctacgtgctgctcgctgccgctgcccactcagctttcgctgccga |
| | | cccacaccaccaccaccaccacggcgatcactacgacgacgaactgttctccgacgt |
| | | gcaggacatcaagaccgctctggctaagatccacgaggacaaccagaagatcatctc |
| | | caagctggaatccctgctgctgctgaagggcgaagtcgagtccatcaagaagcagatc |
| | | aaccgccagaacatctccatctccaccttggagggtcacctgtcctccatcatgatcgct |
| | | atccctggcctgGGCGGCGGCTCCGTGAAATTAGCGGGCA |
| | | ATTCCTCTCTCTGCCCTGTTAGTGGATGGGCTATATA |
| | | CAGTAAAGACAACAGTGTAAGAATCGGTTCCAAGGG |
| | | GGATGTGTTTGTCATAAGGGAACCATTCATATCATG |
| | | CTCTCCCTTGGAATGCAGAACCTTCTTCTTGACTCAA |
| | | GGGGCCTTGCTAAATGACAAACATTCCAATGGAACC |
| | | ATTAAAGACAGGAGCCCATACCGAACCCTAATGAGC |
| | | TGTCCTATTGGTGAAGTTCCCTCTCCATACAACTCAA |
| | | GATTTGAGTCAGTCGCTTGGTCAGCAAGTGCTTGTCA |
| | | TGATGGCATCAATTGGCTAACAATTGGAATTTCTGG |
| | | CCCAGACAGTGGGGCAGTGGCTGTGTTAAAGTACAA |
| | | TGGCATAATAACAGACACTATCAAGAGTTGGAGGAA |
| | | CAATATATTGAGAACACAAGAGTCTGAATGTGCATG |
| | | TGTAAATGGTTCTTGCTTTACCATAATGACCGATGGA |
| | | CCAAGTGATGGACAGGCCTCATACAAAATCTTCAGA |
| | | ATAGAAAAGGGAAAGATAATCAAATCAGTCGAAAT |
| | | GAAAGCCCCTAATTATCACTATGAGGAATGCTCCTG |
| | | TTACCCTGATTCTAGTGAAATCACATGTGTGTGCAGG |
| | | GATAACTGGCATGGCTCGAATCGACCGTGGGTGTCT |
| | | TTCAACCAGAATCTGGAATATCAGATGGGATACATA |
| | | TGCAGTGGGGTTTTCGGAGACAATCCACGCCCTAAT |
| | | GATAAGACAGGCAGTTGTGGTCCAGTATCGTCTAAT |
| | | GGAGCAAATGGAGTAAAAGGATTTTCATTCAAATAC |
| | | GGCAATGGTGTTTGGATAGGGAGAACTAAAAGCATT |
| | | AGTTCAAGAAAAGGTTTTGAGATGATTTGGGATCCG |
| | | AATGGATGGACTGGGACTGACAATAAATTCTCAATA |
| | | AAGCAAGATATCGTAGGAATAAATGAGTGGTCAGG |
| | | GTATAGCGGGAGTTTTGTTCAGCATCCAGAACTAAC |
| | | AGGGCTGGATTGTATAAGACCTTGCTTCTGGGTTGA |
| | | ACTAATAAGAGGGCGACCCGAAGAGAACACAATCT |
| | | GGACTAGCGGGAGCAGCATATCCTTTTGTGGTGTAA |
| | | ACAGTGACACTGTGGGTTGGTCTTGGCCAGACGGTG |
| | | CTGAGTTGCCATTTACCATTGACAAGTAA |
| 56 | N1-MPP (with his | MLLVNQSHQGFNKEHTSKMVSAIVLYVLLAAAAHSAF |
| | tag) | AADPHHHHHHGDHYDDELFSDVQDIKTALAKIHEDNQ |
| | | KIISKLESLLLLKGEVESIKKQINRQNISISTLEGHLSSIMI |
| | | AIPGLGGGSVKLAGNSSLCPVSGWAIYSKDNSVRIGSK |
| | | GDVFVIREPFISCSPLECRTFFLTQGALLNDKHSNGTIKD |
| | | RSPYRTLMSCPIGEVPSPYNSRFESVAWSASACHDGIN |
| | | WLTIGISGPDSGAVAVLKYNGIITDTIKSWRNNILRTQE |
| | | SECACVNGSCFTIMTDGPSDGOASYKIFRIEKGKIIKSVE |
| | | MKAPNYHYEECSCYPDSSEITCVCRDNWHGSNRPWVS |
| | | FNQNLEYQMGYICSGVFGDNPRPNDKTGSCGPVSSNG |
| | | ANGVKGFSFKYGNGVWIGRTKSISSRKGFEMIWDPNG |
| | | WTGTDNKFSIKQDIVGINEWSGYSGSFVQHPELTGLDC |
| | | IRPCFWVELIRGRPEENTIWTSGSSISFCGVNSDTVGWS |
| | | WPDGAELPFTIDK* |
| 57 | N1-MPP (no his | atgctgctcgtcaaccaatcccaccagggcttcaacaaggaacacacttctaagatggt |
| | tag) | ctccgctatcgtgctctacgtgctgctcgctgccgctgcccactcagctttcgctgccga |
| | | cccaggcgatcactacgacgacgaactgttctccgacgtgcaggacatcaagaccgct |
| | | ctggctaagatccacgaggacaaccagaagatcatctccaagctggaatccctgctgc |
| | | tgctgaagggcgaagtcgagtccatcaagaagcagatcaaccgccagaacatctccat |
| | | ctccaccttggagggtcacctgtcctccatcatgatcgctatccctggcctgGGCGG |
| | | CGGCTCCGTGAAATTAGCGGGCAATTCCTCTCTCTGC |
| | | CCTGTTAGTGGATGGGCTATATACAGTAAAGACAAC |
| | | AGTGTAAGAATCGGTTCCAAGGGGGATGTGTTTGTC |
| | | ATAAGGGAACCATTCATATCATGCTCTCCCTTGGAAT |
| | | GCAGAACCTTCTTCTTGACTCAAGGGGCCTTGCTAA |
| | | ATGACAAACATTCCAATGGAACCATTAAAGACAGGA |
| | | GCCCATACCGAACCCTAATGAGCTGTCCTATTGGTG |
| | | AAGTTCCCTCTCCATACAACTCAAGATTTGAGTCAGT |
| | | CGCTTGGTCAGCAAGTGCTTGTCATGATGGCATCAA |
| | | TTGGCTAACAATTGGAATTTCTGGCCCAGACAGTGG |
| | | GGCAGTGGCTGTGTTAAAGTACAATGGCATAATAAC |
| | | AGACACTATCAAGAGTTGGAGGAACAATATATTGAG |
| | | AACACAAGAGTCTGAATGTGCATGTGTAAATGGTTC |
| | | TTGCTTTACCATAATGACCGATGGACCAAGTGATGG |
| | | ACAGGCCTCATACAAAATCTTCAGAATAGAAAAGGG |
| | | AAAGATAATCAAATCAGTCGAAATGAAAGCCCCTAA |
| | | TTATCACTATGAGGAATGCTCCTGTTACCCTGATTCT |
| | | AGTGAAATCACATGTGTGTGCAGGGATAACTGGCAT |
| | | GGCTCGAATCGACCGTGGGTGTCTTTCAACCAGAAT |
| | | CTGGAATATCAGATGGGATACATATGCAGTGGGGTT |
| | | TTCGGAGACAATCCACGCCCTAATGATAAGACAGGC |
| | | AGTTGTGGTCCAGTATCGTCTAATGGAGCAAATGGA |
| | | GTAAAAGGATTTTCATTCAAATACGGCAATGGTGTT |
| | | TGGATAGGGAGAACTAAAAGCATTAGTTCAAGAAA |
| | | AGGTTTTGAGATGATTTGGGATCCGAATGGATGGAC |
| | | TGGGACTGACAATAAATTCTCAATAAAGCAAGATAT |
| | | CGTAGGAATAAATGAGTGGTCAGGGTATAGCGGGA |
| | | GTTTTGTTCAGCATCCAGAACTAACAGGGCTGGATT |
| | | GTATAAGACCTTGCTTCTGGGTTGAACTAATAAGAG |
| | | GGCGACCCGAAGAGAACACAATCTGGACTAGCGGG |
| | | AGCAGCATATCCTTTTGTGGTGTAAACAGTGACACT |
| | | GTGGGTTGGTCTTGGCCAGACGGTGCTGAGTTGCCA |
| | | TTTACCATTGACAAGTAA |
| 58 | N1-MPP (no his | MLLVNQSHQGFNKEHTSKMVSAIVLYVLLAAAAHSAF |
| | tag) | AADPGDHYDDELFSDVQDIKTALAKIHEDNQKIISKLES |
| | | LLLLKGEVESIKKQINRQNISISTLEGHLSSIMIAIPGLGG |
| | | GSVKLAGNSSLCPVSGWAIYSKDNSVRIGSKGDVFVIR |
| | | EPFISCSPLECRTFFLTQGALLNDKHSNGTIKDRSPYRTL |
| | | MSCPIGEVPSPYNSRFESVAWSASACHDGINWLTIGISG |
| | | PDSGAVAVLKYNGIITDTIKSWRNNILRTQESECACVN |
| | | GSCFTIMTDGPSDGQASYKIFRIEKGKIIKSVEMKAPNY |
| | | HYEECSCYPDSSEITCVCRDNWHGSNRPWVSFNQNLE |
| | | YQMGYICSGVFGDNPRPNDKTGSCGPVSSNGANGVKG |
| | | FSFKYGNGVWIGRTKSISSRKGFEMIWDPNGWTGTDN |
| | | KFSIKQDIVGINEWSGYSGSFVQHPELTGLDCIRPCFWV |
| | | ELIRGRPEENTIWTSGSSISFCGVNSDTVGWSWPDGAEL |
| | | PFTIDK* |
| 59 | N2-MPP | atgctgctcgtcaaccaatcccaccagggcttcaacaaggaacacacttctaagatggt |
| | | ctccgctatcgtgctctacgtgctgctcgctgccgctgcccactcagctttcgctgccga |
| | | cccacaccaccaccaccaccacggcgatcactacgacgacgaactgttctccgacgt |
| | | gcaggacatcaagaccgctctggctaagatccacgaggacaaccagaagatcatctc |
| | | caagctggaatccctgctgctgctgaagggcgaagtcgagtccatcaagaagcagatc |
| | | aaccgccagaacatctccatctccaccttggagggtcacctgtcctccatcatgatcgct |
| | | atccctggcctgtctctcgtgcccaggggatcaccttctagaATATGCCCCAA |
| | | ACCAGCAGAATACAGAAATTGGTCAAAACCGCAATG |
| | | TGGCATTACAGGATTTGCACCTTTCTCTAAGGACAAT |
| | | TCGATTAGGCTTTCCGCTGGTGGGGACATCTGGGTG |
| | | ACAAGAGAACCTTATGTGTCATGCGATCCTGACAAG |
| | | TGTTATCAATTTGCCCTTGGACAGGGAACAACAATA |
| | | AACAACGTGCATTCAAATAACACAGCACGTGATAGG |
| | | ACCCCTCATCGGACTCTATTGATGAATGAGTTGGGT |
| | | GTTCCTTTCCATCTGGGGACCAAGCAAGTGTGCATA |
| | | GCATGGTCCAGCTCAAGTTGTCACGATGGAAAAGCA |
| | | TGGCTGCATGTTTGTATAACGGGGGATGATAAAAAT |
| | | GCAACTGCTAGTTTCATTTACAATGGGAGGCTTGTA |
| | | GATAGTGTTGTTTCATGGTCCAAAGATATTCTCAGGA |
| | | CCCAGGAGTCAGAATGCGTTTGTATCAATGGAACTT |
| | | GTACAGTAGTAATGACTGATGGAAATGCTACAGGAA |
| | | AAGCTGATACTAAAATATTATTCATTGAGGAGGGGA |
| | | AAATCGTTCATACTAGCAAATTGTCAGGAAGTGCTC |
| | | AGCATGTCGAAGAGTGCTCTTGCTATCCTCGATACCC |
| | | TGGTGTCAGATGTGTCTGCAGAGACAACTGGAAAGG |
| | | ATCCAACCGGCCCATCGTAGATATAAACATAAAGGA |
| | | TCATAGCATTGTTTCCAGTTATGTGTGTTCAGGACTT |
| | | GTTGGAGACACACCCAGAAAAACCGACAGCTCCAGC |
| | | AGCAGCCATTGCTTGAATCCTAACAATGAAAAAGGT |
| | | GGTCATGGAGTGAAAGGCTGGGCCTTTGATGATGGA |
| | | AATGACGTGTGGATGGGGAGAACAATCAACGAGAC |
| | | GTCACGCTTAGGGTATGAAACCTTCAAAGTCGTTGA |
| | | AGGCTGGTCCAACCCTAAGTCCAAATTGCAGATAAA |
| | | TAGGCAAGTCATAGTTGACAGAGGTGATAGGTCCGG |
| | | TTATTCTGGTATTTTCTCTGTTGAAGGCAAAAGCTGC |
| | | ATCAATCGGTGCTTTTATGTGGAGTTGATTAGGGGA |
| | | AGAAAAGAGGAAACTGAAGTCTTGTGGACCTCAAAC |
| | | AGTATTGTTGTGTTTTGTGGCACCTCAGGTACATATG |
| | | GAACAGGCTCATGGCCTGATGGGGCGGACCTCAATC |
| | | TCATGCATATATAA |
| 60 | N2-MPP | MLLVNQSHQGFNKEHTSKMVSAIVLYVLLAAAAHSAF |
| | | AADPHHHHHHGDHYDDELFSDVQDIKTALAKIHEDNQ |
| | | KIISKLESLLLLKGEVESIKKQINRQNISISTLEGHLSSIMI |
| | | AIPGLSLVPRGSPSRICPKPAEYRNWSKPQCGITGFAPFS |
| | | KDNSIRLSAGGDIWVTREPYVSCDPDKCYQFALGQGTT |
| | | INNVHSNNTARDRTPHRTLLMNELGVPFHLGTKQVCIA |
| | | WSSSSCHDGKAWLHVCITGDDKNATASFIYNGRLVDs |
| | | VVSWSKDILRTQESECVCINGTCTVVMTDGNATGKAD |
| | | TKILFIEEGKIVHTSKLSGSAQHVEECSCYPRYPGVRCV |
| | | CRDNWKGSNRPIVDINIKDHSIVSSYVCSGLVGDTPRK |
| | | TDSSSSSHCLNPNNEKGGHGVKGWAFDDGNDVWMGR |
| | | TINETSRLGYETFKVVEGWSNPKSKLQINRQVIVDRGD |
| | | RSGYSGIFSVEGKSCINRCFYVELIRGRKEETEVLWTSN |
| | | SIVVFCGTSGTYGTGSWPDGADLNLMHI* |
| 61 | B-MPP | atgctgctcgtcaaccaatcccaccagggcttcaacaaggaacacacttctaagatggt |
| | | ctccgctatcgtgctctacgtgctgctcgctgccgctgcccactcagctttcgctgccga |
| | | cccacaccaccaccaccaccacggcgatcactacgacgacgaactgttctccgacgt |
| | | gcaggacatcaagaccgctctggctaagatccacgaggacaaccagaagatcatctc |
| | | caagctggaatccctgctgctgctgaagggcgaagtcgagtccatcaagaagcagatc |
| | | aaccgccagaacatctccatctccaccttggagggtcacctgtcctccatcatgatcgct |
| | | atccctggcctgtctctcgtgcccaggggatcaccttctagacttcttctcccagaaccg |
| | | gagtggacatacccgcgtttatcttgcccgggctcaacctttcagaaagcactcctaatt |
| | | agccctcatagattcggagaaaccaaaggaaactcagctcccttgataataagggaac |
| | | cttttgttgcttgtggaccaaatgaatgcaaacactttgctttaacccattatgcagcccaa |
| | | ccagggggatactacaatggaacaagaggagacagaaacaagctgaggcatctaatt |
| | | tcagtcaaattgggcaaaatcccaacagtagagaactccattttccacatggcagcatg |
| | | gagcgggtccgcgtgccatgatggtaaggaatggacatatatcggagttgatggccct |
| | | gacaataatgcattgctcaaagtaaaatatggagaagcatatactgacacataccattcc |
| | | tatgcaaacaacatcctaagaacacaagaaagtgcctgcaattgcatcgggggaaatt |
| | | gttatctaatgataactgatggctcagcttcaggtgttagtgaatgcagatttcttaagattc |
| | | gagagggccgaataataaaagaaatatttccaacaggaagagtaaaacacactgagg |
| | | aatgcacatgcggatttgccagcaataaaaccatagaatgtgcctgtagagacaacag |
| | | gtacacagcaaaaagaccttttgtcaaattaaacgtggagactgatacagcagaaataa |
| | | ggttgatgtgcacagatacctatttggacacccccagaccaaatgatggaagcataaca |
| | | ggcccttgtgaatctgatggggacaaagggagtggaggcatcaagggaggatttgttc |
| | | atcaaagaatgaaatccaagattggaaggtggtactctcgaacgatgtctcaaactgaa |
| | | aggatggggatgggactgtatgtcaagtatggtggagacccatgggctgacagtgatg |
| | | ccctagcttttagtggagtaatggtttcaatgaaagaacctggttggtattcctttggcttcg |
| | | aaataaaagataagaaatgcgatgtcccctgtattgggatagagatggtacatgatggt |
| | | ggaaaagagacttggcactcagcagcaacagccatttactgtttaatgggctcaggaca |
| | | gctgctgtgggacactgtcacaggtgttgacatggctctgtaa |
| 62 | B-MPP | MLLVNQSHQGFNKEHTSKMVSAFVLYVLLAAAAHSAF |
| | | AADPHHHHHHGDHYDDELFSDVQDIKTALAKIHEDNQ |
| | | KIISKLESLLLLKGEVESIKKQINRQNISISTLEGHLSSIMI |
| | | AIPGLSLVPRGSPSRLLLPEPEWTYPRLSCPGSTFQKALL |
| | | ISPHRFGETKGNSAPLIIREPFVACGPNECKHFALTHYA |
| | | AQPGGYYNGTRGDRNKLRHLISVKLGKIPTVENSIFHM |
| | | AAWSGSACHDGKEWTYIGVDGPDNNALLKVKYGEAY |
| | | TDTYHSYANNILRTQESACNCIGGNCYLMITDGSASGV |
| | | SECRFLKIREGRIIKEIFPTGRVKHTEECTCGFASNKTIEC |
| | | ACRDNRYTAKRPFVKLNVETDTAEIRLMCTDTYLDTP |
| | | RPNDGSITGPCESDGDKGSGGIKGGFVHQRMKSKIGR |
| | | WYSRTMSQTERMGMGLYVKYGGDPWADSDALAFSG |
| | | VMVSMKEPGWYSFGFEIKDKKCDVPCIGIEMVHDGGK |
| | | ETWHSAATAIYCLMGSGQLLWDTVTGVDMAL* |
| 63 | Cleavage site | SLVPRGSPSR |
| 64 | Globular head | ICPKPAEYRNWSKPQCGITGFAPFSKDNSIRLSAGGDIW |
| | domain of NA of | VTREPYVSCDPDKCYQFALGQGTTINNVHSNNTARDR |
| | influenza virus | TPHRTLLMNELGVPFHLGTKQVCIAWSSSSCHDGKAW |
| | A/Kansas/14/2017 | LHVCITGDDKNATASFIYNGRLVDSVVSWSKDILRTQE |
| | | SECVCINGTCTVVMTDGNATGKADTKILFIEEGKIVHTS |
| | | KLSGSAQHVEECSCYPRYPGVRCVCRDNWKGSNRPIV |
| | | DINIKDHSIVSSYVCSGLVGDTPRKTDSSSSSHCLNPNN |
| | | EKGGHGVKGWAFDDGNDVWMGRTINETSRLGYETFK |
| | | VVEGWSNPKSKLQINRQVIVDRGDRSGYSGIFSVEGKS |
| | | CINRCFYVELIRGRKEETEVLWTSNSIVVFCGTSGTYGT |
| | | GSWPDGADLNLMHI |
| 65 | Globular head | LLLPEPEWTYPRLSCPGSTFQKALLISPHRFGETKGNSA |
| | domain of NA of | PLIIREPFVACGPNECKHFALTHYAAQPGGYYNGTRGD |
| | influenza virus | RNKLRHLISVKLGKIPTVENSIFHMAAWSGSACHDGKE |
| | B/Colorado/06/2017 | WTYIGVDGPDNNALLKVKYGEAYTDTYHSYANNILRT |
| | | QESACNCIGGNCYLMITDGSASGVSECRFLKIREGRIIK |
| | | EIFPTGRVKHTEECTCGFASNKTIECACRDNRYTAKRPF |
| | | VKLNVETDTAEIRLMCTDTYLDTPRPNDGSITGPCESD |
| | | GDKGSGGIKGGFVHQRMKSKIGRWYSRTMSQTERMG |
| | | MGLYVKYGGDPWADSDALAFSGVMVSMKEPGWYSF |
| | | GFEIKDKKCDVPCIGIEMVHDGGKETWHSAATAIYCL |
| | | MGSGQLLWDTVTGVDMAL* |
| 66 | Globular head | SVKLAGNSSLCPVSGWAIYSKDNSVRIGSKGDVFVIRE |
| | domain of NA of | PFISCSPLECRTFFLTQGALLNDKHSNGTIKDRSPYRTL |
| | influenza virus | MSCPIGEVPSPYNSRFESVAWSASACHDGINWLTIGISG |
| | A/Michigan/45/15 | PDSGAVAVLKYNGIITDTIKSWRNNILRTQESECACVN |
| | | GSCFTIMTDGPSDGQASYKIFRIEKGKIIKSVEMKAPNY |
| | | HYEECSCYPDSSEITCVCRDNWHGSNRPWVSFNQNLE |
| | | YQMGYICSGVFGDNPRPNDKTGSCGPVSSNGANGVKG |
| | | FSFKYGNGVWIGRTKSISSRKGFEMIWDPNGWTGTDN |
| | | KFSIKQDIVGINEWSGYSGSFVQHPELTGLDCIRPCFWV |
| | | ELIRGRPEENTIWTSGSSISFCGVNS |
| | | DTVGWSWPDGAELPFTIDK* |

### 5. DETAILED DESCRIPTION

In one aspect, provided herein are recombinant neuraminidases which form stable tetramers and are immunogenic. The recombinant neuraminidases may be used to immunize a subject against influenza virus.

### 5.1 Recombinant Influenza Virus Neuraminidase

Provided herein are recombinant influenza virus neuraminidases (NA), which may be used as immunogens. In a specific embodiment, a recombinant neuraminidase has the amino acid sequence of a neuraminidase described in Section 6.1 or 6.2, infra. In another specific embodiment, a recombinant neuraminidase has the amino acid sequence of a neuraminidase described in Section 6.1, infra, without one, two or more of the following: the signal sequence, histidine tag, or cleavage site.

In certain embodiments, a recombinant neuraminidase described herein retains one, two, or more, or all of the functions of a wild-type influenza virus neuraminidase. In a specific embodiment, a recombinant neuraminidase described herein cleaves sialic acid. Assays known to one skilled in the art can be utilized to assess the ability of a recombinant neuraminidase to cleave sialic acid.

It will be understood by those of skill in the art that a recombinant neuraminidase provided herein can be prepared according to any technique known by and deemed suitable to those of skill in the art, including the techniques described herein. In certain embodiments a recombinant neuraminidase described herein is isolated.

### 5.1.1 Recombinant Influenza Virus Neuraminidase with Mutated Ectodomains

In specific aspects, provided herein are recombinant neuraminidases comprising an influenza neuraminidase ectodomain with one, two or more amino acid substitutions to cysteine at one, two or more amino acid residues in the stalk domain of the ectodomain. In one embodiment, provided herein is a recombinant neuraminidase comprising an influenza virus neuraminidase globular head domain and stalk domain with one, two or more amino acid substitutions to cysteine at one, two or more amino acid residues in the stalk domain. In another embodiment, provided herein is a recombinant neuraminidase comprising an influenza virus neuraminidase globular head domain and stalk domain with one, two or more amino acid substitutions to cysteine at one, two or more amino acid residues in the region between the first cysteine of the globular head domain of the influenza virus neuraminidase and the transmembrane of the influenza virus neuraminidase. Those amino acid residues may include one, two or more of amino acid residues 48, 50, 61, 76, 78, and 81 of an N1 subtype or amino acid residues corresponding to one, two or more of amino acid residues 48, 50, 61, 76, 78, and 81 of influenza virus A/Puerto Rico/8/1934 neuraminidase. Alternatively, those amino acid residues may include one or both of amino acid residues 52 and 54 of an N2 subtype or amino acid residues corresponding to one or both of amino acid residues 52 and 54 of influenza virus A/Hong Kong/5738/2014. Without being bound by any theory, one or more amino acid substitutions resulting in one or more additional cysteines in the stalk domain of influenza virus neuraminidase result in the formation of disulfide bonds that increase the stability of the protein.

Those of skill in the art will recognize that the delineation of the domains of an influenza virus neuraminidase may be determined from, e.g., crystal structure and/or by using structure prediction software (for example, the website for the Center for Biological Sequence Analysis, Technical University of Denmark DTU, or Pymol) in conjunction with protein alignments. In a specific embodiment, the first cysteine of the globular head domain of influenza virus neuraminidase corresponds to the amino acid residue indicated by the bold and underlined asterisk in FIGS. 9A-9D.

The amino acid residues in the stalk domain of an influenza virus NA corresponding to particular amino acid residues (e.g., amino acid residues 48, 50, 61, 76, 78, and/or 81 of influenza virus A/Puerto Rico/8/1934 neuraminidase, or amino acid residues 52 and/or 54 of influenza virus A/Hong Kong//4801/2014) in another influenza virus NA may be identified using techniques known to one skilled in the art. In specific embodiments, the amino acid residues in the stalk domain of an influenza virus NA corresponding particular amino acid residues (e.g., amino acid residues 48, 50, 61, 76, 78, and/or 81 of influenza virus A/Puerto Rico/8/1934 neuraminidase, or amino acid residues 52 and/or 54 of influenza virus A/Hong Kong//4801/2014) are identified by comparing the amino acid sequences and/or structural information (e.g., crystal structures) of influenza virus NAs. In particular embodiments, alignments of the amino acid sequences of NA of influenza viruses (e.g., using ClustalOmega) as well as assessing the NAs for structural similarity enables the skilled person in the art to select the amino acid residues in the influenza virus NA that correspond to particular amino acid residues in stalk domain (e.g., amino acid residues 48, 50, 61, 76, 78, and/or 81 of influenza virus A/Puerto Rico/8/1934 neuraminidase, or amino acid residues 52 and/or 54 of influenza virus A/Hong Kong//4801/2014). See, e.g., the sequence alignments in FIGS. 9A-9D. In certain embodiments, one might want to refrain from substituting amino acid residues in the influenza virus NA stalk domain that impact the structure of the NA with cysteine since such an amino acid substitution may impact the folding of the NA. In other embodiments, any amino acid residue in the stalk domain of an influenza virus neuraminidase may be substituted with cysteine.

In one aspect, provided herein is a recombinant neuraminidase comprising a mutated influenza virus neuraminidase ectodomain, wherein the mutated ectodomain comprises an amino acid substitution to cysteine at amino acid residue 48, 50, 61, 76, 78, or 81 of an N1 subtype or at an amino acid residue corresponding to amino acid residue 48, 50, 61, 76, 78, or 81 of influenza virus A/Puerto Rico/8/1934 neuraminidase. In one embodiment, provided herein is a recombinant neuraminidase comprising a mutated influenza virus neuraminidase ectodomain, wherein the mutated ectodomain comprises an amino acid substitution to cysteine at amino acid residue 48 of an N1 subtype or at an amino acid residue corresponding to amino acid residue 48 of influenza virus A/Puerto Rico/8/1934 neuraminidase. In another embodiment, provided herein is a recombinant neuraminidase comprising a mutated influenza virus neuraminidase ectodomain, wherein the mutated ectodomain comprises an amino acid substitution to cysteine at amino acid residue 50 of an N1 subtype or at an amino acid residue corresponding to amino acid residue 50 of influenza virus A/Puerto Rico/8/1934 neuraminidase. In another embodiment, provided herein is a recombinant neuraminidase comprising a mutated influenza virus neuraminidase ectodomain, wherein the mutated ectodomain comprises an amino acid substitution to cysteine at amino acid residue 61 of an N1 subtype or at an amino acid residue corresponding to amino acid residue 61 of influenza virus A/Puerto Rico/8/1934 neuraminidase. In another embodiment, provided herein is a recombinant neuraminidase comprising a mutated influenza virus neuraminidase ectodomain, wherein the mutated ectodomain comprises an amino acid substitution to cysteine at amino acid residue 76 of an N1 subtype or at an amino acid residue corresponding to amino acid residue 76 of influenza virus A/Puerto Rico/8/1934 neuraminidase. In another embodiment, provided herein is a recombinant neuraminidase comprising a mutated influenza virus neuraminidase ectodomain, wherein the mutated ectodomain comprises an amino acid substitution to cysteine at amino acid residue 81 of an N1 subtype or at an amino acid residue corresponding to amino acid residue 81 of influenza virus A/Puerto Rico/8/1934 neuraminidase. In specific embodiments, the mutated ectodomain, aside from the amino acid residue substitutions described herein, comprises the amino acid residues corresponding to the wild-type influenza virus ectodomain.

In another aspect, provided herein is a recombinant neuraminidase comprising a mutated influenza virus neuraminidase ectodomain, wherein the mutated ectodomain comprises amino acid substitutions to cysteine at two, three, or more of amino acid residues 48, 50, 61, 76, 78, and 81 of an N1 subtype or at amino acid residues corresponding to two, three or more of amino acid residues 48, 50, 61, 76, 78, and 81 of influenza virus A/Puerto Rico/8/1934 neuraminidase. In a specific embodiment, provided herein is a recombinant influenza virus comprising a mutated influenza virus neuraminidase ectodomain, wherein the mutated ectodomain comprises amino acid substitutions to cysteine at amino acid residues 48 and 50 of an N1 subtype or amino acid residues corresponding to amino acid residues 48 and 50 of influenza virus A/Puerto Rico/8/1934 neuraminidase. In specific embodiments, the mutated ectodomain, aside from the amino acid residue substitutions described herein, comprises the amino acid residues corresponding to the wild-type influenza virus neuraminidase ectodomain.

In another aspect, provided herein is a recombinant neuraminidase comprising a mutated influenza virus neuraminidase ectodomain, wherein the mutated ectodomain comprises an amino acid substitution to cysteine at amino acid residue 48, 50, 61, 76, 78, or 81 of influenza virus A/Michigan/45/2015 neuraminidase or at an amino acid residue corresponding to amino acid residue 48, 50, 61, 76, 78, or 81 of influenza virus A/Michigan/45/2015 neuraminidase. In one embodiment, provided herein is a recombinant neuraminidase comprising a mutated influenza virus neuraminidase ectodomain, wherein the mutated ectodomain comprises an amino acid substitution to cysteine at amino acid residue 48 of influenza virus A/Michigan/45/2015 neuraminidase or at an amino acid residue corresponding to amino acid residue 48 of influenza virus A/Michigan/45/2015 neuraminidase. In another embodiment, provided herein is a recombinant neuraminidase comprising a mutated influenza virus neuraminidase ectodomain, wherein the mutated ectodomain comprises an amino acid substitution to cysteine at amino acid residue 50 of influenza virus A/Michigan/45/2015 neuraminidase or at an amino acid residue corresponding to amino acid residue 50 of influenza virus A/Michigan/45/2015 neuraminidase. In another embodiment, provided herein is a recombinant neuraminidase comprising a mutated influenza virus neuraminidase ectodomain, wherein the mutated ectodomain comprises an amino acid substitution to cysteine at amino acid residue 61 of influenza virus A/Michigan/45/2015 neuraminidase or at an amino acid residue corresponding to amino acid residue 61 of influenza virus A/Michigan/45/2015 neuraminidase. In another embodiment, provided herein is a recombinant neuraminidase comprising a mutated influenza virus neuraminidase ectodomain, wherein the mutated ectodomain comprises an amino acid substitution to cysteine at amino acid residue 76 of influenza virus A/Michigan/45/2015 neuraminidase or at an amino acid residue corresponding to amino acid residue 76 of influenza virus A/Michigan/45/2015 neuraminidase. In another embodiment, provided herein is a recombinant neuraminidase comprising a mutated influenza virus neuraminidase ectodomain, wherein the mutated ectodomain comprises an amino acid substitution to cysteine at amino acid residue 81 of influenza virus A/Michigan/45/2015 neuraminidase or at an amino acid residue corresponding to amino acid residue 81 of influenza virus A/Michigan/45/2015 neuraminidase. In specific embodiments, the mutated ectodomain, aside from the amino acid residue substitutions described herein, comprises the amino acid residues corresponding to the wild-type influenza virus A/Michigan/45/2015 neuraminidase ectodomain.

In another aspect, provided herein is a recombinant neuraminidase comprising a mutated influenza A virus neuraminidase ectodomain, wherein the mutated ectodomain comprises an amino acid substitution to cysteine at amino acid residue 52 or 54 of an N2 subtype or at an amino acid residue corresponding to amino acid residue 52 or 54 of influenza virus A/Hong Kong/5738/2014. In a specific embodiment, provided herein is a recombinant neuraminidase comprising a mutated ectodomain, wherein the mutated ectodomain comprises amino acid substitutions at one, two or more of amino acid residues of the influenza virus A/Hong Kong/5738/2014 neuraminidase that correspond to amino acid residues 61, 76, 78 and/or 81 of A/Puerto Rico/8/1934 neuraminidase. In a specific embodiment, provided herein is a recombinant neuraminidase comprising a mutated influenza A virus neuraminidase ectodomain, wherein the mutated ectodomain comprises an amino acid substitution to cysteine at amino acid residue 52 of an N2 subtype or at an amino acid residue corresponding to amino acid residue 52 of influenza virus A/Hong Kong/5738/2014. In another specific embodiment, provided herein is a recombinant neuraminidase comprising a mutated influenza A virus neuraminidase ectodomain, wherein the mutated ectodomain comprises an amino acid substitution to cysteine at amino acid residue 54 of an N2 subtype or at an amino acid residue corresponding to amino acid residue 54 of influenza virus A/Hong Kong/5738/2014. In another specific embodiment, provided herein is a recombinant neuraminidase comprising a mutated influenza A virus neuraminidase ectodomain, wherein the mutated ectodomain comprises amino acid substitutions to cysteine at amino acid residues 52 and 54 of an N2 subtype or at amino acid residues corresponding to amino acid residues 52 and 54 of influenza virus A/Hong Kong/5738/2014. In specific embodiments, the mutated ectodomain, aside from the amino acid residue substitutions described herein, comprises the amino acid residues corresponding to the wild-type influenza virus neuraminidase ectodomain.

In another aspect, provided herein are recombinant neuraminidases comprising an influenza neuraminidase ectodomain with one, two or more insertions of a cysteine at one, two or more amino acid residues in the stalk domain of the ectodomain. In one embodiment, provided herein is a recombinant neuraminidase comprising an influenza virus neuraminidase globular head domain and stalk domain with one, two or more insertions of a cysteine at one, two or more amino acid residues in the stalk domain. In another embodiment, provided herein is a recombinant neuraminidase comprising an influenza virus neuraminidase globular head domain and stalk domain with one, two or more amino acid substitutions to cysteine at one, two or more amino acid residues in the region between the first cysteine of the globular head domain of the influenza virus neuraminidase and the transmembrane of the influenza virus neuraminidase. Those insertions may occur at amino acid residues 48, 50, 61, 76, 78, and 81 of an N1 subtype or at amino acid residues corresponding to one, two or more of amino acid residues 48, 50, 61, 76, 78, and 81 of influenza virus A/Puerto Rico/8/1934 neuraminidase. In specific embodiments, the mutated ectodomain, aside from the insertion of one or more cysteines described herein, comprises the amino acid residues corresponding to the wild-type influenza virus neuraminidase ectodomain.

In one embodiment, provided herein is a recombinant neuraminidase comprising a mutated influenza virus neuraminidase ectodomain, wherein the mutated ectodomain comprises an insertion of a cysteine at amino acid residue 48, 50, 61, 76, 78, or 81 of an N1 subtype or at an amino acid residue corresponding to amino acid residue 48, 50, 61, 76, 78, or 81 of influenza virus A/Puerto Rico/8/1934 neuraminidase. In one embodiment, provided herein is a recombinant neuraminidase comprising a mutated influenza virus neuraminidase ectodomain, wherein the mutated ectodomain comprises an insertion of a cysteine at amino acid residue 61 of an N1 subtype or at an amino acid residue corresponding to amino acid residue 61 of influenza virus A/Puerto Rico/8/1934 neuraminidase. In specific embodiments, the mutated ectodomain, aside from the insertion of one or more cysteines described herein, comprises the amino acid residues corresponding to the wild-type influenza virus neuraminidase ectodomain.

The location to insert cysteine in the stalk domain of an influenza virus NA may be identified using techniques known to one skilled in the art. In specific embodiments, the location to insert cysteine in the stalk domain of an influenza virus NA are identified by comparing the amino acid sequences and/or structural information (e.g., crystal structures) of influenza virus NAs. In particular embodiments, alignments of the amino acid sequences of NA of influenza viruses (e.g., using ClustalOmega) as well as assessing the NAs for structural similarity enables the skilled person in the art to select the location to insert cysteine.

In another aspect, provided herein are recombinant neuraminidases comprising an influenza neuraminidase ectodomain with one, two or more insertions of a cysteine at particular amino acid residues in the stalk domain of the ectodomain and one, two or more amino acid substitutions to cysteine at particular amino acid residues in the stalk domain of the ectodomain. In one embodiment, provided herein is a recombinant neuraminidase comprising an influenza virus neuraminidase globular head domain and stalk domain with one, two or more insertions of a cysteine at particular amino acid residues in the stalk domain and one, two or more amino acid substitutions to cysteine at particular amino acid residues in the stalk domain of ectodomain. Those insertions, amino acid substitutions or both may occur at amino acid residues 48, 50, 61, 76, 78, and 81 of an N1 subtype or at amino acid residues corresponding to one, two or more of amino acid residues 48, 50, 61, 76, 78, and 81 of influenza virus A/Puerto Rico/8/1934 neuraminidase. In one embodiment, provided herein is a recombinant neuraminidase comprising a mutated influenza virus neuraminidase ectodomain, wherein the mutated ectodomain comprises (1) an insertion of a cysteine at amino acid residue 61 of an N1 subtype or at an amino acid residue corresponding to amino acid residue 61 of influenza virus A/Puerto Rico/8/1934 neuraminidase; and (2) an amino acid substitution to cysteine at amino acid residue 48 of an N1 subtype or at an amino acid residue corresponding to amino acid residue 48 of influenza virus A/Puerto Rico/8/1934 neuraminidase. In specific embodiments, the mutated ectodomain, aside from the insertion of one or more cysteines described herein and the amino acid residue substitutions described herein, comprises the amino acid residues corresponding to the wild-type influenza virus ectodomain.

In specific embodiments, a mutated influenza virus ectodomain is based on, from or derived from an influenza A virus neuraminidase ectodomain, such as, e.g., an N1, N2, N3, N4, N5, N6, N7, N8 or N9 subtype ectodomain. In some embodiments, a mutated influenza virus ectodomain is based on, from or derived from N1, N2, N3, N4, N5, N6, N7, N8, N9, N10 or N11 ectodomain. In certain embodiments, a mutated influenza virus ectodomain is based on, from or derived from an influenza A virus neuraminidase ectodomain that has 70%, 75%, 80%, or 85% identity to the ectodomain of an influenza A virus neuraminidase described herein or known in the art. In some embodiments, a mutated influenza virus ectodomain is based on, from or derived from an influenza A virus neuraminidase ectodomain that has 90%, 95%, or 98% identity to the ectodomain of an influenza A virus neuraminidase described herein or known in the art. In other embodiments, a mutated influenza virus ectodomain is based on, from or derived from an influenza B virus neuraminidase ectodomain. In certain embodiments, a mutated influenza virus ectodomain is based on, from or derived from an influenza B virus neuraminidase ectodomain that has 70%, 75%, 80%, or 85% identity to the ectodomain of an influenza B virus neuraminidase described herein or known in the art. In some embodiments, a mutated influenza virus ectodomain is based on, from or derived from an influenza B virus neuraminidase ectodomain that has 90%, 95%, or 98% identity to the ectodomain of an influenza B virus neuraminidase described herein or known in the art. Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length. In a certain embodiment, the percent identity is determined over the entire length of an amino acid sequence or nucleotide sequence. The determination of percent identity between two sequences (e.g., amino acid sequences or nucleic acid sequences) can also be accomplished using a mathematical algorithm. A non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). In another non-limiting example of a mathematical algorithm utilized for the comparison of sequences is the algorithm of Myers and Miller, 1988, CABIOS 4: 11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, a recombinant neuraminidase provided herein further comprises an influenza virus neuraminidase transmembrane domain, cytoplasmic domain or both. In a specific embodiment, a recombinant neuraminidase provided herein comprises a mutated influenza virus neuraminidase ectodomain described herein and an influenza virus neuraminidase transmembrane domain, cytoplasmic domain or both. In certain embodiments, the transmembrane domain, cytoplasmic domain or both are based on, from or derived from an influenza A virus neuraminidase, such as, e.g., an N1, N2, N3, N4, N5, N6, N7, N8 or N9 subtype. In some embodiments, the transmembrane domain, cytoplasmic domain or both are based on, from or derived from N1, N2, N3, N4, N5, N6, N7, N8, N9, N10 or N11. In certain embodiments, the transmembrane domain, cytoplasmic domain or both are based on, from or derived from the transmembrane domain, cytoplasmic domain or both of an influenza A virus neuraminidase that has 70%, 75%, 80%, or 85% identity to the transmembrane domain, cytoplasmic domain or both of an influenza A virus neuraminidase described herein or known in the art. In some embodiments, the transmembrane domain, cytoplasmic domain or both are based on, from or derived from the transmembrane domain, cytoplasmic domain or both of an influenza A virus neuraminidase that has 90%, 95%, or 98% identity to the transmembrane domain, cytoplasmic domain or both of an influenza A virus neuraminidase described herein or known in the art. In other embodiments, the transmembrane domain, cytoplasmic domain or both based on, from or derived from an influenza B virus neuraminidase. In some embodiments, the transmembrane domain, cytoplasmic domain or both are based on, from or derived from the transmembrane domain, cytoplasmic domain or both of an influenza B virus neuraminidase that has 70%, 75%, 80%, or 85% identity to the transmembrane domain, cytoplasmic domain or both of an influenza B virus neuraminidase described herein or known in the art. In certain embodiments, the transmembrane domain, cytoplasmic domain or both are based on, from or derived from the transmembrane domain, cytoplasmic domain or both of an influenza B virus neuraminidase that has 70%, 75%, 80%, or 85% identity to the transmembrane domain, cytoplasmic domain or both of an influenza B virus neuraminidase described herein or known in the art. In a specific embodiment, a recombinant neuraminidase provided herein further comprises an influenza virus neuraminidase transmembrane domain, cytoplasmic domain or both, which is based on, from or derived from the same influenza virus neuraminidase as the mutated ectodomain. In another embodiment, a recombinant neuraminidase provided herein further comprises an influenza virus neuraminidase transmembrane domain, cytoplasmic domain or both, which is based on, from or derived from the same influenza virus neuraminidase subtype as the mutated ectodomain.

In certain embodiments, a recombinant neuraminidase provided herein is a soluble. In other embodiments, a recombinant neuraminidase provided herein is not soluble.

In certain embodiments, a recombinant neuraminidase provided herein comprises a tetramerization domain. In a specific embodiment, a recombinant neuraminidase provided herein comprises a mutated influenza virus neuraminidase ectodomain described herein and a tetramerization domain. In some embodiments, the tetramerization domain comprises a tetramerization domain from a GCN4 leucine zipper, a bacterial tetrabrachion tetramerization domain or the human vasodilator stimulated phosphoprotein (VASP) tetramerization domain. In certain embodiments, the tetramerization domain comprises the tetramerization domain from SEPPALLATA-like MADS domain transcription factor from Arabidopsis thaliana (SMDTF), PiLZ structure from Xhantomonas campestris, or Dictyocaulus viviparus ACE tetramerization domain. In other embodiments, the tetramerization domain comprises a tetramerization domain from a paramyxovirus phosphoprotein (e.g., a Nipah virus phosphoprotein, a Hendra virus phosphoprotein, a respiratory syncytial virus phosphoprotein, human parainfluenza virus (hPIV) phosphoprotein, bovine parainfluenza virus phosphoprotein, a mumps virus phosphoprotein, a Cedar virus phosphoprotein, a Ghana virus phosphoprotein, a Newcastle disease virus phosphoprotein, a canine distemper virus phosphoprotein, or a Peste des petits ruminants virus (PPRV) phosphoprotein). In a specific embodiment, the tetramerization domain is a tetramerization from a measles virus phosphoprotein tetramerization domain or a Sendai virus phosphoprotein tetramerization domain. In another specific embodiment, the tetramerization domain comprises the amino acid sequence of SEQ ID NO: 1, 2, 3, 4 or 5.

In certain embodiments, the tetramerization used has been modified. For example, a tetramerization domain, such as those noted above, has been immunologically silenced by, e.g., adding N-linked glycans to epitopes (e.g., immunodominant epitopes). The epitopes (e.g., immunodominant epitopes) of a tetramerization domain, such as those noted above, may be dampened by adding N-linked glycosylation sites (e.g., Asn-Xaa-Ser/Thr, wherein Xaa is any amino acid, or Asn-Xaa-Ser/Thr, wherein Xaa is any amino acid except Pro) to the domain. In certain embodiments, one, two or more non-naturally occurring glycosylation sites are introduced into the tetramerization domain. The locations of the non-naturally occurring glycosylation sites may be chosen so they cover the immunodominant epitopes of the tetramerization domain. The presence of N-linked glycans may make the covered epitopes immunologically inert.

In certain embodiments, provided herein is a recombinant neuraminidase comprising a mutated influenza virus ectodomain which comprises an influenza A virus neuraminidase ectodomain, such as, e.g., an N1, N2, N3, N4, N5, N6, N7, N8 or N9 subtype ectodomain, with an amino acid substitution to cysteine at amino acid residue 48, 50, 61, 76, 78, or 81 of an N1 subtype or at an amino acid residue corresponding to amino acid residue 48, 50, 61, 76, 78, or 81 of influenza virus A/Puerto Rico/8/1934 neuraminidase. In other embodiments, provided herein is a recombinant neuraminidase comprising a mutated influenza virus ectodomain which comprises an influenza A virus neuraminidase ectodomain, such as, e.g., an N1, N2, N3, N4, N5, N6, N7, N8 or N9 subtype ectodomain, with amino acid substitutions to cysteine at two, three or more of amino acid residues 48, 50, 61, 76, 78, and 81 of an N1 subtype or at amino acid residues corresponding to two, three or more of amino acid residues 48, 50, 61, 76, 78, and 81 of influenza virus A/Puerto Rico/8/1934 neuraminidase. In certain embodiments, provided herein is a recombinant neuraminidase comprising a mutated influenza virus ectodomain which comprises an influenza A virus neuraminidase ectodomain, such as, e.g., an N1, N2, N3, N4, N5, N6, N7, N8 or N9 subtype ectodomain, with amino acid substitutions to cysteine at amino acid residues 48 and 50 of an N1 subtype or at amino acid residues corresponding to amino acid residues 48 and 50 of influenza virus A/Puerto Rico/8/1934 neuraminidase. In some embodiments, the recombinant neuraminidase further comprises a transmembrane domain, a cytoplasmic domain or both from an influenza virus NA subtype that is the same as the NA subtype of the influenza virus ectodomain. In certain embodiments, the recombinant neuraminidase further comprises a transmembrane domain, a cytoplasmic domain or both from the same influenza A virus neuraminidase as the ectodomain. In other embodiments, the recombinant neuraminidase further comprises a tetramerization domain, such as described herein.

In certain embodiments, provided herein is a recombinant neuraminidase comprising a mutated influenza virus ectodomain which comprises an influenza B virus neuraminidase ectodomain with an amino acid substitution to cysteine at an amino acid residue corresponding to amino acid residue 48, 50, 61, 76, 78, or 81 of influenza virus A/Puerto Rico/8/1934 neuraminidase. In a specific embodiment, provided herein is a recombinant neuraminidase comprising a mutated influenza virus ectodomain which comprises an influenza B virus neuraminidase ectodomain with amino acid substitutions to cysteine at amino acid residues corresponding to two, three or more of amino acid residues 48, 50, 61, 76, 78, and 81 of influenza virus A/Puerto Rico/8/1934 neuraminidase. In some embodiments, the recombinant neuraminidase further comprises a transmembrane domain, a cytoplasmic domain or both from an influenza virus NA linage that is the same as the NA lineage of the influenza virus ectodomain. In certain embodiments, the recombinant neuraminidase further comprises a transmembrane domain, a cytoplasmic domain or both from the same influenza B virus neuraminidase as the ectodomain. In other embodiments, the recombinant neuraminidase further comprises a tetramerization domain, such as described herein.

In certain embodiments, provided herein is a recombinant neuraminidase comprising a mutated influenza A virus neuraminidase ectodomain which comprises an influenza A virus neuraminidase ectodomain, such as, e.g., an N1, N2, N3, N4, N5, N6, N7, N8 or N9 subtype ectodomain, with an amino acid substitution to cysteine at an amino acid residue corresponding to amino acid residue 52 or 54 of influenza virus A/Hong Kong/5738/2014 neuraminidase. In some embodiments, provided herein is a recombinant neuraminidase comprising a mutated influenza A virus neuraminidase ectodomain which comprises an influenza A virus neuraminidase ectodomain, such as, e.g., an N1, N2, N3, N4, N5, N6, N7, N8 or N9 subtype ectodomain, with amino acid substitutions to cysteine at amino acid residues corresponding to amino acid residues 52 and 54 of influenza virus A/Hong Kong/5738/2014 neuraminidase. In some embodiments, the recombinant neuraminidase further comprises a transmembrane domain, a cytoplasmic domain or both from an influenza virus NA subtype that is the same as the NA subtype of the influenza virus ectodomain. In certain embodiments, the recombinant neuraminidase further comprises a transmembrane domain, a cytoplasmic domain or both from the same influenza A virus neuraminidase as the ectodomain. In other embodiments, the recombinant neuraminidase further comprises a tetramerization domain, such as described herein.

In certain embodiments, provided herein is a recombinant neuraminidase comprising a mutated influenza B virus neuraminidase ectodomain which comprises an influenza B virus neuraminidase ectodomain with an amino acid substitution to cysteine at an amino acid residue corresponding to amino acid residue 52 or 54 of influenza virus A/Hong Kong/5738/2014 neuraminidase. In some embodiments, provided herein is a recombinant neuraminidase comprising a mutated influenza B virus neuraminidase ectodomain which comprises an influenza B virus neuraminidase ectodomain with amino acid substitutions to cysteine at amino acid residues corresponding to amino acid residues 52 and 54 of influenza virus A/Hong Kong/5738/2014 neuraminidase. In some embodiments, the recombinant neuraminidase further comprises a transmembrane domain, a cytoplasmic domain or both from an influenza virus NA lineage that is the same as the NA lineage of the influenza virus ectodomain. In certain embodiments, the recombinant neuraminidase further comprises a transmembrane domain, a cytoplasmic domain or both from the same influenza B virus neuraminidase as the ectodomain. In other embodiments, the recombinant neuraminidase further comprises a tetramerization domain, such as described herein.

In certain embodiments, provided herein is a recombinant neuraminidase comprising a mutated influenza virus ectodomain which comprises an influenza A virus neuraminidase ectodomain, such as, e.g., an N1, N2, N3, N4, N5, N6, N7, N8 or N9 subtype ectodomain, with an insertion of a cysteine at amino acid residue 48, 50, 61, 76, 78, or 81 of an N1 subtype or at an amino acid residue corresponding to amino acid residue 48, 50, 61, 76, 78, or 81 of influenza virus A/Puerto Rico/8/1934 neuraminidase. In other embodiments, provided herein is a recombinant neuraminidase comprising a mutated influenza virus ectodomain which comprises an influenza A virus neuraminidase ectodomain, such as, e.g., an N1, N2, N3, N4, N5, N6, N7, N8 or N9 subtype ectodomain, with an insertion of a cysteine at two, three or more of amino acid residues 48, 50, 61, 76, 78, and 81 of an N1 subtype or at amino acid residues corresponding to two, three or more of amino acid residues 48, 50, 61, 76, 78, and 81 of influenza virus A/Puerto Rico/8/1934 neuraminidase. In some embodiments, the recombinant neuraminidase further comprises a transmembrane domain, a cytoplasmic domain or both from an influenza virus NA subtype that is the same as the NA subtype of the influenza virus ectodomain. In certain embodiments, the recombinant neuraminidase further comprises a transmembrane domain, a cytoplasmic domain or both from the same influenza A virus neuraminidase as the ectodomain. In other embodiments, the recombinant neuraminidase further comprises a tetramerization domain, such as described herein.

In certain embodiments, provided herein is a recombinant neuraminidase comprising a mutated influenza virus ectodomain which comprises an influenza B virus neuraminidase ectodomain with an insertion of a cysteine at an amino acid residue corresponding to amino acid residue 48, 50, 61, 76, 78, or 81 of influenza virus A/Puerto Rico/8/1934 neuraminidase. In other embodiments, provided herein is a recombinant neuraminidase comprising a mutated influenza virus ectodomain which comprises an influenza B virus neuraminidase ectodomain with an insertion of a cysteine at amino acid residues corresponding to two, three or more of amino acid residues 48, 50, 61, 76, 78, and 81 of influenza virus A/Puerto Rico/8/1934 neuraminidase. In some embodiments, the recombinant neuraminidase further comprises a transmembrane domain, a cytoplasmic domain or both from an influenza virus NA lineage that is the same as the NA lineage of the influenza virus ectodomain. In certain embodiments, the recombinant neuraminidase further comprises a transmembrane domain, a cytoplasmic domain or both from the same influenza B virus neuraminidase as the ectodomain. In other embodiments, the recombinant neuraminidase further comprises a tetramerization domain, such as described herein.

In certain embodiments, provided herein is a recombinant neuraminidase comprising a mutated influenza virus ectodomain which comprises an influenza A virus neuraminidase ectodomain, such as, e.g., an N1, N2, N3, N4, N5, N6, N7, N8 or N9 subtype ectodomain, with (1) an insertion of a cysteine at amino acid residue 48, 50, 61, 76, 78, or 81 of an N1 subtype or at an amino acid residue corresponding to amino acid residue 48, 50, 61, 76, 78, or 81 of influenza virus A/Puerto Rico/8/1934 neuraminidase, and (2) an amino acid substitution to cysteine at amino acid residue 48, 50, 61, 76, 78, or 81 of an N1 subtype or at an amino acid residue corresponding to amino acid residue 48, 50, 61, 76, 78, or 81 of influenza virus A/Puerto Rico/8/1934 neuraminidase. In other embodiments, provided herein is a recombinant neuraminidase comprising a mutated influenza virus ectodomain which comprises an influenza A virus neuraminidase ectodomain, such as, e.g., an N1, N2, N3, N4, N5, N6, N7, N8 or N9 subtype ectodomain, with (1) an insertion of a cysteine at one, two, three or more of amino acid residues 48, 50, 61, 76, 78, and 81 of an N1 subtype or at amino acid residues corresponding to one, two, three or more of amino acid residues 48, 50, 61, 76, 78, and 81 of influenza virus A/Puerto Rico/8/1934 neuraminidase; and (2) an amino acid substitution to cysteine at one, two, three or more of amino acid residues 48, 50, 61, 76, 78, and 81 of an N1 subtype or at amino acid residues corresponding to one, two, three or more of amino acid residues 48, 50, 61, 76, 78, and 81 of influenza virus A/Puerto Rico/8/1934 neuraminidase. In a specific embodiment, provided herein is a recombinant neuraminidase comprising a mutated influenza virus ectodomain which comprises an influenza A virus neuraminidase ectodomain, such as, e.g., an N1, N2, N3, N4, N5, N6, N7, N8 or N9 subtype ectodomain, with (1) an insertion of a cysteine at amino acid residue 61 of an N1 subtype or at an amino acid residue corresponding to amino acid residue 61 of influenza virus A/Puerto Rico/8/1934 neuraminidase, and (2) an amino acid substitution to cysteine at amino acid residue 48 of an N1 subtype or at an amino acid residue corresponding to amino acid residue 48 of influenza virus A/Puerto Rico/8/1934 neuraminidase. In some embodiments, the recombinant neuraminidase further comprises a transmembrane domain, a cytoplasmic domain or both from an influenza virus NA subtype that is the same as the NA subtype of the influenza virus ectodomain. In certain embodiments, the recombinant neuraminidase further comprises a transmembrane domain, a cytoplasmic domain or both from the same influenza A virus neuraminidase as the ectodomain. In other embodiments, the recombinant neuraminidase further comprises a tetramerization domain, such as described herein.

In certain embodiments, provided herein is a recombinant neuraminidase comprising a mutated influenza virus ectodomain which comprises an influenza B virus neuraminidase ectodomain with (1) an insertion of a cysteine at an amino acid residue corresponding to amino acid residue 48, 50, 61, 76, 78, or 81 of influenza virus A/Puerto Rico/8/1934neuraminidase, and (2) an amino acid substitution to cysteine at amino acid residue corresponding to amino acid residue 48, 50, 61, 76, 78, or 81 of influenza virus A/Puerto Rico/8/1934 neuraminidase. In other embodiments, provided herein is a recombinant neuraminidase comprising a mutated influenza virus ectodomain which comprises an influenza B virus neuraminidase ectodomain with (1) an insertion of a cysteine at amino acid residues corresponding to one, two, three or more of amino acid residues 48, 50, 61, 76, 78, and 81 of influenza virus A/Puerto Rico/8/1934 neuraminidase, and (2) an amino acid substitution to cysteine at one, two, three or more of amino acid residues corresponding to one, two, three or more of amino acid residues 48, 50, 61, 76, 78, and 81 of influenza virus A/Puerto Rico/8/1934 neuraminidase. In some embodiments, the recombinant neuraminidase further comprises a transmembrane domain, a cytoplasmic domain or both from an influenza virus NA lineage that is the same as the NA lineage of the influenza virus ectodomain. In certain embodiments, the recombinant neuraminidase further comprises a transmembrane domain, a cytoplasmic domain or both from the same influenza B virus neuraminidase as the ectodomain. In other embodiment, the recombinant neuraminidase further comprises a tetramerization domain, such as described herein.

In certain embodiments, an influenza virus neuraminidase is a human influenza virus neuraminidase. Human influenza virus neuraminidases are known in the art. In certain embodiments, an influenza virus neuraminidase is a swine influenza virus neuraminidase. Swine influenza virus neuraminidases are known in the art. In certain embodiments, an influenza virus neuraminidase is an equine influenza virus neuraminidase. Equine influenza virus neuraminidases are known in the art. In certain embodiments, an influenza virus neuraminidase is an avian influenza virus neuraminidase. Avian influenza virus neuraminidases are known in the art. In certain embodiments, an influenza virus neuraminidase is a seal influenza virus neuraminidase. Seal influenza virus neuraminidases are known in the art. Examples of specific influenza A virus neuraminidases include the neuraminidase of an influenza A virus strains described herein. In a specific embodiment, the influenza A virus neuraminidase is influenza A/Brisbane/02/2018 (H1N1)pdm09-like virus neuraminidase, influenza A/Kansas/14/2017 (H3N2)-like virus neuraminidase, influenze A/Brisbane/02/2018 (H1N1)pdm09-like virus neuraminidase, or influenza A/South Australia/34/2019 (H3N2)-like virus neuraminidase.

In certain embodiments, an influenza B virus neuraminidase is a human influenza B virus neuraminidase. Human influenza B virus neuraminidases are known in the art. In certain embodiments, an influenza B virus neuraminidase is a seal influenza B virus neuraminidase. Seal influenza B virus neuraminidases are known in the art. Examples of specific influenza B virus neuraminidases include the neuraminidase of an influenza B/Yamagata/16/88-lineage virus or an influenza virus B/Victoria/2/87-lineage virus. In a specific embodiment, the influenza B virus neuraminidase is influenza a B/Colorado/06/2017-like virus (B/Victoria/2/87 lineage) neuraminidase, influenza B/Phuket/3073/2013-like virus (B/Yamagata/16/88 lineage) neuraminidase, influenza B/Washington/02/2019-like (B/Victoria lineage) virus neuraminidase, or influenza B/Phuket/3073/2013-like (B/Yamagata lineage) virus neuraminidase.

GenBank™ Accession No. AAA43397.1 provides an exemplary amino acid sequence for a human influenza virus neuraminidase. GenBank™ Accession No. ABG23658.1 (GI: 108946273), GenBank™ Accession No. NP_040981.1 (GI: 8486128), GenBank™ Accession No. AAA43412.1 (GI: 324508), GenBank™ Accession No. ABE97720.1 (GI: 93008579), GenBank™ Accession No. ABE97719.1 (GI: 93008577), and GenBank™ Accession No. ABE97718.1 (GI: 93008575) provide exemplary amino acid sequences for human influenza virus neuraminidases. GenBank™ Accession No. CR106477.1 provides an exemplary amino acid sequence for a swine influenza virus neuraminidase. GenBank™ Accession No. AAQ90293.1 provides an exemplary amino acid sequence for an equine influenza virus neuraminidase. GenBank™ Accession No. AEX30531.1 (GI: 371449652), GenBank™ Accession No. AEX30532.1 (GI: 371449654), GenBank™ Accession No. AIA62041.1 (GI: 641454926), GenBank™ Accession No. AI130325.1 (GI: 670605039), GenBank™ Accession No. AGO18161.1 (GI: 513130855), and GenBank™ Accession No. AAS89005.1 (GI: 46360357) provide exemplary amino acid sequences for avian influenza virus neuraminidases. Sequences of influenza virus genes may also be found in the Influenza Research Database. For example, influenza virus neuraminidase sequences may be found in the Influenza Research Database under Accession No. FJ66084 and Accession No. KF90392.

In a specific embodiment, a recombinant neuraminidase provided herein has the amino acid sequence of SEQ ID NO: 7, 8, 9, 10, 11, 13 or 14. In another specific embodiment, a recombinant neuraminidase provided herein has the amino acid sequence of SEQ ID NO: 16, 17, 18, 19, 20, 21, 22, or 23. In another specific embodiment, a recombinant neuraminidase provided herein has the amino acid sequence of SEQ ID NO: 16, 17, 18, 19, 20, 21, 22, or 23 without one, two or all of the following: signal peptide, hexa-histidine tag and thrombin cleavage site.

In certain embodiments, a recombinant neuraminidase provided herein further comprise one or more polypeptide domains. Useful polypeptide domains include domains that facilitate purification, folding and cleavage of portions of a polypeptide. For example, a His tag (His-His-His-His-His-His) (SEQ ID NO:50), FLAG epitope or other purification tag can facilitate purification of an neuraminidase polypeptide provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. Cleavage sites can be used to facilitate cleavage of a portion of a polypeptide, for example cleavage of a purification tag. Useful cleavage sites include a thrombin cleavage site, for example one with the sequence LVPRGSP (SEQ ID NO:51). In a specific embodiment, the cleavage site comprises the amino acid sequence of SLVPRGSPSR (SEQ ID NO:63). In certain embodiments, the cleavage site is a cleavage site recognized by Tobacco Etch Virus (TEV) protease (e.g., amino acid sequence Glu-Asn-Leu-Tyr-Phe-Gln-(Gly/Ser) (SEQ ID NO:52)).

In certain embodiments, a recombinant neuraminidase provided herein exists in one, two, three or all of the following forms: monomeric, dimeric, trimeric or tetrameric. In specific embodiments, a recombinant neuraminidase provided herein is tetrameric as assessed by techniques known in the art or described herein.

In specific embodiments, a recombinant neuraminidase provided herein are capable of forming a three-dimensional structure that is similar to the three-dimensional structure of a native influenza neuraminidase. Structural similarity might be evaluated based on any technique deemed suitable by those of skill in the art. For instance, reaction, e.g. under non-denaturing conditions, of a recombinant neuraminidase with a neutralizing antibody or antiserum that recognizes a native influenza neuraminidase might indicate structural similarity. Useful neutralizing antibodies or antisera are described in, e.g., Shoji et al., Hum. Vaccines, 2011, 7:199-204, Wan et al., J. Virol. 2013, 87:9290-9300, Doyle et al. Antivir. Res. 2013, 100:567-574, Doyle et al., Biochem. Biophys. Res. Commun. 2013, 441:226-229, and Wohlbold et al., 2017, Nat.. Microbiol. 2(10): 1415-1424, the contents of which are hereby incorporated by reference in their entireties. In certain embodiments, the antibody or antiserum is an antibody or antiserum that reacts with a non-contiguous epitope (i.e., not contiguous in primary sequence) that is formed by the tertiary or quaternary structure of a neuraminidase.

When designing a recombinant neuraminidase, care should be taken to maintain the stability of the resulting protein. In this regard, it is recommended that cysteine residues capable of forming disulfide bonds be maintained since they contribute to the stability of the neuraminidase protein. See, e.g., Basler et al., 1999, Journal of Virology, 73(10):8095-8103 for non-limiting examples of influenza virus neuraminidase cysteine residues capable of forming disulfide bonds. The stability of a recombinant neuraminidase described herein can be assessed using techniques known in the art, such as sensitivity of the neuraminidase molecules to Ca²⁺, as described in, e.g., Baker and Gandhi, 1976, Archives of Virology, 52:7-18. The stability of a recombinant neuraminidase may be assessed by any method described herein (e.g., in Section 6, infra).

### 5.1.2 Recombinant Neuraminidase with Tetramerization Domain

In one aspect, provided herein are recombinant neuraminidases comprising an influenza virus neuraminidase globular head domain and a tetramerization domain. In a specific embodiment, provided herein is a recombinant neuraminidase comprising an influenza virus neuraminidase globular head domain and a tetramerization domain, wherein the recombinant neuraminidase lacks influenza virus neuraminidase transmembrane and cytoplasmic domains. In specific embodiments, the recombinant neuraminidase does not have any transmembrane domain. In certain embodiments, the recombinant neuraminidase includes a stalk domain or a fragment thereof of the same influenza virus neuraminidase as the globular head domain. The fragment of the stalk domain of the influenza virus neuraminidase may consist of or comprise 2, 3, 4, 5, 6, 7, 8, 9, 10, 11, 12, 13, 14, 15, 16, 17, 18, 19, 20, 25, 30, 35, 40, 45, or 50 amino acid residues of the stalk domain of the influenza virus neuraminidase. Alternatively, the fragment of the stalk domain of the influenza virus neuraminidase may consist of or comprise 2 to 5, 5 to 10, 5 to 15, 10 to 15, 5 to 20, 10 to 20, 15 to 20, 20 to 30, 20 to 40, 25 to 30, 25 to 40, 25 to 45, 25 to 50, 30 to 40, or 40 to 50 amino acid residues of the stalk domain of the influenza virus neuraminidase. In specific embodiments, the recombinant neuraminidase does not include the stalk domain of an influenza virus neuraminidase.

Those of skill in the art will recognize that the delineation of the domains of an influenza virus neuraminidase may be determined from, e.g., crystal structure and/or by using structure prediction software (for example, the website for the Center for Biological Sequence Analysis, Technical University of Denmark DTU, or Pymol) in conjunction with protein alignments. In a specific embodiment, the first cysteine of the globular head domain of influenza virus neuraminidase corresponds to the amino acid residue indicated by the bold and underlined asterisk in FIGS. 9A-9D.

In a specific embodiment, provided herein is a recombinant neuraminidase comprising an influenza virus neuraminidase globular head domain and a tetramerization domain, wherein the recombinant neuraminidase lacks influenza virus neuraminidase stalk, transmembrane and cytoplasmic domains. In specific embodiments, the recombinant neuraminidase does not include any transmembrane. In certain embodiments, the tetramerization domain comprises the tetramerization domain from SEPPALLATA-like MADS domain transcription factor from Arabidopsis thaliana (SMDTF), PiLZ structure from Xhantomonas campestris, or Dictyocaulus viviparus ACE tetramerization domain. In other embodiments, the tetramerization domain comprises a tetramerization domain from a paramyxovirus phosphoprotein (e.g., a Nipah virus phosphoprotein, a Hendra virus phosphoprotein, a respiratory syncytial virus phosphoprotein, human parainfluenza virus (hPIV) phosphoprotein, bovine parainfluenza virus phosphoprotein, a mumps virus phosphoprotein, a Cedar virus phosphoprotein, a Ghana virus phosphoprotein, a Newcastle disease virus phosphoprotein, a canine distemper virus phosphoprotein, or a Peste des petits ruminants virus (PPRV) phosphoprotein). In certain embodiments, the tetramerization domain is not a GCN4 leucine zipper, a bacterial tetrabrachion tetramerization domain or the human vasodilator stimulated phosphoprotein (VASP) tetramerization domain. In a specific embodiment, the tetramerization domain is a tetramerization from a measles virus phosphoprotein tetramerization domain or a Sendai virus phosphoprotein tetramerization domain. In another specific embodiment, the tetramerization domain comprises the amino acid sequence of SEQ ID NO: 1, 2, 3, 4 or 5.

In a particular embodiment, provided herein is a recombinant neuraminidase comprising an influenza virus neuraminidase globular head domain and a tetramerization domain, wherein the recombinant neuraminidase lacks influenza virus neuraminidase stalk, transmembrane and cytoplasmic domains, and wherein the tetramerization domain comprises a measles virus phosphoprotein tetramerization domain or a Sendai virus phosphoprotein tetramerization domain. In a specific embodiment, provided herein is a recombinant neuraminidase comprising or consisting of the amino acid sequence of SEQ ID NO: 24, 25, 26, 27 or 28. In another specific embodiment, provided herein is a recombinant neuraminidase comprising or consisting of the amino acid sequence of SEQ ID NO: 24, 25, 26, 27 or 28 without the signal sequence, histidine tag or both.

In another embodiment, provided herein is a recombinant neuraminidase comprising or consisting of the amino acid sequence of SEQ ID NO: 27, 56, 58, 60 or 62. In another embodiment, provided herein is a recombinant neuraminidase comprising or consisting of the amino acid sequence of SEQ ID NO: 27, 56, 58, 60 or 62 without the signal sequence, histidine tag or both. In a specific embodiment, provided herein is a recombinant neuraminidase comprising or consisting of the amino acid sequence of SEQ ID NO: 27, 56 or 58. In another specific embodiment, provided herein is a recombinant neuraminidase comprising or consisting of the amino acid sequence of SEQ ID NO: 27 without one, two or all of the following: the signal sequence, histidine tag or thrombin cleavage site. In certain embodiments, a recombinant neuraminidase provided herein comprises or consists of the amino acid sequence that is encoded by the nucleotide sequence set forth in SEQ ID NO: 55, 57, 59 or 61.

In certain embodiments, an influenza virus neuraminidase is a human influenza virus neuraminidase. Human influenza virus neuraminidases are known in the art. In certain embodiments, an influenza virus neuraminidase is a swine influenza virus neuraminidase. Swine influenza virus neuraminidases are known in the art. In certain embodiments, an influenza virus neuraminidase is an equine influenza virus neuraminidase. Equine influenza virus neuraminidases are known in the art. In certain embodiments, an influenza virus neuraminidase is an avian influenza virus neuraminidase. Avian influenza virus neuraminidases are known in the art. In certain embodiments, an influenza virus neuraminidase is a seal influenza virus neuraminidase. Seal influenza virus neuraminidases are known in the art.

In some embodiments, an influenza virus neuraminidase is an influenza A virus neuraminidase. In specific embodiments, an influenza virus neuraminidase is an N1, N2, N3, N4, N5, N6, N7, N8 or N9 subtype. Examples of specific influenza A virus neuraminidases include the neuraminidase of an influenza A virus strains described herein. In a specific embodiment, the influenza A virus neuraminidase is influenza A/Brisbane/02/2018 (H1N1)pdm09-like virus neuraminidase, influenza A/Kansas/14/2017 (H3N2)-like virus neuraminidase, influenze A/Brisbane/02/2018 (H1N1)pdm09-like virus neuraminidase, or influenza A/South Australia/34/2019 (H3N2)-like virus neuraminidase.

In some embodiments, an influenza virus neuraminidase is an influenza B virus neuraminidase. In certain embodiments, an influenza B virus neuraminidase is a human influenza B virus neuraminidase. Human influenza B virus neuraminidases are known in the art. In certain embodiments, an influenza B virus neuraminidase is a seal influenza B virus neuraminidase. Seal influenza B virus neuraminidases are known in the art. Examples of specific influenza B virus neuraminidases include the neuraminidase of an influenza B/Yamagata/16/88-lineage virus or an influenza virus B/Victoria/2/87-lineage virus. In a specific embodiment, the influenza B virus neuraminidase is influenza a B/Colorado/06/2017-like virus (B/Victoria/2/87 lineage) neuraminidase, influenza B/Phuket/3073/2013-like virus (B/Yamagata/16/88 lineage) neuraminidase, influenza B/Washington/02/2019-like (B/Victoria lineage) virus neuraminidase, or influenza B/Phuket/3073/2013-like (B/Yamagata lineage) virus neuraminidase.

GenBank™ Accession No. AAA43397.1 provides an exemplary amino acid sequence for a human influenza virus neuraminidase. GenBank™ Accession No. ABG23658.1 (GI: 108946273), GenBank™ Accession No. NP_040981.1 (GI: 8486128), GenBank™ Accession No. AAA43412.1 (GI: 324508), GenBank™ Accession No. ABE97720.1 (GI: 93008579), GenBank™ Accession No. ABE97719.1 (GI: 93008577), and GenBank™ Accession No. ABE97718.1 (GI: 93008575) provide exemplary amino acid sequences for human influenza virus neuraminidases. GenBank™ Accession No. CRI06477.1 provides an exemplary amino acid sequence for a swine influenza virus neuraminidase. GenBank™ Accession No. AAQ90293.1 provides an exemplary amino acid sequence for an equine influenza virus neuraminidase. GenBank™ Accession No. AEX30531.1 (GI: 371449652), GenBank™ Accession No. AEX30532.1 (GI: 371449654), GenBank™ Accession No. AIA62041.1 (GI: 641454926), GenBank™ Accession No. AII30325.1 (GI: 670605039), GenBank™ Accession No. AGO18161.1 (GI: 513130855), and GenBank™ Accession No. AAS89005.1 (GI: 46360357) provide exemplary amino acid sequences for avian influenza virus neuraminidases. Sequences of influenza virus genes may also be found in the Influenza Research Database. For example, influenza virus neuraminidase sequences may be found in the Influenza Research Database under Accession No. FJ66084 and Accession No. KF90392.

In certain embodiments, an influenza virus neuraminidase globular head domain has 70%, 75%, 80%, or 85% identity to the globular head domain of an influenza A virus neuraminidase described herein (e.g., SEQ ID NO: 64 or 66) or known in the art. In some embodiments, an influenza virus neuraminidase globular head domain has 90%, 95%, or 98% identity to the globular head domain of an influenza A virus neuraminidase described herein (e.g., SEQ ID NO: 64 or 66) or known in the art. In certain embodiments, an influenza virus neuraminidase globular head domain has 70%, 75%, 80%, or 85% identity to the globular head domain of an influenza B virus neuraminidase described herein (e.g., SEQ ID NO:65) or known in the art. In some embodiments, an influenza virus neuraminidase globular head domain has 90%, 95%, or 98% identity to the globular head domain of an influenza B virus neuraminidase described herein (e.g., SEQ ID NO:65) or known in the art. In some embodiments, an influenza virus neuraminidase globular head domain comprises or consists of the amino acid sequence of SEQ ID NO:64, 65 or 66.

In certain embodiments, a recombinant neuraminidase provided herein further comprise one or more polypeptide domains. Useful polypeptide domains include domains that facilitate purification, folding and cleavage of portions of a polypeptide. For example, a His tag (His-His-His-His-His-His) (SEQ ID NO:50), FLAG epitope or other purification tag can facilitate purification of an neuraminidase polypeptide provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. Cleavage sites can be used to facilitate cleavage of a portion of a polypeptide, for example cleavage of a purification tag. Useful cleavage sites include a thrombin cleavage site, for example one with the sequence LVPRGSP (SEQ ID NO:51). In a specific embodiment, the cleavage site comprises the amino acid sequence SLVPRGSPSR (SEQ ID NO:63). In certain embodiments, the cleavage site is a cleavage site recognized by Tobacco Etch Virus (TEV) protease (e.g., amino acid sequence Glu-Asn-Leu-Tyr-Phe-Gln-(Gly/Ser) (SEQ ID NO:52)). In certain embodiments, a recombinant neuraminidase provided herein does not include a cleavage site. In some embodiments, a recombinant neuraminidase provided herein comprises a signal peptide and a tag (e.g., a histidine tag). In certain embodiments, a recombinant neuraminidase provided herein comprises a signal peptide, a tag (e.g., a histidine tag), and a cleavage site (e.g., a cleavage site described herein).

In a specific embodiment, a recombinant neuraminidase provided herein includes in order a signal peptide, histidine tag or other purification tag, a tetramerization domain (e.g., MPP or another tetramerization domain described herein or known to one of skill in the art), a cleavage site (e.g., SEQ ID NO:63 or another cleavage site described herein or known to one of skill in the art), and a globular head domain of an influenza virus NA. In another specific embodiment, a recombinant neuraminidase provided herein includes in order a signal peptide, histidine tag or other purification tag, a tetramerization domain (e.g., MPP or another tetramerization domain described herein or known to one of skill in the art), and a globular head domain of an influenza virus NA. In another specific embodiment, a recombinant neuraminidase provided herein includes in order a signal peptide, a tetramerization domain (e.g., MPP or another tetramerization domain described herein or known to one of skill in the art), and a globular head domain of an influenza virus NA. In another specific embodiment, a recombinant neuraminidase described herein comprises the components of a signal peptide, histidine tag or other purification tag, a tetramerization domain (e.g., MPP or another tetramerization domain described herein or known to one of skill in the art), a cleavage site (e.g., SEQ ID NO:63 or another cleavage site described herein or known to one of skill in the art), and a globular head domain of an influenza virus NA in the order described in Section 6, infra. In another specific embodiment, a recombinant neuraminidase described herein comprises the components of a signal peptide, histidine tag or other purification tag, a tetramerization domain (e.g., MPP or another tetramerization domain described herein or known to one of skill in the art), and a globular head domain of an influenza virus NA in the order described in Section 6, infra. In another specific embodiment, a recombinant neuraminidase described herein comprises the components of a signal peptide, a tetramerization domain (e.g., MPP or another tetramerization domain described herein or known to one of skill in the art), and a globular head domain of an influenza virus NA in the order described in Section 6, infra.

In certain embodiments, a recombinant neuraminidase provided herein exists in one, two, three or all of the following forms: monomeric, dimeric, trimeric or tetrameric. In specific embodiments, a recombinant neuraminidase provided herein is tetrameric as assessed by techniques known in the art or described herein.

In specific embodiments, a recombinant neuraminidase provided herein are capable of forming a three-dimensional structure that is similar to the three-dimensional structure of a native influenza neuraminidase. Structural similarity might be evaluated based on any technique deemed suitable by those of skill in the art. For instance, reaction, e.g. under non-denaturing conditions, of a recombinant neuraminidase with a neutralizing antibody or antiserum that recognizes a native influenza neuraminidase might indicate structural similarity. Useful neutralizing antibodies or antisera are described in, e.g., Shoji et al., Hum. Vaccines, 2011, 7:199-204, Wan et al., J. Virol. 2013, 87:9290-9300, Doyle et al. Antivir. Res. 2013, 100:567-574, Doyle et al., Biochem. Biophys. Res. Commun. 2013, 441:226-229, and Wohlbold et al., 2017, Nat.. Microbiol. 2(10): 1415-1424, the contents of which are hereby incorporated by reference in their entireties. In certain embodiments, the antibody or antiserum is an antibody or antiserum that reacts with a non-contiguous epitope (i.e., not contiguous in primary sequence) that is formed by the tertiary or quaternary structure of a neuraminidase.

When designing a recombinant neuraminidase, care should be taken to maintain the stability of the resulting protein. In this regard, it is recommended that cysteine residues capable of forming disulfide bonds be maintained since they contribute to the stability of the neuraminidase protein. See, e.g., Basler et al., 1999, Journal of Virology, 73(10):8095-8103 for non-limiting examples of influenza virus neuraminidase cysteine residues capable of forming disulfide bonds. The stability of a recombinant neuraminidase described herein can be assessed using techniques known in the art, such as sensitivity of the neuraminidase molecules to Ca²⁺, as described in, e.g., Baker and Gandhi, 1976, Archives of Virology, 52:7-18. The stability of a recombinant neuraminidase may be assessed by any method described herein (e.g., in Section 6, infra).

### 5.2 Nucleic Acid Sequences

Provided herein are nucleic acid sequences that encode recombinant neuraminidase described herein. Due to the degeneracy of the genetic code, any nucleic acid sequence that encodes a recombinant neuraminidase described herein is encompassed herein. In specific embodiments, provided herein is a nucleic acid sequence comprising a nucleotide sequence encoding a recombinant neuraminidase (with or without a signal peptide). In certain embodiment, the nucleotide sequence encoding the recombinant neuraminidase comprises a nucleotide sequence encoding a signal peptide (e.g., a signal peptide/membrane anchor from the NA of the same influenza virus as the influenza virus engineered to express the recombinant neuraminidase polypeptide). In some embodiments, the nucleic acid sequence further comprises the 5′ non-coding region and 3′ non-coding region of an influenza virus NA (e.g., the 5′ non-coding region and 3′ non-coding region from the NA of the same influenza virus as the influenza virus engineered to express the recombinant neuraminidase). In certain embodiments, the nucleic acid sequence further comprises the packaging signals of an influenza virus NA gene segment. In certain embodiments, the nucleic acid sequence further comprises the packaging signals of an influenza virus gene segment other than an influenza virus NA gene segment, such as described in, e.g., International Patent Application Publication No. WO 2011/014645; Gao & Palese 2009, PNAS 106:15891-15896; U.S. Pat. No. 8,828,406, each of which is incorporated herein in its entirety. In some embodiments, the nucleic acid sequences provided herein are codon optimized.

In certain embodiments, provided herein is a nucleic acid sequence comprising the nucleotide sequence sequence set forth in SEQ ID NO: 55, 57, 59 or 61. In a specific embodiment, provided herein is a nucleic acid sequence comprising the nucleotide sequence set forth in SEQ ID NO: 55 or 57.

Also provided herein are nucleic acid sequences capable of hybridizing to a nucleic acid encoding a recombinant neuraminidase. In some embodiments, provided herein is a nucleic acid sequence capable of hybridizing to the nucleotide sequence set forth in SEQ ID NO:55, 57, 60 or 61. In certain embodiments, provided herein are nucleic acid sequences capable of hybridizing to a fragment of a nucleic acid sequence encoding a recombinant neuraminidase. In some embodiments, provided herein is a nucleic acid sequence capable of hybridizing to a fragment (e.g., comprising or consisting of 250, 300, 350, 400, 450, 500, 550, 600 or more nucleotides, or between 300 to 600, 400 to 600 or 500 to 700 nucleotides) of SEQ ID NO: 55, 57, 60 or 61. In other embodiments, provided herein are nucleic acid sequences capable of hybridizing to the full length of a nucleic acid sequence encoding a recombinant neuraminidase. General parameters for hybridization conditions for nucleic acids are described in Sambrook et al., Molecular Cloning—A Laboratory Manual (2nd Ed.), Vols. 1-3, Cold Spring Harbor Laboratory, Cold Spring Harbor, New York (1989), and in Ausubel et al., Current Protocols in Molecular Biology, vol. 2, Current Protocols Publishing, New York (1994). Hybridization may be performed under high stringency conditions, medium stringency conditions, or low stringency conditions. Those of skill in the art will understand that low, medium and high stringency conditions are contingent upon multiple factors all of which interact and are also dependent upon the nucleic acids in question. For example, high stringency conditions may include temperatures within 5° C. melting temperature of the nucleic acid(s), a low salt concentration (e.g., less than 250 mM), and a high co-solvent concentration (e.g., 1-20% of co-solvent, e.g., DMSO). Low stringency conditions, on the other hand, may include temperatures greater than 10° C. below the melting temperature of the nucleic acid(s), a high salt concentration (e.g., greater than 1000 mM) and the absence of co-solvents.

In some embodiments, a nucleic acid sequence comprising a nucleotide sequence encoding a recombinant neuraminidase is isolated. In certain embodiments, an “isolated” nucleic acid sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences in which the nucleic acid sequence is separated from cellular components of the cells from which it is isolated or recombinantly produced. Thus, nucleic acid sequence that is substantially free of cellular material includes preparations of nucleic acid sequence having less than about 30%, 20%, 10%, or 5% (by dry weight) of other nucleic acids. The term “substantially free of culture medium” includes preparations of nucleic acid sequence in which the culture medium represents less than about 50%, 20%, 10%, or 5% of the volume of the preparation. The term “substantially free of chemical precursors or other chemicals” includes preparations in which the nucleic acid sequence is separated from chemical precursors or other chemicals which are involved in the synthesis of the nucleic acid sequence. In specific embodiments, such preparations of the nucleic acid sequence have less than about 50%, 30%, 20%, 10%, 5% (by dry weight) of chemical precursors or compounds other than the nucleic acid sequence of interest.

### 5.3 Expression of Recombinant Neuraminidase

Provided herein are vectors, including expression vectors, containing a nucleic acid sequence comprising a nucleotide sequence encoding a recombinant neuraminidase (NA) described herein. In a specific embodiment, the vector is an expression vector that is capable of directing the expression of a nucleic acid sequence encoding a recombinant neuraminidase. Non-limiting examples of expression vectors include, but are not limited to, plasmids and viral vectors, such as replication defective retroviruses, adenoviruses, vesicular stomatitis virus (VSV), Newcastle disease virus (NDV), vaccinia (e.g., Modified Vaccinia Ankara virus), adeno-associated viruses and baculoviruses. Techniques known to one of skill in the art may be used to engineer such viral vectors to express a recombinant neuraminidase described herein. Expression vectors also may include, without limitation, transgenic animals and non-mammalian cells/organisms, e.g., non-mammalian cells/organisms that have been engineered to perform mammalian N-linked glycosylation.

In some embodiments, provided herein are expression vectors encoding components of a recombinant neuraminidase (e.g., the stalk domain and the globular head domain, or portions of either domain). Such vectors may be used to express the components in one or more host cells and the components may be isolated and conjugated together with a linker using techniques known to one of skill in the art.

An expression vector comprises a nucleic acid sequence comprising a nucleotide sequence encoding a recombinant neuraminidase described herein and in a form suitable for expression of the nucleic acid sequence in a host cell. In a specific embodiment, an expression vector includes one or more regulatory sequences, selected on the basis of the host cells to be used for expression, which is operably linked to the nucleic acid sequence to be expressed. Within an expression vector, “operably linked” is intended to mean that a nucleic acid sequence of interest is linked to the regulatory sequence(s) in a manner which allows for expression of the nucleic acid sequence (e.g., in an in vitro transcription/translation system or in a host cell when the vector is introduced into the host cell). Regulatory sequences include promoters, enhancers and other expression control elements (e.g., polyadenylation signals). Regulatory sequences include those which direct constitutive expression of a nucleic acid sequence in many types of host cells, those which direct expression of the nucleic acid sequence only in certain host cells (e.g., tissue-specific regulatory sequences), and those which direct the expression of the nucleic acid upon stimulation with a particular agent (e.g., inducible regulatory sequences). It will be appreciated by those skilled in the art that the design of the expression vector can depend on such factors as the choice of the host cell to be transformed, the level of expression of protein desired, etc. The term “host cell” is intended to include a particular subject cell transformed or transfected with a nucleic acid sequence and the progeny or potential progeny of such a cell. Progeny of such a cell may not be identical to the parent cell transformed or transfected with the nucleic acid sequence due to mutations or environmental influences that may occur in succeeding generations or integration of the nucleic acid sequence into the host cell genome. In specific embodiments, the host cell is a cell line. Examples of host cells (e.g., yeat, avian, insect, plant and/or mammalian cells) that may be used to express a nucleic acid sequence are provided herein.

Expression vectors can be designed for expression of a recombinant neuraminidase described herein using prokaryotic (e g., E. coli) or eukaryotic cells (e.g., insect cells (using baculovirus expression vectors, see, e.g., Treanor et al., 2007, JAMA, 297(14):1577-1582 incorporated by reference herein in its entirety), yeast cells, plant cells, algae, avian, or mammalian cells). Examples of yeast host cells include, but are not limited to S. pombe and S. cerevisiae and examples, infra. An example of avian cells includes, but is not limited to EB66 cells. Examples of mammalian host cells include, but are not limited to, A549 cells, Crucell Per.C6 cells, Vero cells, CHO cells, VERO cells, BHK cells, HeLa cells, COS cells, MDCK cells, 293 cells, 3T3 cells or WI38 cells. In certain embodiments, the hosts cells are myeloma cells, e.g., NS0 cells, 45.6 TG1.7 cells, AF-2 clone 9B5 cells, AF-2 clone 9B5 cells, J558L cells, MOPC 315 cells, MPC-11 cells, NCI-H929 cells, NP cells, NS0/1 cells, P3 NS1 Ag4 cells, P3/NS1/1-Ag4-1 cells, P3U1 cells, P3X63Ag8 cells, P3X63Ag8.653 cells, P3X63Ag8U.1 cells, RPMI 8226 cells, Sp20-Ag14 cells, U266B1 cells, X63AG8.653 cells, Y3.Ag.1.2.3 cells, and YO cells. Non-limiting examples of insect cells include Sf9, Sf21, Trichoplusia ni, Spodoptera frugiperda and Bombyx mori. In a particular embodiment, a mammalian cell culture system (e.g. Chinese hamster ovary or baby hamster kidney cells) is used for expression of a recombinant neuraminidase. In another embodiment, a plant cell culture system is used for expression of a recombinant neuraminidase. See, e.g., U.S. Pat. Nos. 7,504,560; 6,770,799; 6,551,820; 6,136,320; 6,034,298; 5,914,935; 5,612,487; and 5,484,719, and U.S. patent application publication Nos. 2009/0208477, 2009/0082548, 2009/0053762, 2008/0038232, 2007/0275014 and 2006/0204487 for plant cells and methods for the production of proteins utilizing plant cell culture systems. In specific embodiments, plant cell culture systems are not used for expression of a recombinant neuraminidase. The host cells comprising a nucleic acid sequence that encodes a recombinant neuraminidase described herein can be isolated, i.e., the cells are outside of the body of a subject. In certain embodiments, the cells are engineered to express a nucleic acid sequence that encodes a recombinant neuraminidase described herein. In particular embodiments, the cells are engineered to express a recombinant neuraminidase described herein. In specific embodiments, the host cells are cells from a cell line.

In certain embodiments, provided herein is host cell(s) comprising a nucleic acid sequence that comprises a nucleotide sequence encoding a recombinant neuraminidase described herein. In some embodiments, provided herein is a host cell(s) engineered to express a express a recombinant neuraminidase described herein. Host cells include those cells, including cell lines, described herein.

An expression vector can be introduced into host cells via conventional transformation or transfection techniques. Such techniques include, but are not limited to, calcium phosphate or calcium chloride co-precipitation, DEAE-dextran-mediated transfection, lipofection, and electroporation. Suitable methods for transforming or transfecting host cells can be found in Sambrook et al., 1989, Molecular Cloning—A Laboratory Manual, 2nd Edition, Cold Spring Harbor Press, New York, and other laboratory manuals. In certain embodiments, a host cell is transiently transfected with an expression vector containing a nucleic acid sequence encoding a recombinant neuraminidase. In other embodiments, a host cell is stably transfected with an expression vector containing a nucleic acid sequence encoding a recombinant neuraminidase.

For stable transfection of mammalian cells, it is known that, depending upon the expression vector and transfection technique used, only a small fraction of cells may integrate the foreign DNA into their genome. In order to identify and select these integrants, a nucleic acid that encodes a selectable marker (e.g., for resistance to antibiotics) is generally introduced into the host cells along with the nucleic acid of interest. Examples of selectable markers include those which confer resistance to drugs, such as G418, hygromycin and methotrexate. Cells stably transfected with the introduced nucleic acid sequence can be identified by drug selection (e.g., cells that have incorporated the selectable marker gene will survive, while the other cells die).

As an alternative to recombinant expression of a recombinant neuraminidase using a host cell, an expression vector containing a nucleic acid sequence encoding a recombinant neuraminidase can be transcribed and translated in vitro using, e.g., T7 promoter regulatory sequences and T7 polymerase. In a specific embodiment, a coupled transcription/translation system, such as Promega TNT®, or a cell lysate or cell extract comprising the components necessary for transcription and translation may be used to produce a recombinant neuraminidase.

Once a recombinant neuraminidase has been produced, it may be isolated or purified by any method known in the art for isolation or purification of a protein, for example, by chromatography (e.g., ion exchange, affinity, particularly by affinity for the specific antigen, by Protein A, and sizing column chromatography), centrifugation, differential solubility, or by any other standard technique for the isolation or purification of proteins.

Accordingly, provided herein are methods for producing a recombinant neuraminidase. In one embodiment, the method comprises culturing a host cell containing a nucleic acid sequence comprising a nucleotide sequence encoding a recombinant neuraminidase in a suitable medium such that the recombinant neuraminidase is produced. In some embodiments, the method further comprises isolating the recombinant neuraminidase from the medium or the host cell.

Also provided herein are methods for producing a virus (e.g., an influenza virus (see Section 5.4, infra) or a non-influenza virus vector (e.g., a baculovirus) comprising a recombinant neuraminidase described herein, comprising propagating the virus in any substrate that allows the virus to grow to titers that permit their use in accordance with the methods described herein. In one embodiment, the substrate allows the viruses to grow to titers comparable to those determined for the corresponding wild-type viruses. In a specific embodiment, the virus is propagated in embryonated eggs (e.g., chicken eggs). In a specific embodiment, the virus is propagated in 8 day old, 9-day old, 8-10 day old, 10 day old, 11-day old, 10-12 day old, or 12-day old embryonated eggs (e.g., chicken eggs). In some embodiments, the virus is propagated in embryonated eggs (e.g., chicken eggs) that are interferon (IFN)-deficient. In some embodiments, the virus is propagated in embryonated eggs (e.g., chicken eggs) that are impaired in interferon (IFN) expression. In certain embodiments, the virus is propagated in MDCK cells, Vero cells, 293T cells, or other cell lines known in the art. See, e.g., Section 5.3, supra, for examples of cell lines. In certain embodiments, the virus is propagated in cells derived from embryonated eggs. In certain embodiments, the virus is propagated in an embryonated egg (e.g., chicken eggs) and then in MDCK cells, Vero cells, 293T cells, or other cell lines known in the art.

### 5.4 Influenza Viruses

In one aspect, provided herein are influenza viruses containing a recombinant neuraminidase (NA) described herein. In specific embodiments, the influenza viruses described are recombinantly produced. In a specific embodiment, a recombinant neuraminidase is incorporated into the virion of the influenza virus. The influenza viruses may be conjugated to moieties that target the viruses to particular cell types, such as immune cells. In some embodiments, the virions of the influenza virus have incorporated into them or express a heterologous polypeptide in addition to a recombinant neuraminidase. The heterologous polypeptide may be a polypeptide that has immunopotentiating activity, or that targets the influenza virus to a particular cell type, such as an antibody that binds to an antigen on a specific cell type or a ligand that binds a specific receptor on a specific cell type.

Influenza viruses containing a recombinant neuraminidase may be produced by supplying in trans the recombinant neuraminidase during production of virions using techniques known to one skilled in the art, such as reverse genetics and helper-free plasmid rescue. Alternatively, the replication of a parental influenza virus comprising a genome engineered to express a recombinant neuraminidase in cells susceptible to infection with the virus. In certain embodiments, the neuraminidase function is provided in trans to produce progeny influenza viruses.

In another aspect, provided herein are influenza viruses comprising a genome engineered to express a recombinant neuraminidase. In a specific embodiment, the genome of a parental influenza virus is engineered to encode a recombinant neuraminidase, which is expressed by progeny influenza virus. In another specific embodiment, the genome of a parental influenza virus is engineered to encode a recombinant neuraminidase, which is expressed and incorporated into the virions of progeny influenza virus. Thus, the progeny influenza virus resulting from the replication of the parental influenza virus contain a recombinant neuraminidase. In specific embodiments, the parental influenza virus is an influenza A virus. In other specific embodiments, the parental influenza virus is an influenza B virus.

In some embodiments, the virions of the parental influenza virus have incorporated into them a heterologous polypeptide. In certain embodiments, the genome of a parental influenza virus is engineered to encode a heterologous polypeptide and a recombinant neuraminidase, which are expressed by progeny influenza virus. In specific embodiments, the recombinant neuraminidase, the heterologous polypeptide or both are incorporated into virions of the progeny influenza virus.

Since the genome of influenza A and B viruses consist of eight (8) single-stranded, negative sense segments, the genome of a parental influenza virus may be engineered to express a recombinant neuraminidase (and any other polypeptide, such as a heterologous polypeptide) using a recombinant segment and techniques known to one skilled in the art, such a reverse genetics and helper-free plasmid rescue. In one embodiment, provided herein is a recombinant segment comprising a nucleic acid sequence encoding a recombinant neuraminidase, as well as the 3′ and 5′ incorporation signals which are required for proper replication, transcription and packaging of the vRNAs (Fujii et al., 2003, Proc. Natl. Acad. Sci. USA 100:2002-2007; Zheng, et al., 1996, Virology 217:242-251, International Publication No. WO 2011/014645, all of which are incorporated by reference herein in their entireties). In a specific embodiment, the recombinant segment uses the 3′ and 5′ noncoding and/or nontranslated sequences of segments of influenza viruses that are from a different or the same type, subtype/lineage or strain as the parental influenza virus. In some embodiments, the recombinant segment comprises the 3′ noncoding region of an influenza virus NA, the untranslated regions of an influenza virus NA, and the 5′ non-coding region of an influenza virus NA. In specific embodiments, the recombinant segment comprises packaging signals, such as the 5′ and 3′ non-coding regions of the NA segment of an influenza virus, from the same type, lineage, or strain as the influenza virus backbone. For example, if the recombinant neuraminidase is engineered to be expressed from an influenza A virus, then the nucleotide sequence encoding the recombinant neuraminidase (NA) comprises the 5′ and 3′ non-coding regions of the NA segment of the influenza A virus. In certain embodiments, the recombinant segment encoding the recombinant neuraminidase may replace the NA segment of a parental influenza virus.

In some embodiments, an NA gene segment encodes a recombinant neuraminidase. In specific embodiments, the influenza virus NA gene segment and at least one other influenza virus gene segment comprise packaging signals that enable the influenza virus NA gene segment and at least one other gene segment to segregate together during replication of a recombinant influenza virus (see, Gao & Palese 2009, PNAS 106:15891-15896; U.S. Pat. No. 8,828,406; and International Application Publication No. WO11/014645, each of which is incorporated herein by reference in its entirety).

In some embodiments, the genome of a parental influenza virus may be engineered to express a recombinant neuraminidase using a recombinant segment that is bicistronic. Bicistronic techniques allow the engineering of coding sequences of multiple proteins into a single mRNA through the use of internal ribosome entry site (IRES) sequences. IRES sequences direct the internal recruitment of ribosomes to the RNA molecule and allow downstream translation in a cap independent manner. Briefly, a coding region of one protein is inserted into the open reading frame (ORF) of a second protein. The insertion is flanked by an IRES and any untranslated signal sequences necessary for proper expression and/or function. The insertion must not disrupt the ORF, polyadenylation or transcriptional promoters of the second protein (see, e.g., Garcia-Sastre et al., 1994, J. Virol. 68:6254-6261 and Garcia-Sastre et al., 1994 Dev. Biol. Stand. 82:237-246, each of which is hereby incorporated by reference in its entirety). See also, e.g., U.S. Pat. Nos. 6,887,699, 6,001,634, 5,854,037 and 5,820,871, each of which is incorporated herein by reference in its entirety. Any IRES known in the art or described herein may be used in accordance with the invention (e.g., the TRES of BiP gene, nucleotides 372 to 592 of GenBank database entry HUMGRP78; or the IRES of encephalomyocarditis virus (EMCV), nucleotides 1430-2115 of GenBank database entry CQ867238.). Thus, in certain embodiments, a parental influenza virus is engineered to contain a bicistronic RNA segment that expresses the recombinant neuraminidase and another polypeptide, such as a gene expressed by the parental influenza virus. In some embodiments, the parental influenza virus gene is the NA gene.

Techniques known to one skilled in the art may be used to produce an influenza virus containing a recombinant neuraminidase and an influenza virus comprising a genome engineered to express a recombinant neuraminidase. For example, reverse genetics techniques may be used to generate such an influenza virus. Briefly, reverse genetics techniques generally involve the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in European Patent Publication EP 0702085A1, published Feb. 20, 1996; in U.S. patent application Ser. No. 09/152,845; in International Patent Publications PCT WO 97/12032 published Apr. 3, 1997; WO 96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

Alternatively, helper-free plasmid technology may be used to produce an influenza virus containing a recombinant neuraminidase and an influenza virus comprising a genome engineered to express a recombinant neuraminidase. Briefly, full length cDNAs of viral segments are amplified using PCR with primers that include unique restriction sites, which allow the insertion of the PCR product into the plasmid vector (Flandorfer et al., 2003, J. Virol. 77:9116-9123; Nakaya et al., 2001, J. Virol. 75:11868-11873; both of which are incorporated herein by reference in their entireties). The plasmid vector is designed so that an exact negative (vRNA sense) transcript is expressed. For example, the plasmid vector may be designed to position the PCR product between a truncated human RNA polymerase I promoter and a hepatitis delta virus ribozyme sequence such that an exact negative (vRNA sense) transcript is produced from the polymerase I promoter. Separate plasmid vectors comprising each viral segment as well as expression vectors comprising necessary viral proteins may be transfected into cells leading to production of recombinant viral particles. In another example, plasmid vectors from which both the viral genomic RNA and mRNA encoding the necessary viral proteins are expressed may be used. For a detailed description of helper-free plasmid technology see, e.g., International Publication No. WO 01/04333; U.S. Pat. Nos. 6,951,754, 7,384,774, 6,649,372, and 7,312,064; Fodor et al., 1999, J. Virol. 73:9679-9682; Quinlivan et al., 2005, J. Virol. 79:8431-8439; Hoffmann et al., 2000, Proc. Natl. Acad. Sci. USA 97:6108-6113; and Neumann et al., 1999, Proc. Natl. Acad. Sci. USA 96:9345-9350, each of which is incorporated herein by reference in its entirety. In a specific embodiment, a method analogous to that described in Section 6 is used to construct a recombinant neuraminidase described herein. In a specific embodiment, a method analogous to that described in Section 6 is used to construct an influenza virus containing and expressing a recombinant neuraminidase. In a specific embodiment, a method analogous to that described in Section 6 is used to construct and propagate a recombinant neuraminidase.

In some embodiments, a recombinant influenza virus is produced by reverse genetics, using a DNA plasmid(s) that expresses a recombinant neuraminidase, which is co-transfected with plasmids for the other 7 genes of influenza virus in a mammalian cell line, such as HEK293T cells. In a specific embodiment, the recombinant influenza virus replicates in embryonated chicken eggs without apparent disadvantages over the influenza viruses that do not have a recombinant neuraminidase described herein. For example, the recombinant influenza virus described herein replicates to comparable titers as the corresponding wild-type influenza virus in a particular cell line.

The influenza viruses described herein may be propagated in any substrate that allows the virus to grow to titers that permit their use in accordance with the methods described herein. Thus, in certain embodiments, provided herein is a method for producing a virus described herein comprising propagating the virus in a substrate. In one embodiment, the substrate allows the viruses to grow to titers comparable to those determined for the corresponding wild-type viruses. In certain embodiments, the substrate is one which is biologically relevant to the influenza virus. In a specific embodiment, an attenuated influenza virus by virtue of, e.g., a mutation in the NS1 gene, may be propagated in an IFN-deficient substrate. For example, a suitable IFN-deficient substrate may be one that is defective in its ability to produce or respond to interferon, or is one which an IFN-deficient substrate may be used for the growth of any number of viruses which may require interferon-deficient growth environment. See, for example, U.S. Pat. Nos. 6,573,079, issued Jun. 3, 2003, U.S. Pat. No. 6,852,522, issued Feb. 8, 2005, and U.S. Pat. No. 7,494,808, issued Feb. 24, 2009, the entire contents of each of which is incorporated herein by reference in its entirety. In a specific embodiment, the virus is propagated in embryonated eggs (e.g., chicken eggs). In a specific embodiment, the virus is propagated in 8 day old, 9-day old, 8-10 day old, 10 day old, 11-day old, 10-12 day old, or 12-day old embryonated eggs (e.g., chicken eggs). In some embodiments, the virus is propagated in embryonated eggs (e.g., chicken eggs) that are IFN-deficient. In certain embodiments, the virus is propagated in MDCK cells, Vero cells, 293T cells, or other cell lines known in the art. See, e.g., Section 5.3, supra, for examples of cell lines. In certain embodiments, the virus is propagated in cells derived from embryonated eggs.

The influenza viruses described herein may be isolated and purified by any method known to those of skill in the art. In one embodiment, the virus is removed from cell culture and separated from cellular components, typically by well known clarification procedures, e.g., such as gradient centrifugation and column chromatography, and may be further purified as desired using procedures well known to those skilled in the art, e.g., plaque assays.

In certain embodiments, the influenza viruses, or influenza virus polypeptides, genes or genome segments for use as described herein are obtained or derived from an influenza A virus. In certain embodiments, the influenza viruses, or influenza virus polypeptides, genes or genome segments for use as described herein are obtained or derived from a single influenza A virus subtype/lineage or strain. In other embodiments, the influenza viruses, or influenza virus polypeptides, genes or genome segments for use as described herein are obtained or derived from two or more influenza A virus subtypes or strains. In a specific embodiment, the influenza A virus is an influenza virus of the H1, H2, H3, H4, H5, H6, H7, H8, H9, H10, H11, H12, H13, H14, H15, H16, H17, or H18 subtype. In a specific embodiment, the influenza A virus is an influenza virus of the H2, H4, H5, H6, H7, H8, H9, H10, H11, H12, H13, H14, H15, H16, H17, or H18 subtype. In a specific embodiment, the influenza A virus is an influenza virus of the H1 or H3 subtype. In a specific embodiment, the influenza A virus is an influenza virus of the H5, H7, H9 or H10 subtype.

Non-limiting examples of influenza A viruses include subtype H10N4, subtype H10N5, subtype H10N8, subtype, H14N5, subtype H10N7, subtype H10N8, subtype H10N9, subtype H11N1, subtype H11N13, subtype H11N2, subtype H11N4, subtype H11N6, subtype H11N8, subtype H11N9, subtype H12N1, subtype H12N4, subtype H12N5, subtype H12N8, subtype H13N2, subtype H13N3, subtype H13N6, subtype H13N7, subtype H14N5, subtype H14N6, subtype H15N8, subtype H15N9, subtype H16N3, subtype H1N1, subtype H1N2, subtype H1N3, subtype H1N6, subtype H1N9, subtype H2N1, subtype H2N2, subtype H2N3, subtype H2N5, subtype H2N7, subtype H2N8, subtype H2N9, subtype H3N1, subtype H3N2, subtype H3N3, subtype H3N4, subtype H3N5, subtype H3N6, subtype H3N8, subtype H3N9, subtype H4N1, subtype H4N2, subtype H4N3, subtype H4N4, subtype H4N5, subtype H4N6, subtype H4N8, subtype H4N9, subtype H5N1, subtype H5N2, subtype H5N3, subtype H5N4, subtype H5N6, subtype H5N7, subtype H5N8, subtype H5N9, subtype H6N1, subtype H6N2, subtype H6N3, subtype H6N4, subtype H6N5, subtype H6N6, subtype H6N7, subtype H6N8, subtype H6N9, subtype H7N1, subtype H7N2, subtype H7N3, subtype H7N4, subtype H7N5, subtype H7N7, subtype H7N8, subtype H7N9, subtype H8N4, subtype H8N5, subtype H9N1, subtype H9N2, subtype H9N3, subtype H9N5, subtype H9N6, subtype H9N7, subtype H9N8, and subtype H9N9.

Specific examples of strains of influenza A virus include, but are not limited to: A/Victoria/361/2011 (H3N2); A/California/4/2009 (H1N1); A/California/7/2009 (H1N1); A/Perth/16/2009 (H3N2); A/Brisbane/59/2007 (H1N1); A/Brisbane/10/2007 (H3N2); A/sw/Iowa/15/30 (H1N1); A/WSN/33 (H1N1); A/eq/Prague/1/56 (H7N7); A/PR/8/34; A/mallard/Potsdam/178-4/83 (H2N2); A/herring gull/DE/712/88 (H16N3); A/sw/Hong Kong/168/1993 (H1N1); A/mallard/Alberta/211/98 (H1N1); A/shorebird/Delaware/168/06 (H16N3); A/sw/Netherlands/25/80 (H1N1); A/sw/Germany/2/81 (H1N1); A/sw/Hannover/1/81 (H1N1); A/sw/Potsdam/1/81 (H1N1); A/sw/Potsdam/15/81 (H1N1); A/sw/Potsdam/268/81 (H1N1); A/sw/Finistere/2899/82 (H1N1); A/sw/Potsdam/35/82 (H3N2); A/sw/Cote d'Armor/3633/84 (H3N2); A/sw/Gent/1/84 (H3N2); A/sw/Netherlands/12/85 (H1N1); A/sw/Karrenzien/2/87 (H3N2); A/sw/Schwerin/103/89 (H1N1); A/turkey/Germany/3/91 (H1N1); A/sw/Germany/8533/91 (H1N1); A/sw/Belgium/220/92 (H3N2); A/sw/Gent/V230/92 (H1N1); A/sw/Leipzig/145/92 (H3N2); A/sw/Re220/92 hp (H3N2); A/sw/Bakum/909/93 (H3N2); A/sw/Schleswig-Holstein/1/93 (H1N1); A/sw/Scotland/419440/94 (H1N2); A/sw/Bakum/5/95 (H1N1); A/sw/Best/5C/96 (H1N1); A/sw/England/17394/96 (H1N2); A/sw/Jena/5/96 (H3N2); A/sw/Oedenrode/7C/96 (H3N2); A/sw/Lohne/1/97 (H3N2); A/sw/Cote d'Armor/790/97 (H1N2); A/sw/Bakum/1362/98 (H3N2); A/sw/Italy/1521/98 (H1N2); A/sw/Italy/1553-2/98 (H3N2); A/sw/Italy/1566/98 (H1N1); A/sw/Italy/1589/98 (H1N1); A/sw/Bakum/8602/99 (H3N2); A/sw/Cotes d'Armor/604/99 (H1N2); A/sw/Cote d'Armor/1482/99 (H1N1); A/sw/Gent/7625/99 (H1N2); A/Hong Kong/1774/99 (H3N2); A/sw/Hong Kong/5190/99 (H3N2); A/sw/Hong Kong/5200/99 (H3N2); A/sw/Hong Kong/5212/99 (H3N2); A/sw/Ille et Villaine/1455/99 (H1N1); A/sw/Italy/1654-1/99 (H1N2); A/sw/Italy/2034/99 (H1N1); A/sw/Italy/2064/99 (H1N2); A/sw/Berlin/1578/00 (H3N2); A/sw/Bakum/1832/00 (H1N2); A/sw/Bakum/1833/00 (H1N2); A/sw/Cote d'Armor/800/00 (H1N2); A/sw/Hong Kong/7982/00 (H3N2); A/sw/Italy/1081/00 (H1N2); A/sw/Belzig/2/01 (H1N1); A/sw/Belzig/54/01 (H3N2); A/sw/Hong Kong/9296/01 (H3N2); A/sw/Hong Kong/9745/01 (H3N2); A/sw/Spain/33601/01 (H3N2); A/sw/Hong Kong/1144/02 (H3N2); A/sw/Hong Kong/1197/02 (H3N2); A/sw/Spain/39139/02 (H3N2); A/sw/Spain/42386/02 (H3N2); A/Switzerland/8808/2002 (H1N1); A/sw/Bakum/1769/03 (H3N2); A/sw/Bissendorf/IDT1864/03 (H3N2); A/sw/Ehren/IDT2570/03 (H1N2); A/sw/Gescher/IDT2702/03 (H1N2); A/sw/Haselünne/2617/03 hp (H1N1); A/sw/Löningen/IDT2530/03 (H1N2); A/sw/IVD/IDT2674/03 (H1N2); A/sw/Nordkirchen/IDT1993/03 (H3N2); A/sw/Nordwalde/IDT2197/03 (H1N2); A/sw/Norden/IDT2308/03 (H1N2); A/sw/Spain/50047/03 (H1N1); A/sw/Spain/51915/03 (H1N1); A/sw/Vechta/2623/03 (H1N1); A/sw/Visbek/IDT2869/03 (H1N2); A/sw/Waltersdorf/IDT2527/03 (H1N2); A/sw/Damme/IDT2890/04 (H3N2); A/sw/Geldern/IDT2888/04 (H1N1); A/sw/Granstedt/IDT3475/04 (H1N2); A/sw/Greven/IDT2889/04 (H1N1); A/sw/Gudensberg/IDT2930/04 (H1N2); A/sw/Gudensberg/IDT2931/04 (H1N2); A/sw/Lohne/IDT3357/04 (H3N2); A/sw/Nortrup/IDT3685/04 (H1N2); A/sw/Seesen/IDT3055/04 (H3N2); A/sw/Spain/53207/04 (H1N1); A/sw/Spain/54008/04 (H3N2); A/sw/Stolzenau/IDT3296/04 (H1N2); A/sw/Wedel/IDT2965/04 (H1N1); A/sw/Bad Griesbach/IDT4191/05 (H3N2); A/sw/Cloppenburg/IDT4777/05 (H1N2); A/sw/Dötlingen/IDT3780/05 (H1N2); A/sw/Dötlingen/IDT4735/05 (H1N2); A/sw/Egglham/IDT5250/05 (H3N2); A/sw/Harkenblek/IDT4097/05 (H3N2); A/sw/Hertzen/IDT4317/05 (H3N2); A/sw/Krogel/IDT4192/05 (H1N1); A/sw/Laer/IDT3893/05 (H1N1); A/sw/Laer/IDT4126/05 (H3N2); A/sw/Merzen/IDT4114/05 (H3N2); A/sw/Muesleringen-S./IDT4263/05 (H3N2); A/sw/Osterhofen/IDT4004/05 (H3N2); A/sw/Sprenge/IDT3805/05 (H1N2); A/sw/Stadtlohn/IDT3853/05 (H1N2); A/sw/Voglarn/IDT4096/05 (H1N1); A/sw/Wohlerst/IDT4093/05 (H1N1); A/sw/Bad Griesbach/IDT5604/06 (H1N1); A/sw/Herzlake/IDT5335/06 (H3N2); A/sw/Herzlake/IDT5336/06 (H3N2); A/sw/Herzlake/IDT5337/06 (H3N2); and A/wild boar/Germany/R169/2006 (H3N2). In a specific embodiment, an influenza A virus is influenza A/Brisbane/02/2018 (H1N1)pdm09-like virus, influenza A/Kansas/14/2017 (H3N2)-like virus, influenze A/Brisbane/02/2018 (H1N1)pdm09-like virus, or influenza A/South Australia/34/2019 (H3N2)-like virus.

Other specific examples of strains of influenza A virus include, but are not limited to: A/Toronto/3141/2009 (H1N1); A/Regensburg/D6/2009 (H1N1); A/Bayern/62/2009 (H1N1); A/Bayern/62/2009 (H1N1); A/Bradenburg/19/2009 (H1N1); A/Bradenburg/20/2009 (H1N1); A/Distrito Federal/2611/2009 (H1N1); A/Mato Grosso/2329/2009 (H1N1); A/Sao Paulo/1454/2009 (H1N1); A/Sao Paulo/2233/2009 (H1N1); A/Stockholm/37/2009 (H1N1); A/Stockholm/41/2009 (H1N1); A/Stockholm/45/2009 (H1N1); A/swine/Alberta/OTH-33-1/2009 (H1N1); A/swine/Alberta/OTH-33-14/2009 (H1N1); A/swine/Alberta/OTH-33-2/2009 (H1N1); A/swine/Alberta/OTH-33-21/2009 (H1N1); A/swine/Alberta/OTH-33-22/2009 (H1N1); A/swine/Alberta/OTH-33-23/2009 (H1N1); A/swine/Alberta/OTH-33-24/2009 (H1N1); A/swine/Alberta/OTH-33-25/2009 (H1N1); A/swine/Alberta/OTH-33-3/2009 (H1N1); A/swine/Alberta/OTH-33-7/2009 (H1N1); A/Beijing/502/2009 (H1N1); A/Firenze/10/2009 (H1N1); A/Hong Kong/2369/2009 (H1N1); A/Italy/85/2009 (H1N1); A/Santo Domingo/572N/2009 (H1N1); A/Catalonia/385/2009 (H1N1); A/Catalonia/386/2009 (H1N1); A/Catalonia/387/2009 (H1N1); A/Catalonia/390/2009 (H1N1); A/Catalonia/394/2009 (H1N1);A/Catalonia/397/2009 (H1N1); A/Catalonia/398/2009 (H1N1); A/Catalonia/399/2009 (H1N1); A/Sao Paulo/2303/2009 (H1N1); A/Akita/1/2009 (H1N1); A/Castro/JXP/2009 (H1N1); A/Fukushima/1/2009 (H1N1); A/Israel/276/2009 (H1N1); A/Israel/277/2009 (H1N1); A/Israel/70/2009 (H1N1); A/Iwate/1/2009 (H1N1); A/Iwate/2/2009 (H1N1); A/Kagoshima/1/2009 (H1N1); A/Osaka/180/2009 (H1N1); A/Puerto Montt/Bio87/2009 (H1N1); A/Sao Paulo/2303/2009 (H1N1); A/Sapporo/1/2009 (H1N1); A/Stockholm/30/2009 (H1N1); A/Stockholm/31/2009 (H1N1); A/Stockholm/32/2009 (H1N1); A/Stockholm/33/2009 (H1N1); A/Stockholm/34/2009 (H1N1); A/Stockholm/35/2009 (H1N1); A/Stockholm/36/2009 (H1N1); A/Stockholm/38/2009 (H1N1); A/Stockholm/39/2009 (H1N1); A/Stockholm/40/2009 (H1N1;) A/Stockholm/42/2009 (H1N1); A/Stockholm/43/2009 (H1N1); A/Stockholm/44/2009 (H1N1); A/Utsunomiya/2/2009 (H1N1); A/WRAIR/0573N/2009 (H1N1); and A/Zhejiang/DTID-ZJU01/2009 (H1N1).

In certain embodiments, the influenza viruses, or influenza virus polypeptides, genes or genome segments for use as described herein are obtained or derived from an influenza B virus. In certain embodiments, the influenza viruses, or influenza virus polypeptides, genes or genome segments for use as described herein are obtained or derived from a single influenza B virus subtype/lineage or strain. In other embodiments, the influenza viruses, or influenza virus polypeptides, genes or genome segments for use as described herein are obtained or derived from two or more influenza B virus subtypes or strains.

Non-limiting examples of influenza B viruses include strain Aichi/5/88, strain B/Brisbane/60/2008; Akita/27/2001, strain Akita/5/2001, strain Alaska/16/2000, strain Alaska/1777/2005, strain Argentina/69/2001, strain Arizona/146/2005, strain Arizona/148/2005, strain Bangkok/163/90, strain Bangkok/34/99, strain Bangkok/460/03, strain Bangkok/54/99, strain Barcelona/215/03, strain Beijing/15/84, strain Beijing/184/93, strain Beijing/243/97, strain Beijing/43/75, strain Beijing/5/76, strain Beijing/76/98, strain Belgium/WV106/2002, strain Belgium/WV107/2002, strain Belgium/WV109/2002, strain Belgium/WV114/2002, strain Belgium/WV122/2002, strain Bonn/43, strain Brazil/952/2001, strain Bucharest/795/03, strain Buenos Aires/161/00), strain Buenos Aires/9/95, strain Buenos Aires/SW16/97, strain Buenos Aires/VL518/99, strain Canada/464/2001, strain Canada/464/2002, strain Chaco/366/00, strain Chaco/R113/00, strain Cheju/303/03, strain Chiba/447/98, strain Chongqing/3/2000, strain clinical isolate SA1 Thailand/2002, strain clinical isolate SA10 Thailand/2002, strain clinical isolate SA100 Philippines/2002, strain clinical isolate SA101 Philippines/2002, strain clinical isolate SA110 Philippines/2002), strain clinical isolate SA112 Philippines/2002, strain clinical isolate SA113 Philippines/2002, strain clinical isolate SA114 Philippines/2002, strain clinical isolate SA2 Thailand/2002, strain clinical isolate SA20 Thailand/2002, strain clinical isolate SA38 Philippines/2002, strain clinical isolate SA39 Thailand/2002, strain clinical isolate SA99 Philippines/2002, strain CNIC/27/2001, strain Colorado/2597/2004, strain Cordoba/VA418/99, strain Czechoslovakia/16/89, strain Czechoslovakia/69/90, strain Daeku/10/97, strain Daeku/45/97, strain Daeku/47/97, strain Daeku/9/97, strain B/Du/4/78, strain B/Durban/39/98, strain Durban/43/98, strain Durban/44/98, strain B/Durban/52/98, strain Durban/55/98, strain Durban/56/98, strain England/1716/2005, strain England/2054/2005), strain England/23/04, strain Finland/154/2002, strain Finland/159/2002, strain Finland/160/2002, strain Finland/161/2002, strain Finland/162/03, strain Finland/162/2002, strain Finland/162/91, strain Finland/164/2003, strain Finland/172/91, strain Finland/173/2003, strain Finland/176/2003, strain Finland/184/91, strain Finland/188/2003, strain Finland/190/2003, strain Finland/220/2003, strain Finland/WV5/2002, strain Fujian/36/82, strain Geneva/5079/03, strain Genoa/11/02, strain Genoa/2/02, strain Genoa/21/02, strain Genova/54/02, strain Genova/55/02, strain Guangdong/05/94, strain Guangdong/08/93, strain Guangdong/5/94, strain Guangdong/55/89, strain Guangdong/8/93, strain Guangzhou/7/97, strain Guangzhou/86/92, strain Guangzhou/87/92, strain Gyeonggi/592/2005, strain Hannover/2/90, strain Harbin/07/94, strain Hawaii/10/2001, strain Hawaii/1990/2004, strain Hawaii/38/2001, strain Hawaii/9/2001, strain Hebei/19/94, strain Hebei/3/94), strain Henan/22/97, strain Hiroshima/23/2001, strain Hong Kong/110/99, strain Hong Kong/1115/2002, strain Hong Kong/112/2001, strain Hong Kong/123/2001, strain Hong Kong/1351/2002, strain Hong Kong/1434/2002, strain Hong Kong/147/99, strain Hong Kong/156/99, strain Hong Kong/157/99, strain Hong Kong/22/2001, strain Hong Kong/22/89, strain Hong Kong/336/2001, strain Hong Kong/666/2001, strain Hong Kong/9/89, strain Houston/1/91, strain Houston/1/96, strain Houston/2/96, strain Hunan/4/72, strain Ibaraki/2/85, strain ncheon/297/2005, strain India/3/89, strain India/77276/2001, strain Israel/95/03, strain Israel/WV187/2002, strain Japan/1224/2005, strain Jiangsu/10/03, strain Johannesburg/1/99, strain Johannesburg/96/01, strain Kadoma/1076/99, strain Kadoma/122/99, strain Kagoshima/15/94, strain Kansas/22992/99, strain Khazkov/224/91, strain Kobe/1/2002, strain, strain Kouchi/193/99, strain Lazio/1/02, strain Lee/40, strain Leningrad/129/91, strain Lissabon/2/90), strain Los Angeles/1/02, strain Lusaka/270/99, strain Lyon/1271/96, strain Malaysia/83077/2001, strain Maputo/1/99, strain Mar del Plata/595/99, strain Maryland/1/01, strain Memphis/1/01, strain Memphis/12/97-MA, strain Michigan/22572/99, strain Mie/1/93, strain Milano/1/01, strain Minsk/318/90, strain Moscow/3/03, strain Nagoya/20/99, strain Nanchang/1/00, strain Nashville/107/93, strain Nashville/45/91, strain Nebraska/2/01, strain Netherland/801/90, strain Netherlands/429/98, strain New York/1/2002, strain NIB/48/90, strain Ningxia/45/83, strain Norway/1/84, strain Oman/16299/2001, strain Osaka/1059/97, strain Osaka/983/97-V2, strain Oslo/1329/2002, strain Oslo/1846/2002, strain Panama/45/90, strain Paris/329/90, strain Parma/23/02, strain Perth/211/2001, strain Peru/1364/2004, strain Philippines/5072/2001, strain Pusan/270/99, strain Quebec/173/98, strain Quebec/465/98, strain Quebec/7/01, strain Roma/1/03, strain Saga/S172/99, strain Seoul/13/95, strain Seoul/37/91, strain Shangdong/7/97, strain Shanghai/361/2002), strain Shiga/T30/98, strain Sichuan/379/99, strain Singapore/222/79, strain Spain/WV27/2002, strain Stockholm/10/90, strain Switzerland/5441/90, strain Taiwan/0409/00, strain Taiwan/0722/02, strain Taiwan/97271/2001, strain Tehran/80/02, strain Tokyo/6/98, strain Trieste/28/02, strain Ulan Ude/4/02, strain United Kingdom/34304/99, strain USSR/100/83, strain Victoria/103/89, strain Vienna/1/99, strain Wuhan/356/2000, strain WV194/2002, strain Xuanwu/23/82, strain Yamagata/1311/2003, strain Yamagata/K500/2001, strain Alaska/12/96, strain GA/86, strain NAGASAKI/1/87, strain Tokyo/942/96, strain B/Wisconsin/1/2010; and strain Rochester/02/2001. In a specific embodiment, an influenza B virus is influenza a B/Colorado/06/2017-like virus (B/Victoria/2/87 lineage), influenza B/Phuket/3073/2013-like virus (B/Yamagata/16/88 lineage), influenza B/Washington/02/2019-like (B/Victoria lineage) virus, or influenza B/Phuket/3073/2013-like (B/Yamagata lineage) virus.

Other examples of influenza viruses may be found elsewhere in the application, such as in, e.g., Section 6 below. In a specific embodiment, a seasonal influenza virus strain may be used.

In certain embodiments, the influenza viruses provided herein have an attenuated phenotype. In specific embodiments, the attenuated influenza virus is based on influenza A virus. In specific embodiments, the attenuated influenza virus comprises, encodes, or both, a recombinant neuraminidase and has a backbone of an influenza A virus. In some embodiments, the attenuated influenza virus is based on influenza B virus. In specific embodiments, the attenuated influenza virus comprises, encodes, or both, a recombinant neuraminidase and has a backbone of an influenza B virus.

In specific embodiments, attenuation of influenza virus is desired such that the virus remains, at least partially, infectious and can replicate in vivo, but only generate low titers resulting in subclinical levels of infection that are non-pathogenic. Such attenuated viruses are especially suited for embodiments described herein wherein the virus or an immunogenic composition thereof is administered to a subject to induce an immune response. Attenuation of the influenza virus can be accomplished according to any method known in the art, such as, e.g., selecting viral mutants generated by chemical mutagenesis, mutation of the genome by genetic engineering, selecting reassortant viruses that contain segments with attenuated function (e.g., truncated NS1 protein (see, e.g., Hai et al., 2008, Journal of Virology 82(21):10580-10590, which is incorporated by reference herein in its entirety) or NS1 deletion (see, e.g., Wressnigg et al., 2009, Vaccine 27:2851-2857, which is incorporated by reference herein in its entirety)), or selecting for conditional virus mutants (e.g., cold-adapted viruses, see, e.g., Alexandrova et al., 1990, Vaccine, 8:61-64, which is incorporated by reference herein in its entirety). Alternatively, naturally occurring attenuated influenza viruses may be used as influenza virus backbones for the influenza virus vectors.

In a specific embodiment, the influenza A virus A/Puerto Rico/8/34 strain is used as the backbone to express a recombinant neuraminidase described herein. In another specific embodiment, the virion of the influenza A virus A/Puerto Rico/8/34 strain contains a recombinant neuraminidase described herein. In another specific embodiment, the influenza A virus A/Puerto Rico/8/34 strain is used to express a recombinant neuraminidase described herein and the virion of the A/Puerto Rico/8/34 strain contains the recombinant neuraminidase.

In a specific embodiment, an influenza A virus lacking the NS1 protein (e.g., a delNS1 virus, such as described, e.g., in U.S. Pat. No. 6,468,544; Garcia-Sastre et al., 1998, Virology 252: 324; or Mossler et al., 2013, Vaccine 31: 6194) is used as the backbone to express a recombinant neuraminidase described herein. In another specific embodiment, the virion of an influenza virus lacking the NS1 protein (e.g., a delNS1 virus, such as described, e.g., in U.S. Pat. No. 6,468,544; Garcia-Sastre et al., 1998, Virology 252: 324; or Mossler et al., 2013, Vaccine 31: 6194) contains a recombinant neuraminidase described herein. In another specific embodiment, an influenza virus lacking the NS1 protein (e.g., a delNS1 virus, such as described, e.g., in U.S. Pat. No. 6,468,544; Garcia-Sastre et al., 1998, Virology 252: 324; or Mössler et al., 2013, Vaccine 31: 6194) is used to express a recombinant neuraminidase described herein and the virion of such a virus contains the recombinant neuraminidase.

In a specific embodiment, a cold-adapted influenza A virus strain is used as the backbone to express a recombinant neuraminidase described herein. In another specific embodiment, the virion of the cold-adapted strain contains a recombinant neuraminidase described herein. In another specific embodiment, the cold-adapted influenza A virus is used to express a recombinant neuraminidase described herein and the virion of the cold-adapted influenza virus contains the recombinant neuraminidase. In one embodiment, the cold-adapted influenza A virus is A/Ann Arbor/6/60. In another embodiment, the cold-adapted influenza A virus is A/Leningrad/134/17/57. In another embodiment, a seasonal influenza virus strain is used as the backbone to express a recombinant neuraminidase described herein.

In certain embodiments, an influenza virus comprising a recombinant neuraminidase described herein has one, two, or more of the functions of an influenza virus comprising a wild-type influenza virus NA. A nonlimiting example of a function of a wild-type influenza virus NA include cleavage of sialic acid. In a specific embodiment, an influenza virus comprising a recombinant neuraminidase described herein cleaves sialic acid. Assays known to one skilled in the art can be utilized to assess the ability of a recombinant neuraminidase to cleave sialic acid.

### 5.5 Compositions

In one aspect, provided herein are compositions comprising a recombinant neuraminidase described herein. An influenza virus comprising a recombinant neuraminidase described herein may be incorporated into a composition. In a particular embodiment, an influenza virus described herein (e.g., in Section 5.4 or 6) is incorporated into a composition. In a specific embodiment, a composition is a pharmaceutical composition, such as an immunogenic composition (e.g., a vaccine formulation). The pharmaceutical compositions provided herein can be in any form that allows for the composition to be administered to a subject. In a specific embodiment, the pharmaceutical compositions are suitable for veterinary and/or human administration. The compositions may be used in methods of preventing an influenza virus disease. The compositions may be used in methods to induce an immune response against influenza virus. The compositions may be used in methods to immunize against influenza virus.

In a specific embodiment, a pharmaceutical composition (e.g., immunogenic composition) comprises a recombinant neuraminidase described herein, and optionally an adjuvant. In another specific embodiment, a pharmaceutical composition (e.g., immunogenic composition) comprises a recombinant neuraminidase described herein in an admixture with a pharmaceutically acceptable carrier. In a specific embodiment, a pharmaceutical composition (e.g., immunogenic composition) comprises an adjuvant (e.g., an adjuvant described herein) and a recombinant neuraminidase described herein, in an admixture with a pharmaceutically acceptable carrier.

In a specific embodiment, a pharmaceutical composition (e.g., immunogenic composition) comprises a nucleic acid sequence comprising a nucleotide sequence encoding a recombinant neuraminidase described herein or a vector (e.g., an expression vector) comprising a nucleic acid sequence encoding a recombinant neuraminidase described herein, and optionally an adjuvant. In another specific embodiment, a pharmaceutical composition (e.g., immunogenic composition) comprises a nucleic acid sequence comprising a nucleotide sequence encoding a recombinant neuraminidase described herein or a vector (e.g., an expression vector) comprising a nucleic acid sequence encoding a recombinant neuraminidase described herein in an admixture with a pharmaceutically acceptable carrier. In a specific embodiment, a pharmaceutical composition (e.g., immunogenic composition) comprises an adjuvant (e.g., an adjuvant described herein) and a nucleic acid sequence comprising a nucleotide sequence encoding a recombinant neuraminidase described herein or a vector (e.g., an expression vector) comprising a nucleic acid sequence encoding a recombinant neuraminidase described herein, in an admixture with a pharmaceutically acceptable carrier. In some embodiments, an immunogenic composition comprises a nucleic acid sequence or vector described herein contained in or associated with a lipid particle, nanoparticle or a liposomal particle.

In a specific embodiment, a pharmaceutical composition (e.g., immunogenic composition) comprises an influenza virus comprising a recombinant neuraminidase described herein, and optionally an adjuvant. In another specific embodiment, a pharmaceutical composition (e.g., immunogenic composition) comprises an influenza virus comprising a recombinant neuraminidase described herein in an admixture with a pharmaceutically acceptable carrier. In a specific embodiment, a pharmaceutical composition (e.g., immunogenic composition) comprises an adjuvant (e.g., an adjuvant described herein) and an influenza virus comprising a recombinant neuraminidase described herein, in an admixture with a pharmaceutically acceptable carrier.

In another specific embodiment, a pharmaceutical composition (e.g., immunogenic composition) comprises an influenza virus (e.g., a seasonal influenza virus strain) and a recombinant neuraminidase described herein in an admixture with a pharmaceutically acceptable carrier. In a specific embodiment, a pharmaceutical composition (e.g., immunogenic composition) comprises an adjuvant (e.g., an adjuvant described herein), an influenza virus (e.g., a seasonal influenza virus strain), and a recombinant neuraminidase described herein, in an admixture with a pharmaceutically acceptable carrier. The influenza virus may be live attenuated or inactivated. In addition, the composition may comprise more than one influenza virus, which is live attenuated or inactivated, and more than one recombinant neuraminidase described herein. The influenza virus composition may be trivalent or quadrivalent. In some embodiments, the composition further comprises an adjuvant.

In another specific embodiment, provided herein is trivalent inactivated influenza virus vaccine supplemented with a recombinant neuraminidase described herein. In another specific embodiment, provided herein is a quadrivalent inactivated influenza virus vaccine supplemented with a recombinant neuraminidase described herein. In another specific embodiment, provided herein is a live attenuated influenza virus vaccine supplemented with a recombinant neuraminidase described herein. In some embodiments, the composition further comprises an adjuvant.

In a specific embodiment, a pharmaceutical composition (e.g., immunogenic composition) comprises a live virus (e.g., a live attenuated virus) comprising a recombinant neuraminidase described herein, and optionally an adjuvant. In another specific embodiment, a pharmaceutical composition (e.g., immunogenic composition) comprises an inactivated virus comprising a recombinant neuraminidase described herein, and optionally an adjuvant. In another specific embodiment, a pharmaceutical composition (e.g., immunogenic composition) comprises a nucleic acid sequence comprising a nucleotide sequence encoding a recombinant neuraminidase described herein, and optionally an adjuvant. In another specific embodiment, a pharmaceutical composition (e.g., immunogenic composition) comprises a vector comprising a nucleic acid sequence encoding a recombinant neuraminidase described herein, and optionally an adjuvant.

In a specific embodiment, provided herein is a composition comprising an antibody that binds to influenza virus neuraminidase, which was generated using a recombinant neuraminidase or an influenza virus described herein.

In certain embodiments, a pharmaceutical composition (e.g., an immunogenic composition) may comprise one or more other therapies in addition to a therapy that utilizes a recombinant neuraminidase described herein described herein. In some embodiments, a pharmaceutical composition (e.g., an immunogenic composition) may comprise one or more other therapies in addition to a therapy that utilizes an influenza virus comprising a recombinant neuraminidase described herein. In certain embodiments, a pharmaceutical composition (e.g., an immunogenic composition) may comprise one or more other therapies in addition to a therapy that utilizes a nucleic acid sequence comprising a nucleotide sequence encoding a recombinant neuraminidase described herein or a vector comprising a nucleic acid sequence encoding a recombinant neuraminidase described herein.

As used herein, the term “pharmaceutically acceptable” means approved by a regulatory agency of the Federal or a state government or listed in the U.S. Pharmacopeia or other generally recognized pharmacopeiae for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, pharmaceutical compositions are formulated to be suitable for the intended route of administration to a subject. For example, the pharmaceutical composition may be formulated to be suitable for parenteral, oral, intradermal, transdermal, colorectal, intraperitoneal, and rectal administration. In a specific embodiment, the pharmaceutical composition may be formulated for intravenous, oral, intraperitoneal, intranasal, intratracheal, subcutaneous, intramuscular, topical, intradermal, transdermal or pulmonary administration. In a specific embodiment, the pharmaceutical composition may be formulated for intramuscular administration. In another specific embodiment, the pharmaceutical composition may be formulated for subcutaneous administration. In another specific embodiment, the pharmaceutical composition may be formulated for intranasal administration.

In specific embodiments, immunogenic compositions described herein are monovalent formulations. In other embodiments, immunogenic compositions described herein are multivalent formulations. In one example, a multivalent formulation comprises more than one influenza virus comprising a recombinant neuraminidase described herein.

In certain embodiments, an immunogenic composition described herein comprises more than one recombinant neuraminidase described herein. For example, an immunogenic composition may comprise 2, 3 or 4 recombinant neuraminidase described herein (e.g., a recombinant N1 and a recombinant influenza B virus NA).

An immunogenic composition described herein may be used to immunize a subject against influenza virus. An immunogenic composition described herein may also be used to prevent an influenza virus disease in a subject. In a specific embodiment, an immunogenic composition described herein may be used in a method described herein.

In certain embodiments, the pharmaceutical compositions (e.g., immunogenic compositions) described herein additionally comprise one or more components used to inactivate a virus, e.g., formalin or formaldehyde or a detergent such as sodium deoxycholate, octoxynol 9 (Triton X-100), and octoxynol 10. In other embodiments, the pharmaceutical compositions described herein do not comprise any components used to inactivate a virus.

In certain embodiments, the pharmaceutical compositions (e.g., immunogenic compositions) described herein additionally comprise one or more buffers, e.g., phosphate buffer and sucrose phosphate glutamate buffer. In other embodiments, the pharmaceutical compositions described herein do not comprise buffers.

The pharmaceutical compositions (e.g., immunogenic compositions) described herein can be included in a container, pack, or dispenser together with instructions for administration.

The pharmaceutical compositions (e.g., immunogenic compositions) described herein can be stored before use, e.g., the pharmaceutical compositions can be stored frozen (e.g., at about −20° C. or at about −70° C.); stored in refrigerated conditions (e.g., at about 4° C.); or stored at room temperature (see International Application No. PCT/IB2007/001149 published as International Publication No. WO 07/110776, which is herein incorporated by reference in its entirety, for methods of storing compositions comprising influenza vaccines without refrigeration).

In a specific embodiment, an immunogenic composition is an inactivated vaccine comprising an adjuvant (e.g., an adjuvant described in Section 5.5.3 below) and a recombinant neuraminidase (NA). The inactivated vaccine may be a whole virus inactivated vaccine or split virion vaccine. Techniques for producing such vaccines are known to one of skill in the art. In a specific embodiment, an immunogenic composition comprises formalin-inactivated whole virus particles for vaccination through the intramuscular route.

### 5.5.1 Live Virus Vaccines

In one embodiment, provided herein are immunogenic compositions (e.g., vaccines) comprising live virus containing a recombinant neuraminidase described herein. In another embodiment, provided herein are immunogenic compositions (e.g., vaccines) comprising live virus that is engineered to encode a recombinant neuraminidase described herein, which is expressed by progeny virus produced in the subjects administered the compositions. In specific embodiments, the recombinant neuraminidase is membrane-bound. In other specific embodiments, the recombinant neuraminidase is not membrane-bound, i.e., it is soluble. In particular embodiments, the live virus is an influenza virus, such as described in Section 5.4. In some embodiments, the live virus is attenuated. In a specific embodiment, the live virus is a live attenuated influenza virus.

In a specific embodiment, the live virus that contains a recombinant neuraminidase is propagated in embryonated chicken eggs before its use in an immunogenic composition described herein. In another specific embodiment, the live virus that contains a recombinant neuraminidase is not propagated in embryonated chicken eggs before its use in an immunogenic composition described herein. In another specific embodiment, the live virus that contains a recombinant neuraminidase is propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) before its use in an immunogenic composition described herein.

An immunogenic composition comprising a live virus for administration to a subject may be preferred because multiplication of the virus in the subject may lead to a prolonged stimulus of similar kind and magnitude to that occurring in natural infections, and therefore, confer substantial, long lasting immunity.

### 5.5.2 Inactivated Virus Vaccines

In one embodiment, provided herein are immunogenic compositions (e.g., vaccines) comprising an inactivated virus containing a recombinant neuraminidase. In specific embodiments, the recombinant neuraminidase is membrane-bound. In particular embodiments, the inactivated virus is an influenza virus, such as described in Section 5.4 or 6. In certain embodiments, the inactivated virus immunogenic compositions comprise one or more adjuvants.

Techniques known to one of skill in the art may be used to inactivate viruses containing a recombinant neuraminidase. Common methods use formalin, heat, or detergent for inactivation. See, e.g., U.S. Pat. No. 6,635,246, which is herein incorporated by reference in its entirety. Other methods include those described in U.S. Pat. Nos. 5,891,705; 5,106,619 and 4,693,981, which are incorporated herein by reference in their entireties.

In a specific embodiment, an immunogenic composition described herein is a split vaccine. Techniques for producing split virus vaccines are known to those skilled in the art. By way of non-limiting example, an influenza virus split vaccine may be prepared using inactivated particles disrupted with detergents. One example of a split virus vaccine that can be adapted for use in accordance with the methods described herein is the Fluzone®, Influenza Virus Vaccine (Zonal Purified, Subvirion) for intramuscular use, which is formulated as a sterile suspension prepared from influenza viruses propagated in embryonated chicken eggs. The virus-containing fluids are harvested and inactivated with formaldehyde. Influenza virus is concentrated and purified in a linear sucrose density gradient solution using a continuous flow centrifuge. The virus is then chemically disrupted using a nonionic surfactant, octoxinol-9, (Triton® X-100—A registered trademark of Union Carbide, Co.) producing a “split virus.” The split virus is then further purified by chemical means and suspended in sodium phosphate-buffered isotonic sodium chloride solution.

In a specific embodiment, the inactivated virus that contains a recombinant neuraminidase was propagated in embryonated chicken eggs before its inactivation and subsequent use in an immunogenic composition described herein. In another specific embodiment, the inactivated virus that contains a recombinant neuraminidase was not propagated in embryonated chicken eggs before its inactivation and subsequent use in an immunogenic composition described herein. In another specific embodiment, the inactivated virus that contains a recombinant neuraminidase was propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) before its inactivation and subsequent use in an immunogenic composition described herein.

### 5.5.3 Adjuvants

In certain embodiments, the compositions described herein comprise, or are administered in combination with, an adjuvant. The adjuvant for administration in combination with a composition described herein may be administered before, concommitantly with, or after administration of said composition. In some embodiments, the adjuvant enhance or boosts an immune response to influenza virus and does not produce an allergy or other adverse reaction. Adjuvants can enhance an immune response by several mechanisms including, e.g., lymphocyte recruitment, stimulation of B and/or T cells, and stimulation of macrophages.

In certain embodiments, an adjuvant augments the intrinsic response to a recombinant neuraminidase without causing conformational changes in the polypeptide that affect the qualitative form of the response. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as polyglutamic acid or polylysine, or other immunopotentiating agents. An adjuvant may be AddaVax. Another adjuvant is one described in Section 6.1 or 6.2, infra.

### 5.6 Prophylactic and Therapeutic Uses

In one aspect, provided herein are methods for inducing an immune response in a subject utilizing a recombinant neuraminidase described herein or a composition thereof. In a specific embodiment, a method for inducing an immune response to an influenza virus in a subject comprises administering to a subject in need thereof an effective amount of an immunogenic composition described herein.

In another aspect, provided herein are methods for inducing an immune response in a subject utilizing a nucleic acid sequence comprising a nucleotide sequence encoding a recombinant neuraminidase described herein, or a composition thereof. In a specific embodiment, a method for inducing an immune response to an influenza virus in a subject comprises administering to a subject in need thereof an effective amount of a composition comprising a nucleic acid sequence comprising a nucleotide sequence encoding a recombinant neuraminidase described herein. In another specific embodiment, a method for inducing an immune response to an influenza virus in a subject comprises administering to a subject in need thereof an effective amount of a composition comprising a vector that comprises a nucleic acid sequence encoding a recombinant neuraminidase described herein.

In another aspect, provided herein are methods for inducing an immune response in a subject utilizing an influenza virus containing, engineered to express a recombinant neuraminidase described herein, or both, or a composition described herein. In a specific embodiment, a method for inducing an immune response to an influenza virus in a subject comprises administering to a subject in need thereof an effective amount of an influenza virus containing, engineered to express a recombinant neuraminidase described herein, or both, or an immunogenic composition thereof.

In a specific embodiment, a method for inducing an immune response to an influenza virus in a subject comprises administering to a subject in need thereof a live virus vaccine described herein. In particular embodiments, the live virus vaccine comprises an attenuated virus. In another embodiment, a method for inducing an immune response to an influenza virus in a subject comprises administering to a subject in need thereof an inactivated virus vaccine described herein. In another embodiment, a method for inducing an immune response to an influenza virus in a subject comprises administering to a subject in need thereof a split virus vaccine described herein.

In another aspect, provided herein are methods for immunizing against influenza virus in a subject utilizing a recombinant neuraminidase described herein or a composition thereof. In a specific embodiment, a method for immunizing against influenza virus comprises administering to a subject in need thereof an effective amount of an immunogenic composition described herein.

In one aspect, provided herein are methods for immunizing against influenza virus in a subject utilizing a nucleic acid sequence comprising a nucleotide sequence encoding a recombinant neuraminidase described herein, or a composition thereof. In a specific embodiment, a method for immunizing against influenza virus comprises administering to a subject in need thereof an effective amount of a composition comprising a nucleic acid sequence comprising a nucleotide sequence encoding a recombinant neuraminidase described herein. In another specific embodiment, a method for immunizing against influenza virus in a subject comprises administering to a subject in need thereof an effective amount of a composition comprising a vector (e.g., an expression vector) that comprises a nucleic acid sequence encoding a recombinant neuraminidase described herein.

In another aspect, provided herein are methods for immunizing against influenza virus in a subject utilizing an influenza virus containing, engineered to express a recombinant neuraminidase described herein, or both, or a composition described herein. In a specific embodiment, a method for immunizing against influenza virus comprises administering to a subject in need thereof an effective amount of an influenza virus containing, engineered to express a recombinant neuraminidase described herein, or both, or an immunogenic composition thereof.

In a specific embodiment, a method for immunizing against influenza virus comprises administering to a subject in need thereof a live virus vaccine described herein. In particular embodiments, the live virus vaccine comprises an attenuated virus. In another embodiment, a method for immunizing against influenza virus comprises administering to a subject in need thereof an inactivated virus vaccine described herein. In another embodiment, a method for immunizing against influenza virus in a subject comprises administering to a subject in need thereof a split virus vaccine described herein.

In another aspect, provided herein is a method for immunizing against influenza virus, comprising administering to the subject an immunogenic composition described herein (e.g., in Section 5.5 above) and administering to the subject an adjuvant described herein. In one embodiment, provided herein is a method for immunizing against influenza virus in a subject, comprising administering to the subject an immunogenic composition described herein (e.g., in Section 5.5 above) in combination with an adjuvant described herein. The immunogenic composition may be administered to the subject concurrently with, prior to (e.g., less than 5 minutes, less than 10 minutes, less than 15 minutes, less than 30 minutes, less than 45 minutes, less than 60 minutes, less than 1.5 hours, or less than 2 hours prior to), or subsequent to (e.g., less than 5 minutes, less than 10 minutes, less than 15 minutes, less than 30 minutes, less than 45 minutes, less than 60 minutes, less than 1.5 hours, or less than 2 hours after) the administration of an adjuvant described herein. In a specific embodiment, the immunogenic composition and the adjuvant described herein are administered via the same route of administration. In other embodiments, the immunogenic composition and the adjuvant are administered via different routes of administration. In a specific embodiment, the immunogenic composition comprises an inactivated influenza virus containing a recombinant NA described herein. In another specific embodiment, the immunogenic composition comprises a split influenza virus, wherein the split influenza virus comprises a recombinant NA described herein. In some embodiments, the immunogenic composition does not comprise an adjuvant.

In another embodiment, provided herein are immunization regimens involving a first immunization (e.g., priming) with an immunogenic composition (e.g., a vaccine) described herein followed by one, two, or more additional immunizations (e.g., boostings) with an immunogenic composition (e.g., a vaccine). In one embodiment, an immunogenic regimen involves a first immunization (e.g., priming) with an immunogenic composition (e.g., a vaccine) described herein followed by one additional immunizations (e.g., boost) with an immunogenic composition (e.g., a vaccine). In a specific embodiment, the immunogenic composition (e.g., a vaccine) used in the first immunization is the same type of an immunogenic composition (e.g., a vaccine) used in one, two or more additional immunizations. For example, if the immunogenic composition (e.g., vaccine) used in the first immunization is an inactivated influenza virus vaccine formulation, the immunogenic composition (e.g., vaccine) used for the one, two or more additional immunizations may be the same type of vaccine formulation, i.e., an inactivated influenza virus vaccine formulation. In other specific embodiments, the immunogenic composition (e.g., vaccine) used in the first immunization is different from the type of immunogenic composition (e.g., vaccine) used in one, two or more additional immunizations. For example, if the immunogenic composition (e.g., vaccine) used in the first immunization is a live influenza virus vaccine formulation, the immunogenic composition (e.g., vaccine) used in the one, two or more additional immunizations is another type of vaccine formulation, such as an inactivated influenza virus. In another example, if the immunogenic composition (e.g., vaccine) used in the first immunization is a live attenuated influenza virus vaccine formulation, the immunogenic composition (e.g., vaccine) used in the one, two or more additional immunizations is another type of vaccine formulation, such as an inactivated influenza virus. In certain embodiments, the vaccine formulation used in the additional immunizations changes. For example, if a live attenuated influenza virus vaccine formulation is used for one additional immunization, then one or more additional immunizations may use a different vaccine formulation, such as an inactivated vaccine formulation. In a particular embodiment, a live influenza virus vaccine formulation is administered to a subject followed by an inactivated vaccine formulation (e.g., split virus vaccine or subunit vaccine). In a specific embodiment, an immunization regimen is analogous to the regimen described in Section 6, infra (e.g., Example 1 or 3, infra).

In a specific embodiment, in accordance with the methods described herein a subject may be administered one, two or more doses of an immunogenic composition described herein.

In a specific embodiment, a subject is immunized in accordance with a method described herein prior, during or both flu season. In a specific embodiment, flu season in the U.S. may be from September or October of one year through March or April of the next year.

In some embodiments, the immune response induced by an immunogenic composition described herein is effective to prevent an influenza virus disease caused by one, two, or more subtypes of influenza A virus. In some embodiments, the immune response induced by an immunogenic composition described herein is effective to prevent an influenza virus disease caused by one, two, three or more strains of influenza virus. In certain embodiments, the immune response induced by an immunogenic composition described herein is effective to prevent an influenza virus disease caused by a subtype of influenza virus that belongs to one NA group and not another NA group. In some embodiments, the immune response induced by an immunogenic composition described herein is effective to prevent an influenza virus disease caused by one or more strains within the same subtype of influenza A virus. In certain embodiments, the immune response induced by an immunogenic composition described herein is effective to prevent an influenza virus disease caused by one, two, three or more strains within the same subtype of influenza A virus.

In some embodiments, the immune response induced by an immunogenic composition described herein is effective to reduce the number of symptoms resulting from an influenza virus disease/infection. In certain embodiments, the immune response induced by an immunogenic composition described herein is effective to reduce the duration of one or more symptoms resulting from an influenza virus disease/infection. Symptoms of influenza virus disease/infection include, but are not limited to, body aches (especially joints and throat), fever, nausea, headaches, irritated eyes, fatigue, sore throat, reddened eyes or skin, and abdominal pain.

In some embodiments, the immune response induced by an immunogenic composition described herein is effective to reduce the hospitalization of a subject suffering from an influenza virus disease/infection. In some embodiments, the immune response induced by an immunogenic composition described herein is effective to reduce the duration of hospitalization of a subject suffering from an influenza virus disease/infection.

In a specific embodiment, the immune response induced by an immunogenic composition described herein induces NA-specific antibodies (e.g., IgG). In another specific embodiment, the immune response induced by an immunogenic composition described herein induces antibodies with ADCC activity as assessed by a technique known to one of skill in the art or described herein. In another specific embodiment, the immune response induced by an immunogenic composition described herein induces antibodies with neuraminidase inhibition activity as assessed by a technique known to one of skill in the art or described herein. In another specific embodiment, the immune response induced by an immunogenic composition described herein induces antibodies with (1) ADCC activity as assessed by a technique known to one of skill in the art; and (2) neuraminidase inhibition activity as assessed by a technique known to one of skill in the art or described herein.

In another aspect, provided herein are methods for preventing an influenza virus disease in a subject utilizing an immunogenic composition described herein. In a specific embodiment, a method for preventing an influenza virus disease in a subject comprises administering to a subject in need thereof a recombinant neuraminidase described herein. In a specific embodiment, a method for preventing an influenza virus disease in a subject comprises administering to a subject in need thereof a nucleic acid sequence comprising a nucleotide sequence encoding a recombinant neuraminidase, or a vector comprising a nucleic acid sequence encoding a recombinant neuraminidase. In a specific embodiment, a method for preventing an influenza virus disease in a subject comprises administering to a subject in need thereof a live virus vaccine, an inactivated virus vaccine, or a split virus vaccine described herein. In a specific embodiment, a method for preventing an influenza virus disease in a subject comprises administering to a subject in need thereof a live virus vaccine described herein. In particular embodiments, the live virus vaccine comprises an attenuated virus. In another embodiment, a method for preventing an influenza virus disease in a subject comprises administering to a subject in need thereof an inactivated virus vaccine described herein. In another embodiment, a method for preventing or an influenza virus disease in a subject comprises administering to a subject in need thereof a split virus vaccine described herein. In another embodiment, a method for preventing an influenza virus disease in a subject comprises administering to a subject in need thereof an immunogenic composition described herein.

In another aspect, provided herein are methods for preventing an influenza virus disease, or treating an influenza virus infection or an influenza virus disease in a subject comprising administering to a subject an anti-influenza virus NA antibody(ies), wherein the anti-influenza virus NA antibody(ies) was generated utilizing an immunogenic composition described herein. For example, an immunogenic composition described herein may be administered to a non-human subject (e.g., a non-human subject that expresses or is capable of expression human antibody) to generate anti-influenza virus NA antibody(ies). In a specific embodiment, provided herein is a method for preventing an influenza virus disease in a human subject comprising administering the subject a human or humanized anti-influenza virus NA antibody(ies), wherein the anti-influenza virus NA antibody(ies) was generated utilizing an immunogenic composition described herein.

In certain embodiments, the methods for preventing an influenza virus disease, or treating an influenza virus infection or an influenza virus disease in a subject (e.g., a human or non-human animal) provided herein result in a reduction in the replication of the influenza virus in the subject as measured by in vivo and in vitro assays known to those of skill in the art and described herein. In some embodiments, the replication of the influenza virus is reduced by approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. In specific embodiments, the methods for preventing an influenza virus disease, or treating an influenza virus infection or an influenza virus disease in a subject (e.g., a human or non-human animal) provided herein result in a reduction of the titer of an influenza virus detected in the subject. In specific embodiments, the methods for preventing an influenza virus disease, or treating an influenza virus infection or an influenza virus disease in a subject results in one, two, or more of the following: (1) reduces the number of symptoms of the infection/disease, (2) reduces the severity of the symptoms of the infection/disease, (3) reduces the length of the infection/disease, (4) reduces hospitalization or complications resulting from the infection/disease, (5) reduces the length of hospitalization of the subject, (6) reduces organ failure associated with the influenza virus infection/disease, and (7) increases survival of the subject. In a specific embodiment, the methods for preventing an influenza virus disease, or treating an influenza virus infection or an influenza virus disease in a subject inhibits the development or onset of an influenza virus disease or one or more symptoms thereof.

In certain embodiments, provided herein are methods for generating antibodies comprising administering an influenza virus containing, engineered to express a recombinant neuraminidase described herein, or both, or composition described herein may be administered to a subject (e.g., a non-human subject). In some embodiments, an influenza virus containing, engineered to express a recombinant neuraminidase described herein, or both, or composition described herein may be administered to a subject (e.g., a non-human subject) and the antibodies may be isolated. The isolated antibodies may be cloned. The antibodies may be humanized and/or optimized. In some embodiments, hybridomas are produced which produce a particular antibody of interest. In certain embodiments, the non-human subject administered a recombinant neuraminidase described herein or a composition described herein is capable of producing human antibodies. Techniques for isolating, cloning, humanizing, optimizing and for generating hybridomas are known to one of skill in the art. In a specific embodiment, antibodies generated by a method described herein may be utilized in assays (e.g., assays described herein) as well as in passive immunization of a subject (e.g., a human subject). Thus, provided herein, in certain embodiments, are methods for treating influenza virus infection or preventing influenza virus disease, comprising administering antibodies generated by a method described herein.

In another aspect, a recombinant neuraminidase described herein may be used to assess antibodies directed to influenza virus neuraminidase in assays, such as immunoassays.

### 5.6.1 Combination Therapies

In various embodiments, (1) a recombinant neuraminidase described herein, (2) a nucleic acid sequence comprising a nucleotide sequence encoding a recombinant neuraminidase described herein, (3) a vector comprising a nucleic acid sequence encoding a recombinant neuraminidase, or (4) an influenza virus containing, engineered to express a recombinant neuraminidase described herein, or both may be administered to a subject in combination with one or more other therapies (e.g., an antiviral, antibacterial, or immunomodulatory therapies). In some embodiments, a pharmaceutical composition (e.g., an immunogenic composition) described herein may be administered to a subject in combination with one or more therapies (e.g., an antiviral, antibacterial, or immunomodulatory therapies). The one or more other therapies may be beneficial in the prevention of an influenza virus disease or may ameliorate a symptom or condition associated with an influenza virus disease. In some embodiments, the one or more other therapies are pain relievers, anti-fever medications, or therapies that alleviate or assist with breathing. In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In specific embodiments, two or more therapies are administered within the same patient visit.

### 5.6.2 Patient Populations

In certain embodiments, a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein may be administered to a naïve subject, i.e., a subject that does not have a disease caused by influenza virus infection or has not been and is not currently infected with an influenza virus infection. In one embodiment, a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is administered to a naïve subject that is at risk of acquiring an influenza virus infection.

In another embodiment, a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is administered to a subject that does not have a disease caused by the specific influenza virus, or has not been and is not infected with the specific influenza virus to which the recombinant neuraminidase induces an immune response.

In another embodiment, a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein may also be administered to a subject that is, has been, or is and has been infected with the influenza virus or another type, subtype/lineage or strain of the influenza virus to which the recombinant neuraminidase induces an immune response.

In certain embodiments, a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is administered to a patient who has been diagnosed with an influenza virus infection. In some embodiments, a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is administered to a patient infected with an influenza virus before symptoms manifest or symptoms become severe (e.g., before the patient requires hospitalization).

In some embodiments, a subject administered a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is an animal. In certain embodiments, the animal is a bird. In certain embodiments, the animal is a canine. In certain embodiments, the animal is a feline. In certain embodiments, the animal is a horse. In certain embodiments, the animal is a cow. In certain embodiments, the animal is a mammal, e.g., a horse, swine, mouse, or primate, preferably a human.

In specific embodiments, a subject administered a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is a human infant. As used herein, the term “human infant” refers to a newborn to 1 year old human.

In specific embodiments, a subject administered a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or composition described herein is a human child. As used herein, the term “human child” refers to a human that is 1 year to 18 years old.

In specific embodiments, a subject administered a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is a human adult. As used herein, the term “human adult” refers to a human that is 18 years or older.

In specific embodiments, a subject administered a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein herein is an elderly human. As used herein, the term “elderly human” refers to a human 65 years or older.

In some embodiments, the human subject to be administered a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is any individual at increased risk of influenza virus infection or disease resulting from influenza virus infection (e.g., an immunocompromised or immunodeficient individual).

In some embodiments, the human subject to be administered a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is any individual in close contact with an individual with increased risk of influenza virus infection or disease resulting from influenza virus infection (e.g., immunocompromised or immunosuppressed individuals).

In some embodiments, the human subject to be administered a recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is an individual affected by any condition that increases susceptibility to influenza virus infection or complications or disease resulting from influenza virus infection. In other embodiments, recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein is administered to a subject in whom an influenza virus infection has the potential to increase complications of another condition that the individual is affected by, or for which they are at risk.

### 5.6.3 Routes of Delivery

A recombinant neuraminidase; a nucleic acid sequence comprising a nucleotide sequence encoding a recombinant neuraminidase; a vector comprising a nucleic acid sequence encoding a recombinant neuraminidase; an influenza virus, which contains or is engineered to express a recombinant neuraminidase described herein, or both; or a composition described herein may be delivered to a subject by a variety of routes. These include, but are not limited to, intranasal, pulmonary, intratracheal, oral, intradermal, intramuscular, intraperitoneal, transdermal, intravenous, conjunctival and subcutaneous routes as well as other routes described herein. In some embodiments, a composition is formulated for topical administration, for example, for application to the skin. In specific embodiments, the route of administration is nasal, e.g., as part of a nasal spray. In certain embodiments, a composition is formulated for intramuscular administration. In some embodiments, a composition is formulated for subcutaneous administration. In specific embodiments for live virus vaccines, the vaccine is formulated for administration by a route other than injection.

In one embodiment, a live attenuated influenza virus vaccine is administered intranasally. In another embodiment, an inactivated influenza virus vaccine (e.g., an inactivated whole virus vaccine or a split influenza virus vaccine) is administered intramuscularly.

### 5.6.4 Dosage

The amount of a recombinant neuraminidase; a nucleic acid sequence comprising a nucleotide sequence encoding a recombinant neuraminidase; a vector (e.g., an expression vector) comprising a nucleic acid sequence encoding a recombinant neuraminidase; an influenza virus, which contains or is engineered to express a recombinant neuraminidase described herein, or both; or a composition described herein, which will be effective in the prevention of an influenza virus disease will depend on the nature of the disease, and can be determined by standard clinical techniques.

The precise dose to be employed in the formulation will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, health), whether the patient is human or an animal, other medications administered, and whether treatment is prophylactic or therapeutic. Usually, the patient is a human but non-human mammals including transgenic mammals can also be treated. Treatment dosages are optimally titrated to optimize safety and efficacy.

As used herein, the term “effective amount” in the context of administering a therapy to a subject refers to the amount of a therapy which may have a prophylactic effect(s), therapeutic effect(s), or both a prophylactic and therapeutic effect(s). In certain embodiments, an “effective amount” in the context of administration of a therapy to a subject refers to the amount of a therapy which is sufficient to achieve one, two, three, four, or more of the following effects: (i) reduce or ameliorate the severity of an influenza virus infection, disease or symptom associated therewith; (ii) reduce the duration of an influenza virus infection, disease or symptom associated therewith; (iii) prevent the progression of an influenza virus infection, disease or symptom associated therewith; (iv) cause regression of an influenza virus infection, disease or symptom associated therewith; (v) prevent the development or onset of an influenza virus infection, disease or symptom associated therewith; (vi) prevent the recurrence of an influenza virus infection, disease or symptom associated therewith; (vii) reduce or prevent the spread of an influenza virus from one cell to another cell, one tissue to another tissue, or one organ to another organ; (viii) prevent or reduce the spread of an influenza virus from one subject to another subject; (ix) reduce organ failure associated with an influenza virus infection; (x) reduce hospitalization of a subject; (xi) reduce hospitalization length; (xii) increase the survival of a subject with an influenza virus infection or disease associated therewith; (xiii) eliminate an influenza virus infection or disease associated therewith; (xiv) inhibit or reduce influenza virus replication; (xv) inhibit or reduce the entry of an influenza virus into a host cell(s); (xvi) inhibit or reduce replication of the influenza virus genome; (xvii) inhibit or reduce synthesis of influenza virus proteins; (xviii) inhibit or reduce assembly of influenza virus particles; (xix) inhibit or reduce release of influenza virus particles from a host cell(s); (xx) reduce influenza virus titer; and/or (xxi) enhance or improve the prophylactic or therapeutic effect(s) of another therapy.

In certain embodiments, the effective amount does not result in complete protection from an influenza virus disease, but results in a lower titer or reduced number of influenza viruses compared to an untreated subject with an influenza virus infection. In certain embodiments, the effective amount results in a 0.5 fold, 1 fold, 1.5 fold, 2 fold, 3 fold, 4 fold, 5 fold, 6 fold, 7 fold, 8 fold, 9 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titer of influenza virus relative to an untreated subject with an influenza virus infection. In some embodiments, the effective amount results in a reduction in titer of influenza virus relative to an untreated subject with an influenza virus infection of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. Benefits of a reduction in the titer, number or total burden of influenza virus include, but are not limited to, less severe symptoms of the infection, fewer symptoms of the infection and a reduction in the length of the disease associated with the infection.

In certain embodiments, an effective amount of a therapy (e.g., a composition thereof) results in an anti-influenza virus NA titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-influenza virus NA titer in a blood sample from the subject prior to immunization. In certain embodiments, an effective amount of a therapy (e.g., a composition thereof) results in an anti-influenza virus NA stalk titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-influenza virus NA stalk titer in a blood sample from the subject prior to immunization.

In certain embodiments, a dose of an influenza virus described herein may be 104 plaque forming units (PFU) to 108 PFU. In some embodiments, a dose of a recombinant neuraminidase may contain 5 to 15 μg of the recombinant neuraminidase described herein. In certain embodiments, an inactivated vaccine is formulated such that it contains 5 to 15 μg of hemagglutinin (HA) polypeptide described herein. In some embodiments, a dose of a nucleic acid sequence or vector described herein may contain 25 micrograms to 1 milligram of the nucleic acid sequence or vector.

### 5.7 Biological Assays

Also provided herein are biological assays that may be used to characterize a recombinant NA, and viruses containing, expressing, or both such mutated influenza virus NA polypeptide. See, also, Section 6. In a specific embodiment, an assay described in Section 6 is used to characterize a recombinant NA or virus containing, expressing, or both such a recombinant NA. In another specific embodiment, an assay described in Section 6 is used to characterize the neuraminidase inhibition activity of antibodies induced by an immunogenic composition described herein. In another specific embodiment, the immunogenicity or effectiveness of an immunogenic composition described herein is assessed using one, two, or more assays described in Section 6.

### 5.7.1 Assays for Testing Activity of Recombinant Neuraminidase

Assays for testing the expression of a recombinant neuraminidase disclosed herein may be conducted using any assay known in the art. For example, an immunoassay, such as a Western blot, may be used to assess the expression of a recombinant neuraminidases. An assay for incorporation of recombinant neuraminidase into a viral vector may comprise growing a virus as described herein, purifying the viral particles by centrifugation through a sucrose cushion, and subsequent analysis for the recombinant neuraminidase expression by an immunoassay, such as Western blotting, using methods well known in the art.

In another embodiment, a recombinant neuraminidase disclosed herein is assayed for proper folding by determination of the structure or conformation of the recombinant neuraminidase using any method known in the art such as, e.g., NMR, X-ray crystallographic methods, or secondary structure prediction methods, e.g., circular dichroism.

In another embodiment, a recombinant neuraminidase disclosed herein is tested for the ability to form tetramers using a technique known in the art or described herein (e.g., in Section 6, infra). In another embodiment, a recombinant neuraminidase disclosed herein or a virus containing or expressing a recombinant neuraminidase disclosed herein is assessed for influenza virus neuraminidase activity using a technique known to one of skill in the art or described herein (e.g., in Section 6, infra).

### 5.8 Kits

In one aspect, provided herein is a pharmaceutical pack or kit for immunizing against an influenza virus in a subject comprising one or more containers filled with one or more of the ingredients of a pharmaceutical composition described herein (e.g., immunogenic compositions described herein), such as an influenza virus (e.g., a live attenuated influenza virus or an inactivated virus), or a recombinant neuraminidase, or a nucleic acid sequence comprising a nucleotide sequence encoding a recombinant neuraminidase, or a vector comprising a nucleic acid sequence encoding a recombinant neuraminidase. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in accordance with the methods described herein. In one embodiment, a kit comprises a recombinant neuraminidase described herein (such as described in Section 5.1 above or Section 6), in one or more containers. In another embodiment, a kit comprises an influenza virus described herein containing a recombinant neuraminidase (such as described in Section 5.4 above or Section 6), in one or more containers. In another embodiment, a kit comprises a nucleic acid sequence comprising a nucleotide sequence encoding a recombinant neuraminidase, in one or more containers. In another embodiment, a kit comprises a vector comprising a nucleic acid sequence encoding a recombinant neuraminidase, in one or more containers. In another embodiment, a kit comprises one or more immunogenic compositions described herein in one or more containers. In certain embodiments, a kit comprises a vaccine described herein, e.g., an inactivated influenza virus vaccine or a live influenza virus vaccine, wherein said vaccine comprises a recombinant neuraminidase described herein and optionally, an adjuvant described herein (e.g., in Section 5.5.3 or Section 6).

In certain embodiments, a kit described herein comprises: (a) a first container comprising an immunogenic composition described herein (e.g., described in Section 5.5 or Section 6); and (b) a second container comprising an adjuvant described herein (e.g., in Section 5.5.3). In specific embodiments, the immunogenic composition is an inactivated whole virus vaccine. In specific embodiments, the immunogenic composition is a split virus vaccine. In specific embodiment, the immunogenic composition is a live attenuated virus vaccine.

In certain embodiments, a kit described herein comprises: (a) a first container comprising an immunogenic composition comprising a recombinant neuraminidase described herein; and (b) a second container comprising an inactivated trivalent influenza virus vaccine, an inactivated quadravalent influenza virus vaccine or a live attenuated influenza virus vaccine. In some embodiments, the immunogenic composition further comprises an adjuvant.

### 6. Example 1: Use of Natural Tetramerization Domains and Cysteine Mutants to Produce Tetrameric Recombinant Neuraminidase-Based Vaccine Candidates

### 6.1.1 Introduction

Trivalent and quadrivalent inactivated influenza virus vaccines (TIV, QIV) induce narrow, strain specific immune responses that target the immunodominant head domain of the viral hemagglutinin (HA). Current vaccines are not efficient at inducing an immune response to the influenza virus NA (1, 2). The reasons for the lack of NA immunogenicity in TIV and QIV are not completely understood, but three major factors may contribute: 1) Low/non-standardized amounts of NA in the vaccine (1), 2) NA in TIV and QIV might not display conserved conformational epitopes correctly due to denaturation during the vaccine production process, and 3) NA is immunosubdominant to HA, especially when attached physically to HA (e.g. via the hydrophobic HA and NA transmembrane domains) (3, 4). A potential solution to this problem is to supplement the current TIV or QIV (or the recombinant influenza vaccine—RIV4) with correctly folded, stable, tetrameric recombinant protein (see FIG. 5 of reference (5)). Two ways exist to produce recombinant NA: i) recombinant expression of full length NA followed by membrane extraction and ii) expression of a soluble version of the NA followed by purification from the cell supernatant. Recombinant expression of the full length NA in cells followed by membrane extraction (6), unfortunately, results in relatively low yields and the hypervariable stalk of the NA might lead to instability over time. Expression of the head domain only (which houses the protective epitopes) is a good alternative. However, if only the NA head is expressed, no tetramer is formed, and the resulting antigen is not protective. To form a stable, functional and correctly folded tetramer, a tetramerization domain needs to be attached to the NA head domain. This has had been attempted with variable success in the past. To date, a GCN4 leucine zipper, a bacterial tetrabrachion tetramerization domain and the human vasodilator stimulated phosphoprotein (VASP) tetramerization domain have been used, with the latter providing excellent results in vaccine studies (1). However, the GNC4 leucine zipper construct does not express well and has homologies to human proteins and the VASP domain is derived from a human protein raising concerns about autoimmunity. As such, these constructs may be incompatible with GMP production and human clinical trials.

This example describes the production of a stable, immunogenic recombinant neuraminidase comprising a globular head domain of an influenza virus neuraminidase and a tetramerization. This example demonstrates that vaccination with such a stable, immunogenic recombinant neuraminiase confers complete protection against a stringent 10×LD₅₀ challenge.

In addition, this example describes the construction of cysteine mutants to form natural trimers without trimerization domains. When NA is expressed with its stalk domain it forms dimers via a disulfide bridge between two monomers. When the NA is naturally anchored in a lipid membrane, this then leads to tetramer formation (12). However, this is not the case when NA is expressed as recombinant protein (without the transmembrane domain). In this case only dimers are formed. By mutating in additional cysteines, additional disulfide bonds may be formed that then may connect two dimers to form a tetramer.

### 6.1.2 Materials and Methods

### Design and Generation of Recombinant Neuraminidase Constructs

The measles virus phosphoprotein tetramerization domain (MPP) (7), the Sendai virus phosphoprotein tetramerization domain (SPP) (8), a tetramerization domain from SEPPALLATA-like MADS domain transcription factor from Arabidopsis thaliana (SMDTF) (9), a PiLZ structure from Xhantomonas campestris (10) and Dictyocaulus viviparus ACE tetramerization domain (11) N1 fusion proteins were generated by cloning the respective codon optimized tetramerization domains into a pFastBacDual expression vector. The vector contains the N-terminal signal peptide sequence, a hexa-histidine purification tag as well as a thrombin cleavage site. Then, the globular head domain of A/Michigan/45/15 N1 was directly inserted behind the tetramerization domain.

The following sequences were used:
Italicized and bold: signal peptide and his tag
Underlined and bold: respective tetramerization domains
Bold: N1 NA
Double underlined and bold: thrombin cleavage site plus two amino acid residues

| >SMDTF-N1 | |
|---|---|
| (SEQ ID NO: 24) | |
| MLLVNQSHQGFNKEHTSKMVSAIVLYVLLAAAAHSAFAADPHHHHHHVEL | 50 |
| SSQQEYLKLKERYDALQRTQRNLLGEDLGPLSTKELESLERQLDSSLKQI | 100 |
| RALRTQFMLDQSKERMLTETNKTLRLRLADGYSLVPRGSPSRSVKLAGNS | 150 |
| SLCPVSGWAIYSKDNSVRIGSKGDVFVIREPFISCSPLECRTFFLTQGAL | 200 |
| LNDKHSNGTIKDRSPYRTLMSCPIGEVPSPYNSRFESVAWSASACHDGIN | 250 |
| WLTIGISGPDSGAVAVLKYNGIITDTIKSWRNNILRTQESECACVNGSCF | 300 |
| TIMTDGPSDGQASYKIFRIEKGKIIKSVEMKAPNYHYEECSCYPDSSEIT | 350 |
| CVCRDNWHGSNRPWVSFNQNLEYQMGYICSGVFGDNPRPNDKTGSCGPVS | 400 |
| SNGANGVKGFSFKYGNGVWIGRTKSISSRKGFEMIWDPNGWTGTDNKFSI | 450 |
| KQDIVGINEWSGYSGSFVQHPELTGLDCIRPCFWVELIRGRPEENTIWTS | 500 |
| GSSISFCGVNSDTVGWSWPDGAELPFTIDK* | |
| >SPP-N1 | |
| (SEQ ID NO: 25) | |
| MLLVNQSHQGFNKEHTSKMVSAIVLYVLLAAAAHSAFAADPHHHHHHENT | 50 |
| SSMKEMATLLTSLGVIQSAQEFESSRDASYVFARRALKSANYAEMTFNVC | 100 |
| GLILSAEKSSARKVDENKQLLKIQESVESFRDIYKRFSEYQKEQNSLLMS | 150 |
| NLSTLHIITDSLVPRGSPSRSVKLAGNSSLCPVSGWAIYSKDNSVRIGSK | 200 |
| GDVFVIREPFISCSPLECRTFFLTQGALLNDKHSNGTIKDRSPYRTLMSC | 250 |
| PIGEVPSPYNSRFESVAWSASACHDGINWLTIGISGPDSGAVAVLKYNGI | 300 |
| ITDTIKSWRNNILRTQESECACVNGSCFTIMTDGPSDGQASYKIFRIEKG | 350 |
| KIIKSVEMKAPNYHYEECSCYPDSSEITCVCRDNWHGSNRPWVSFNQNLE | 400 |
| YQMGYICSGVFGDNPRPNDKTGSCGPVSSNGANGVKGFSFKYGNGVWIGR | 450 |
| TKSISSRKGFEMIWDPNGWTGTDNKFSIKQDIVGINEWSGYSGSFVQHPE | 500 |
| LTGLDCIRPCFWVELIRGRPEENTIWTSGSSISFCGVNSDTVGWSWPDGA | 550 |
| ELPFTIDK* | |
| >PiLZ-N1 | |
| (SEQ ID NO: 26) | |
| MLLVNQSHQGFNKEHTSKMVSAIVLYVLLAAAAHSAFAADPHHHHHHLLV | 50 |
| QRMDAKLDLILALIGRLVRQSSLVPRGSPSRSVKLAGNSSLCPVSGWAIY | 100 |
| SKDNSVRIGSKGDVFVIREPFISCSPLECRTFFLTQGALLNDKHSNGTIK | 150 |
| DRSPYRTLMSCPIGEVPSPYNSRFESVAWSASACHDGINWLTIGISGPDS | 200 |
| GAVAVLKYNGIITDTIKSWRNNILRTQESECACVNGSCFTIMTDGPSDGQ | 250 |
| ASYKIFRIEKGKIIKSVEMKAPNYHYEECSCYPDSSEITCVCRDNWHGSN | 300 |
| RPWVSFNQNLEYQMGYICSGVFGDNPRPNDKTGSCGPVSSNGANGVKGFS | 350 |
| FKYGNGVWIGRTKSISSRKGFEMIWDPNGWTGTDNKFSIKQDIVGINEWS | 400 |
| GYSGSFVQHPELTGLDCIRPCFWVELIRGRPEENTIWTSGSSISFCGVNS | 450 |
| DTVGWSWPDGAELPFTIDK* | |
| >MPP-N1 | |
| (SEQ ID NO: 27) | |
| MLLVNQSHQGFNKEHTSKMVSAIVLYVLLAAAAHSAFAADPHHHHHHGDH | 50 |
| YDDELFSDVQDIKTALAKIHEDNQKIISKLESLLLLKGEVESIKKQINRQ | 100 |
| NISISTLEGHLSSIMIAIPGLSLVPRGSPSRSVKLAGNSSLCPVSGWAIY | 150 |
| SKDNSVRIGSKGDVFVIREPFISCSPLECRTFFLTQGALLNDKHSNGTIK | 200 |
| DRSPYRTLMSCPIGEVPSPYNSRFESVAWSASACHDGINWLTIGISGPDS | 250 |
| GAVAVLKYNGIITDTIKSWRNNILRTQESECACVNGSCFTIMTDGPSDGQ | 300 |
| ASYKIFRIEKGKIIKSVEMKAPNYHYEECSCYPDSSEITCVCRDNWHGSN | 350 |
| RPWVSFNQNLEYQMGYICSGVFGDNPRPNDKTGSCGPVSSNGANGVKGFS | 400 |
| FKYGNGVWIGRTKSISSRKGFEMIWDPNGWTGTDNKFSIKQDIVGINEWS | 450 |
| GYSGSFVQHPELTGLDCIRPCFWVELIRGRPEENTIWTSGSSISFCGVNS | 500 |
| DTVGWSWPDGAELPFTIDK* | |
| >ACE-N1 | |
| (SEQ ID NO: 28) | |
| MLLVNQSHQGFNKEHTSKMVSAIVLYVLLAAAAHSAFAADPHHHHHHAVA | 50 |
| DVGDPFLLWKQQMDKWQNEYITDWQYHFEQYKKYQTYRHLDSDSCSGSSL | 100 |
| VPRGSPSRSVKLAGNSSLCPVSGWAIYSKDNSVRIGSKGDVFVIREPFIS | 150 |
| CSPLECRTFFLTQGALLNDKHSNGTIKDRSPYRTLMSCPIGEVPSPYNSR | 200 |
| FESVAWSASACHDGINWLTIGISGPDSGAVAVLKYNGIITDTIKSWRNNI | 250 |
| LRTQESECACVNGSCFTIMTDGPSDGQASYKIFRIEKGKIIKSVEMKAPN | 300 |
| YHYEECSCYPDSSEITCVCRDNWHGSNRPWVSFNQNLEYQMGYICSGVFG | 350 |
| DNPRPNDKTGSCGPVSSNGANGVKGFSFKYGNGVWIGRTKSISSRKGFEM | 400 |
| IWDPNGWTGTDNKFSIKQDIVGINEWSGYSGSFVQHPELTGLDCIRPCFW | 450 |
| VELIRGRPEENTIWTSGSSISFCGVNSDTVGWSWPDGAELPFTIDK* | |

The N1 cysteine mutants, which contain cysteines at different positions in the N1 stalk domain, were cloned into the expression vector pFastBacDual without tetramerization domain. However, the vector still contains the N-terminal signal peptide, the hexa-histidine purification tag as well as the thrombin cleavage site. The construct N1.2 contains part of the stalk domain including the crucial cysteine at position 49 (C49, N1 numbering) which is known to be essential for the formation of stable dimers/tetramers. Construct N1.3 has a mutation at C49, switching the cysteine to alanine resulting in a monomeric protein. The constructs N1.4 (T48C), N1.5 (N50C), N1.6 (T48C+N50C), N1.7 (A76C), N1.8 (Q78C), N1.9 (V81C) and N1.11 (W61C), contain cysteine bonds at different positions in the N1 stalk domain additionally to C49. The construct N1.10 serves as a tetramer control, since it contains a VASP tetramerization domain (1).

The following sequences were used:
Underlined: signal peptide, hexa-histidine tag and thrombin cleavage site
Italics: NA stalk
Bold: introduced mutations

| N1.2 | |
|---|---|
| (SEQ ID NO: 15) | |
| MLLVNQSHQGFNKEHTSKMVSAIVLYVLLAAAAHSAFAADPHHHHHHSLV | 50 |
| PRGSPSRIETCNQSVITYENNTWVNQTYVNISNTNFAAGQSVVSVKLAGN | 100 |
| SSLCPVSGWAIYSKDNSVRIGSKGDVFVIREPFISCSPLECRTFFLTQGA | 150 |
| LLNDKHSNGTIKDRSPYRTLMSCPIGEVPSPYNSRFESVAWSASACHDGI | 200 |
| NWLTIGISGPDSGAVAVLKYNGIITDTIKSWRNNILRTQESECACVNGSC | 250 |
| FTIMTDGPSDGQASYKIFRIEKGKIIKSVEMKAPNYHYEECSCYPDSSEI | 300 |
| TCVCRDNWHGSNRPWVSFNQNLEYQMGYICSGVFGDNPRPNDKTGSCGPV | 350 |
| SSNGANGVKGFSFKYGNGVWIGRTKSISSRKGFEMIWDPNGWTGTDNKFS | 400 |
| IKQDIVGINEWSGYSGSFVQHPELTGLDCIRPCFWVELIRGRPEENTIWT | 450 |
| SGSSISFCGVNSDTVGWSWPDGAELPFTIDK* | |
| N1.3 (C49 A-monomer) | |
| (SEQ ID NO: 16) | |
| MLLVNQSHQGFNKEHTSKMVSAIVLYVLLAAAAHSAFAADPHHHHHHSLV | 50 |
| PRGSPSRIETANQSVITYENNTWVNQTYVNISNTNFAAGQSVVSVKLAGN | 100 |
| SSLCPVSGWAIYSKDNSVRIGSKGDVFVIREPFISCSPLECRTFFLTQGA | 150 |
| LLNDKHSNGTIKDRSPYRTLMSCPIGEVPSPYNSRFESVAWSASACHDGI | 200 |
| NWLTIGISGPDSGAVAVLKYNGIITDTIKSWRNNILRTQESECACVNGSC | 250 |
| FTIMTDGPSDGQASYKIFRIEKGKIIKSVEMKAPNYHYEECSCYPDSSEI | 300 |
| TCVCRDNWHGSNRPWVSFNQNLEYQMGYICSGVFGDNPRPNDKTGSCGPV | 350 |
| SSNGANGVKGFSFKYGNGVWIGRTKSISSRKGFEMIWDPNGWTGTDNKFS | 400 |
| IKQDIVGINEWSGYSGSFVQHPELTGLDCIRPCFWVELIRGRPEENTIWT | 450 |
| SGSSISFCGVNSDTVGWSWPDGAELPFTIDK* | |
| N1.4 (T48C) | |
| (SEQ ID NO: 17) | |
| MLLVNQSHQGFNKEHTSKMVSAIVLYVLLAAAAHSAFAADPHHHHHHSLV | 50 |
| PRGSPSRIECCNQSVITYENNTWVNQTYVNISNTNFAAGQSVVSVKLAGN | 100 |
| SSLCPVSGWAIYSKDNSVRIGSKGDVFVIREPFISCSPLECRTFFLTQGA | 150 |
| LLNDKHSNGTIKDRSPYRTLMSCPIGEVPSPYNSRFESVAWSASACHDGI | 200 |
| NWLTIGISGPDSGAVAVLKYNGIITDTIKSWRNNILRTQESECACVNGSC | 250 |
| FTIMTDGPSDGQASYKIFRIEKGKIIKSVEMKAPNYHYEECSCYPDSSEI | 300 |
| TCVCRDNWHGSNRPWVSFNQNLEYQMGYICSGVFGDNPRPNDKTGSCGPV | 350 |
| SSNGANGVKGFSFKYGNGVWIGRTKSISSRKGFEMIWDPNGWTGTDNKFS | 400 |
| IKQDIVGINEWSGYSGSFVQHPELTGLDCIRPCFWVELIRGRPEENTIWT | 450 |
| SGSSISFCGVNSDTVGWSWPDGAELPFTIDK* | |
| N1.5 (N50C) | |
| (SEQ ID NO: 18) | |
| MLLVNQSHQGFNKEHTSKMVSAIVLYVLLAAAAHSAFAADPHHHHHHSLV | 50 |
| PRGSPSRIETCCQSVITYENNTWVNQTYVNISNTNFAAGQSVVSVKLAGN | 100 |
| SSLCPVSGWAIYSKDNSVRIGSKGDVFVIREPFISCSPLECRTFFLTQGA | 150 |
| LLNDKHSNGTIKDRSPYRTLMSCPIGEVPSPYNSRFESVAWSASACHDGI | 200 |
| NWLTIGISGPDSGAVAVLKYNGIITDTIKSWRNNILRTQESECACVNGSC | 250 |
| FTIMTDGPSDGQASYKIFRIEKGKIIKSVEMKAPNYHYEECSCYPDSSEI | 300 |
| TCVCRDNWHGSNRPWVSFNQNLEYQMGYICSGVFGDNPRPNDKTGSCGPV | 350 |
| SSNGANGVKGFSFKYGNGVWIGRTKSISSRKGFEMIWDPNGWTGTDNKFS | 400 |
| IKQDIVGINEWSGYSGSFVQHPELTGLDCIRPCFWVELIRGRPEENTIWT | 450 |
| SGSSISFCGVNSDTVGWSWPDGAELPFTIDK* | |
| N1.6 (T48C + N50C) | |
| (SEQ ID NO: 19) | |
| MLLVNQSHQGFNKEHTSKMVSAIVLYVLLAAAAHSAFAADPHHHHHHSLV | 50 |
| PRGSPSRIECCCQSVITYENNTWVNQTYVNISNTNFAAGQSVVSVKLAGN | 100 |
| SSLCPVSGWAIYSKDNSVRIGSKGDVFVIREPFISCSPLECRTFFLTQGA | 150 |
| LLNDKHSNGTIKDRSPYRTLMSCPIGEVPSPYNSRFESVAWSASACHDGI | 200 |
| NWLTIGISGPDSGAVAVLKYNGIITDTIKSWRNNILRTQESECACVNGSC | 250 |
| FTIMTDGPSDGQASYKIFRIEKGKIIKSVEMKAPNYHYEECSCYPDSSEI | 300 |
| TCVCRDNWHGSNRPWVSFNQNLEYQMGYICSGVFGDNPRPNDKTGSCGPV | 350 |
| SSNGANGVKGFSFKYGNGVWIGRTKSISSRKGFEMIWDPNGWTGTDNKFS | 400 |
| IKQDIVGINEWSGYSGSFVQHPELTGLDCIRPCFWVELIRGRPEENTIWT | 450 |
| SGSSISFCGVNSDTVGWSWPDGAELPFTIDK* | |
| N1.7 (A76C) | |
| (SEQ ID NO: 20) | |
| MLLVNQSHQGFNKEHTSKMVSAIVLYVLLAAAAHSAFAADPHHHHHHSLV | 50 |
| PRGSPSRIETCNQSVITYENNTWVNQTYVNISNTNFACGQSVVSVKLAGN | 100 |
| SSLCPVSGWAIYSKDNSVRIGSKGDVFVIREPFISCSPLECRTFFLTQGA | 150 |
| LLNDKHSNGTIKDRSPYRTLMSCPIGEVPSPYNSRFESVAWSASACHDGI | 200 |
| NWLTIGISGPDSGAVAVLKYNGIITDTIKSWRNNILRTQESECACVNGSC | 250 |
| FTIMTDGPSDGQASYKIFRIEKGKIIKSVEMKAPNYHYEECSCYPDSSEI | 300 |
| TCVCRDNWHGSNRPWVSFNQNLEYQMGYICSGVFGDNPRPNDKTGSCGPV | 350 |
| SSNGANGVKGFSFKYGNGVWIGRTKSISSRKGFEMIWDPNGWTGTDNKFS | 400 |
| IKQDIVGINEWSGYSGSFVQHPELTGLDCIRPCFWVELIRGRPEENTIWT | 450 |
| SGSSISFCGVNSDTVGWSWPDGAELPFTIDK* | |
| N1.8 (Q78C) | |
| (SEQ ID NO: 21) | |
| MLLVNQSHQGFNKEHTSKMVSAIVLYVLLAAAAHSAFAADPHHHHHHSLV | 50 |
| PRGSPSRIETCNQSVITYENNTWVNQTYVNISNTNFAAGCSVVSVKLAGN | 100 |
| SSLCPVSGWAIYSKDNSVRIGSKGDVFVIREPFISCSPLECRTFFLTQGA | 150 |
| LLNDKHSNGTIKDRSPYRTLMSCPIGEVPSPYNSRFESVAWSASACHDGI | 200 |
| NWLTIGISGPDSGAVAVLKYNGIITDTIKSWRNNILRTQESECACVNGSC | 250 |
| FTIMTDGPSDGQASYKIFRIEKGKIIKSVEMKAPNYHYEECSCYPDSSEI | 300 |
| TCVCRDNWHGSNRPWVSFNQNLEYQMGYICSGVFGDNPRPNDKTGSCGPV | 350 |
| SSNGANGVKGFSFKYGNGVWIGRTKSISSRKGFEMIWDPNGWTGTDNKFS | 400 |
| IKQDIVGINEWSGYSGSFVQHPELTGLDCIRPCFWVELIRGRPEENTIWT | 450 |
| SGSSISFCGVNSDTVGWSWPDGAELPFTIDK* | |
| N1.9 (V81C) | |
| (SEQ ID NO: 22) | |
| MLLVNQSHQGFNKEHTSKMVSAIVLYVLLAAAAHSAFAADPHHHHHHSLV | 50 |
| PRGSPSRIETCNQSVITYENNTWVNQTYVNISNTNFAAGQSVCSVKLAGN | 100 |
| SSLCPVSGWAIYSKDNSVRIGSKGDVFVIREPFISCSPLECRTFFLTQGA | 150 |
| LLNDKHSNGTIKDRSPYRTLMSCPIGEVPSPYNSRFESVAWSASACHDGI | 200 |
| NWLTIGISGPDSGAVAVLKYNGIITDTIKSWRNNILRTQESECACVNGSC | 250 |
| FTIMTDGPSDGQASYKIFRIEKGKIIKSVEMKAPNYHYEECSCYPDSSEI | 300 |
| TCVCRDNWHGSNRPWVSFNQNLEYQMGYICSGVFGDNPRPNDKTGSCGPV | 350 |
| SSNGANGVKGFSFKYGNGVWIGRTKSISSRKGFEMIWDPNGWTGTDNKFS | 400 |
| IKQDIVGINEWSGYSGSFVQHPELTGLDCIRPCFWVELIRGRPEENTIWT | 450 |
| SGSSISFCGVNSDTVGWSWPDGAELPFTIDK* | |
| N1.11 (W61C) | |
| (SEQ ID NO: 23) | |
| MLLVNQSHQGFNKEHTSKMVSAIVLYVLLAAAAHSAFAADPHHHHHHSLV | 50 |
| PRGSPSRIETCNQSVITYENNTCVNQTYVNISNTNFAAGQSVVSVKAAGN | 100 |
| SSLCPVSGWAIYSKDNSVRIGSKGDVFVIREPFISCSPLECRTFFLTQGA | 150 |
| LLNDKHSNGTIKDRSPYRTLMSCPIGEVPSPYNSRFESVAWSASACHDGI | 200 |
| NWLTIGISGPDSGAVAVLKYNGIITDTIKSWRNNILRTQESECACVNGSC | 250 |
| FTIMTDGPSDGQASYKIFRIEKGKIIKSVEMKAPNYHYEECSCYPDSSEI | 300 |
| TCVCRDNWHGSNRPWVSFNQNLEYQMGYICSGVFGDNPRPNDKTGSCGPV | 350 |
| SSNGANGVKGFSFKYGNGVWIGRTKSISSRKGFEMIWDPNGWTGTDNKFS | 400 |
| IKQDIVGINEWSGYSGSFVQHPELTGLDCIRPCFWVELIRGRPEENTIWT | 450 |
| SGSSISFCGVNSDTVGWSWPDGAELPFTIDK* | |

After being cloned into the pFastBacDual expression vector, the constructs were transformed into competent E. coli DH10 bacs and the isolated DNA used to transfect Sf9 (Spodoptera frugiperda) insect cells, following an established protocol (13). The obtained baculoviruses, were then passaged in Sf9 cells to reach higher titers using 3% TNM-FH media (Gemini Bioproducts; 3% Fetal bovine serum (Gibco)). After passaging the virus for three times, the resulting stock solutions were used to infect HighFive cells to express the recombinant neuraminidases. The cells were infected for 3 days at 27° C. without CO₂ using ExpressFive media (Gibco), supplemented with 10% L-glutamine (Gibco) and 1% penicillin/streptomycin (Gibco). Afterwards, the cell supernatant containing the respective soluble neuraminidases was separated from the cells by centrifugation and the protein purified by using an established protocol (13, 14). The proteins were stored at −80° C. until further usage.

### Sodium Dodecyl Sulfate—Polyacrylamide Gel Electrophoresis (SDS-PAGE)

In order to perform a reducing SDS-PAGE, lug of recombinant protein was mixed with 4× Laemmli sample buffer (Biorad) containing 10% β-mercaptoethanol (BME). The samples were heated for 10 minutes at 98° C. and were afterwards loaded on a SDS gradient gel (4-20% Mini-PROTEAN© TGX™ Precast Protein Gels, Biorad). The gel was run for 30 minutes at 200V and was afterwards stained with Coomassie blue fast stain solution for 1 h, shaking at RT. The staining solution was removed and the gel destained by using distilled water.

For performing a non-reducing SDS-Page, the samples were mixed with 4× Laemmli sample buffer and then treated the same way as mentioned above. To cross-link the recombinant proteins, BS3 cross linker (Sigma-Aldrich) was used (13). For thus, the proteins were mixed with the crosslinking reagent as described in the manufacturers manual. Afterwards, the samples were treated the same way as mentioned above.

### NA-Star Assay

To determine potential neuraminidase activity of the different recombinant N1 constructs, an NA-star assay (Thermo Fisher) was performed. The recombinant proteins were diluted to a starting concentration of 10 μg in the provided assay buffer followed by a 1:2 serial dilution. The plates were incubated for 15 minutes at room temperature. The substrate was added and the plates kept in the dark for 30 minutes at 37° C. Afterwards, the provided enhancer solution was added to the wells and the plate immediately read based on luminescence activity using a Synergy Hybrid Reader (BioTek).

### Enzyme-Linked Immunosorbent Assay (ELISA)

To determine the binding activity of the recombinant N1 constructs to a panel of human neuraminidase antibodies, an ELISA was performed. To ensure correct folding of the proteins as well as full accessibility of the respective epitopes, Ni-NTA plates (Qiagen) were used to perform the assay. Therefore, the plates were coated overnight at 4° C. with 50 μl/well of 2 μg/ml recombinant protein diluted in Phosphate-buffered saline (PBS). The following day, the coating solution was removed and the plates blocked with 100 μl/well of 3% milk/PBS-0.01% Tween (PBST) for 1 h at RT. The blocking solution was removed and the primary antibody dilutions prepared at a starting concentration of 30 ug/ml in 1% milk/PBST. A 1:3 serial dilution was performed and the antibodies incubated on the plate for 2 h at RT. Afterwards, the plates were washed 3× with PBST and then incubated with an anti-human IgG secondary HRP labelled antibody (1:3000 in 1% milk/PBST) for 1 h at RT. The plates were washed and 100 μl/well of o-phenylenediamine (OPD) developing solution was added for 10 min at RT. The reaction was stopped by adding 50/well of 3M HCL solution and the plates read at an OD of 490 nm using a Synergy Hybrid Reader (BioTek).

### Mouse Vaccination and Challenge Studies

The protective efficacy of recombinant neuraminidase vaccine candidates was tested in 6-8 week old female BALB/c mice (n=5 per group). The mice were vaccinated intramuscular with 3 ug of the respective recombinant protein (in 50 μl PBS) adjuvanted 1:1 with AddaVax (InvivoGen). An irrelevant hemagglutinin protein (from B/Malaysia/2506/04) was administered to one of the groups as negative control and an N1 protein, containing a VASP tetramerization domain, which has been shown to be protective in in vivo challenge studies was included as a positive control. The mice were vaccinated twice in an interval of 3 weeks. Four weeks after the second vaccination, the mice were challenged with 25×mLD₅₀ (murine lethal dose 50%) of A/Singapore/GP1908/2015 (IVR-180). Survival as well as the weight loss were monitored over 14 days. Mice that lost more than 25% of their initial body weight were humanely euthanized.

### 6.1.3 Results

### Novel Tetramerization Domains

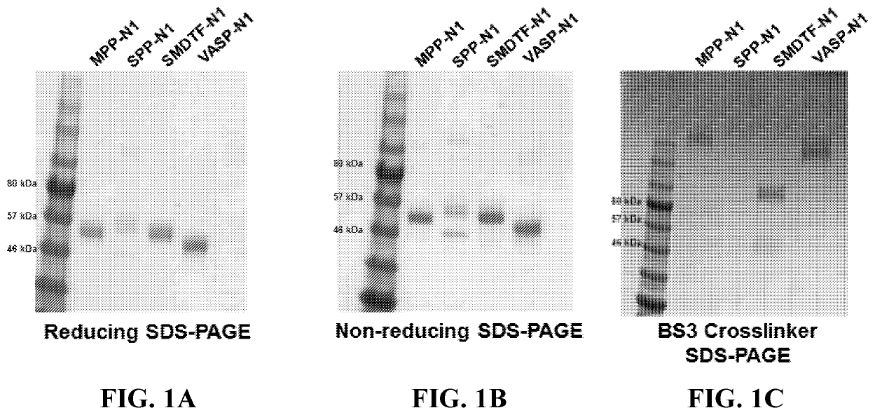

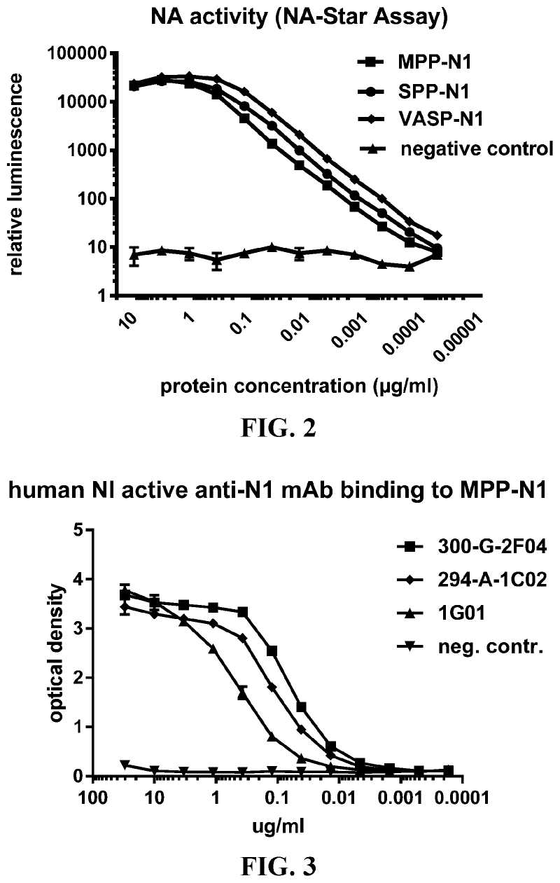

First, three of the constructs plus the positive control (VASP-N1) were expressed and purified. MPP-N1 and SMDTF-N1 (e.g., N1 with a leucine zipper from SEPPALLATA-like MADS domain transcription factor from Arabidopsis thaliana (9)) showed no degradation when analysed on a reducing and non-reducing SDS-PAGE, while SPP-N1 showed slight degradation (FIGS. 1A and 1B). Second, the constructs were tested for formation of tetramers. This was observed for MPP-N1 and SPP-N1 (and the positive control), with MPP-N1 again showing no degradation or secondary bands when cross-linked and run on an SDS-PAGE (FIG. 1C). MPP-N1 behaved identical to VASP-N1 in this assay. Furthermore, successful constructs must have NA activity similar to an N1 tetramer with a known and functional tetramerization domain (VASP-N1 as control). Both the SPP-N1 and MPP-N1 constructs showed similar NA activity. Although the NA activity of MPP-N1 was slightly lower than that of SPP-N1 and VASP-N1, it was still robust (FIG. 2).

Finally, MPP-N1 was tested for recognization by human neuraminidase inhibition active N1 monoclonal antibodies, which was the case (FIG. 3). Both SPP-N1 and MPP-N1 were then moved forward to efficacy testing in animals.

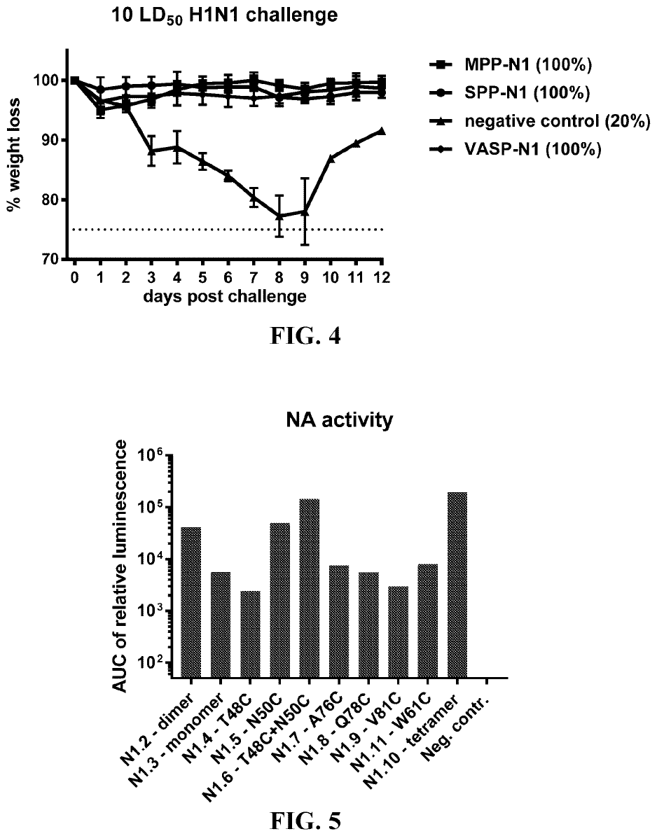

In a simple prime-boost regimen, 3 g of NA was given to mice intramuscularly twice in a three-week interval (with AddaVax as adjuvant). Both constructs provided complete protection against weight loss and mortality comparable to the VASP-N1 constructs when the animals were challenged with 10 50% lethal doses of homologous virus (FIG. 4). The rationale for the 3 μg dose per mouse is based on the typical dose of 1 μg of HA when inactivated vaccines are used in mice. In humans, the dose of recombinant HA in RIV4 is three times higher than in QIV, therefore a three times higher dose of recombinant NA was chosen.

### Cysteine Mutants

The different cysteine mutants described in the methods section were expressed and tested. Neuraminidase activity in an NA-Star assay was used as proxy for tetramer formation. A monomer (N1.3), a dimer (N1.2) and a VASP-stabilized tetramer (N1.10) were used as controls. One construct, N1.6 (T48C+N50C) showed activity comparable to the tetramer (FIG. 5).

### 6.1.4 Discussion

Natural tetramerization domains including the measles virus phosphoprotein tetramerization domain (MPP) (7), the Sendai virus phosphoprotein tetramerization domain (SPP) (8), a tetramerization domain from SEPPALLATA-like MADS domain transcription factor from Arabidopsis thaliana (SMDTF) (9), a PiLZ structure from Xhantomonas campestris (10) and a Dictyocaulus viviparus ACE tetramerization domain (11), all fused to the head domain of the A/Michigan/45/15 N1 (H1N1) were screened. These constructs were characterized based on expression, tetramerization, stability and immunogenicity.

In conclusion, two novel constructs with tetramerization domains from the measles and the Sendai virus phosphoprotein that support formation of functional NA tetramers have been identified. These constructs also provide protection as vaccines in an influenza virus challenge mouse model. Vaccination with the MPP tetramerization domain construct in the mouse model conferred complete protection against a stringent 10×mLD₅₀ challenge.

Furthermore, a neuraminidase stalk cysteine double mutant with NA activity comparable to a corresponding recombinant NA stabilized with a VASP tetramerization domain has been identified. The strong activity of this mutant indicates that it forms a tetramer and could also provide protection as a vaccine in vivo.

Both types of constructs are expressed with a hexa-histidine tag to aid purification but versions of the constructs may be expressed without this tag. A tag-less protein is harder to purify but is more suitable for human clinical trials.

### 6.1.5 References Cited in Example 1 and Background

1. Wohlbold T J, Nachbagauer R, Xu H, Tan G S, Hirsh A, Brokstad K A, Cox R J, Palese P, Krammer F. 2015. Vaccination with Adjuvanted Recombinant Neuraminidase Induces Broad Heterologous, but Not Heterosubtypic, Cross-Protection against Influenza Virus Infection in Mice. MBio 6.
2. Krammer F, Fouchier RAM, Eichelberger M C, Webby R J, Shaw-Saliba K, Wan H, Wilson P C, Compans R W, Skountzou I, Monto A S. 2018. NAction! How Can Neuraminidase-Based Immunity Contribute to Better Influenza Virus Vaccines? MBio 9.
3. Johansson B E, Moran T M, Kilbourne E D. 1987. Antigen-presenting B cells and helper T cells cooperatively mediate intravirionic antigenic competition between influenza A virus surface glycoproteins. Proc Natl Acad Sci USA 84:6869-73.
4. Johansson B E, Kilbourne E D. 1993. Dissociation of influenza virus hemagglutinin and neuraminidase eliminates their intravirionic antigenic competition. J Virol 67:5721-3.
5. Krammer F, Palese P. 2015. Advances in the development of influenza virus vaccines. Nat Rev Drug Discov 14:167-82.
6. Dalakouras T, Smith B, Platis D, Cox M, Labrou N. 2006. Development of recombinant protein-based influenza vaccine. Expression and affinity purification of H1N1 influenza virus neuraminidase. J Chromatogr A 1136:48-56.
7. Communie G, Crépin T, Maurin D, Jensen M R, Blackledge M, Ruigrok R W. 2013. Structure of the tetramerization domain of measles virus phosphoprotein. J Virol 87:7166-9.
8. Tarbouriech N, Curran J, Ruigrok R W, Burmeister W P. 2000. Tetrameric coiled coil domain of Sendai virus phosphoprotein. Nat Struct Biol 7:777-81.
9. Rümpler F, Theiβen G, Melzer R. 2018. A conserved leucine zipper-like motif accounts for strong tetramerization capabilities of SEPALLATA-like MADS-domain transcription factors. J Exp Bot 69:1943-1954.
10. Li T N, Chin K H, Fung K M, Yang M T, Wang A H, Chou S H. 2011. A novel tetrameric PilZ domain structure from xanthomonads. PLoS One 6:e22036.
11. Matthews J B, Lazari O, Davidson A J, Warren S, Selkirk M E. 2006. A tryptophan amphiphilic tetramerization domain-containing acetylcholinesterase from the bovine lungworm, Dictyocaulus viviparus. Parasitology 133:381-7.
12. da Silva D V, Nordholm J, Madjo U, Pfeiffer A, Daniels R. 2013. Assembly of subtype 1 influenza neuraminidase is driven by both the transmembrane and head domains. J Biol Chem 288:644-53.
13. Krammer F, Margine I, Tan G S, Pica N, Krause J C, Palese P. 2012. A carboxy-terminal trimerization domain stabilizes conformational epitopes on the stalk domain of soluble recombinant hemagglutinin substrates. PLoS One 7:e43603.
14. Margine I, Palese P, Krammer F. 2013. Expression of Functional Recombinant Hemagglutinin and Neuraminidase Proteins from the Novel H7N9 Influenza Virus Using the Baculovirus Expression System. J Vis Exp.

### 6.2 Example 2: Stabilization of Neuraminidase (NA) Tetramers of Influenza a Viruses Through Introducing Additional Cysteines to the Stalk Domain of the NA

This example demonstrates the production of recombinant influenza viruses engineered to express a recombinant neuraminidase in which additional disulfide bonds were introduced into the stalk region of the viral NA through cysteine substitution(s) or insertion(s). This example demonstrates that introduction of cysteines appears to stabilize the tetrameric neuraminidiase of the inactivated purified A/Puerto Rico/08/1934 (PR8) H1N1 virus under conditions, when the viral proteins are resolved on a SDS-PAGE. This observation also holds true for a more clinically relevant A/Hong Kong/4801/2014 (HK14) H3N2 strain.

### 6.2.1 Materials and Methods

### Plasmids

To introduce additional cysteine(s) into the stalk region of NA, amino acid substitution(s) or insertion were performed through PCR mutagenesis using pDZ N1 plasmid for the A/Puerto Rico/08/1934 or pDZ N2 plasmid for the A/Hong Kong/4801/2014 strain as templates. The modified NA gene segments were cloned into linearized vector, which was generated by SapI restriction enzyme (New England Biolabs, Inc.) digestion of the pDZ ambisense plasmid (1). In-Fusion cloning (Takara Bio, Kusatsu, Shiga Prefecture, Japan) was performed. The recombination products were transformed into Escherichia coli DH5a competent cells (Thermo Fisher Scientific) and plasmids were purified using QIAprep Spin Miniprep kit (Qiagen). The pRS PR8 6 segment plasmid used to rescue recombinant influenza viruses was generated through deleting NA cassette from pRS PR8 7 segment plasmid which has been described previously (2). Briefly, the pRS PR8 7 segment was digested using NotI (New England Biolabs, Inc.) to specifically remove the NA cassette, the linearized plasmid was re-ligated using T4 DNA ligase (New England Biolabs, Inc.) and transformed into MAX Efficiency Stbl2 Competent cells (Invitrogen) according to the manufacturers' protocols. Plasmids were purified from subsequent colonies using the QIAprep spin miniprep kit (Qiagen).

### SEQUENCES

Amino acid sequences of the wild type (WT) and mutant PR8 N1 or HK14 N2 are shown below. These sequences include signal peptide. The amino acid substitutions are in bold and underlined. The amino acid insertions are in bold, italics and underlined. C49 in PR8 N1 and C53 in HK14 N2 are double underlined and italicized.

| >PR8 N1 WT | |
|---|---|
| (SEQ ID NO: 6) | |
| 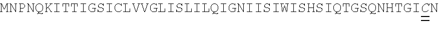 | 50 |
| QNIITYKNSTWVKDTTSVILTGNSSLCPIRGWAIYSKDNSIRIGSKGDVF | 100 |
| VIREPFISCSHLECRTFFLTQGALLNDKHSNGTVKDRSPYRALMSCPVGE | 150 |
| APSPYNSRFESVAWSASACHDGMGWLTIGISGPDNGAVAVLKYNGIITET | 200 |
| IKSWRKKILRTQESECACVNGSCFTIMTDGPSDGLASYKIFKIEKGKVTK | 250 |
| SIELNAPNSHYEECSCYPDTGKVMCVCRDNWHGSNRPWVSFDQNLDYQIG | 300 |
| YICSGVFGDNPRPEDGTGSCGPVYVDGANGVKGFSYRYGNGVWIGRTKSH | 350 |
| SSRHGFEMIWDPNGWTETDSKFSVRQDVVAMTDWSGYSGSFVQHPELTGL | 400 |
| DCMRPCFWVELIRGRPKEKTIWTSASSISFCGVNSDTVDWSWPDGAELPF | 450 |
| SIDK* | |
| >PR8 N1 I48C | |
| (SEQ ID NO: 7) | |
| 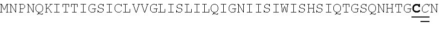 | 50 |
| QNIITYKNSTWVKDTTSVILTGNSSLCPIRGWAIYSKDNSIRIGSKGDVF | 100 |
| VIREPFISCSHLECRTFFLTQGALLNDKHSNGTVKDRSPYRALMSCPVGE | 150 |
| APSPYNSRFESVAWSASACHDGMGWLTIGISGPDNGAVAVLKYNGIITET | 200 |
| IKSWRKKILRTQESECACVNGSCFTIMTDGPSDGLASYKIFKIEKGKVTK | 250 |
| SIELNAPNSHYEECSCYPDTGKVMCVCRDNWHGSNRPWVSFDQNLDYQIG | 300 |
| YICSGVFGDNPRPEDGTGSCGPVYVDGANGVKGFSYRYGNGVWIGRTKSH | 350 |
| SSRHGFEMIWDPNGWTETDSKFSVRQDVVAMTDWSGYSGSFVQHPELTGL | 400 |
| DCMRPCFWVELIRGRPKEKTIWTSASSISFCGVNSDTVDWSWPDGAELPF | 450 |
| SIDK* | |
| >PR8 N1 N50C | |
| (SEQ ID NO: 8) | |
| 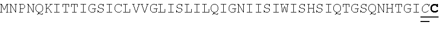 | 50 |
| QNIITYKNSTWVKDTTSVILTGNSSLCPIRGWAIYSKDNSIRIGSKGDVF | 100 |
| VIREPFISCSHLECRTFFLTQGALLNDKHSNGTVKDRSPYRALMSCPVGE | 150 |
| APSPYNSRFESVAWSASACHDGMGWLTIGISGPDNGAVAVLKYNGIITET | 200 |
| IKSWRKKILRTQESECACVNGSCFTIMTDGPSDGLASYKIFKIEKGKVTK | 250 |
| SIELNAPNSHYEECSCYPDTGKVMCVCRDNWHGSNRPWVSFDQNLDYQIG | 300 |
| YICSGVFGDNPRPEDGTGSCGPVYVDGANGVKGFSYRYGNGVWIGRTKSH | 350 |
| SSRHGFEMIWDPNGWTETDSKFSVRQDVVAMTDWSGYSGSFVQHPELTGL | 400 |
| DCMRPCFWVELIRGRPKEKTIWTSASSISFCGVNSDTVDWSWPDGAELPF | 450 |
| SIDK* | |
| >PR8 N1 61insertC | |
| (SEQ ID NO: 9) | |
| 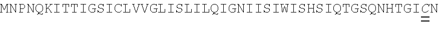 | 50 |
| 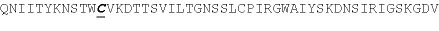 | 100 |
| FVIREPFISCSHLECRTFFLTQGALLNDKHSNGTVKDRSPYRALMSCPVG | 150 |
| EAPSPYNSRFESVAWSASACHDGMGWLTIGISGPDNGAVAVLKYNGIITE | 200 |
| TIKSWRKKILRTQESECACVNGSCFTIMTDGPSDGLASYKIFKIEKGKVT | 250 |
| KSIELNAPNSHYEECSCYPDTGKVMCVCRDNWHGSNRPWVSFDQNLDYQI | 300 |
| GYICSGVFGDNPRPEDGTGSCGPVYVDGANGVKGFSYRYGNGVWIGRTKS | 350 |
| HSSRHGFEMIWDPNGWTETDSKFSVRQDVVAMTDWSGYSGSFVQHPELTG | 400 |
| LDCMRPCFWVELIRGRPKEKTIWTSASSISFCGVNSDTVDWSWPDGAELP | 450 |
| FSIDK* | |
| >PR8 N1 I48C_N50C | |
| (SEQ ID NO: 10) | |
| 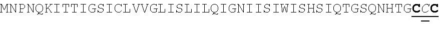 | 50 |
| QNIITYKNSTWVKDTTSVILTGNSSLCPIRGWAIYSKDNSIRIGSKGDVF | 100 |
| VIREPFISCSHLECRTFFLTQGALLNDKHSNGTVKDRSPYRALMSCPVGE | 150 |
| APSPYNSRFESVAWSASACHDGMGWLTIGISGPDNGAVAVLKYNGIITET | 200 |
| IKSWRKKILRTQESECACVNGSCFTIMTDGPSDGLASYKIFKIEKGKVTK | 250 |
| SIELNAPNSHYEECSCYPDTGKVMCVCRDNWHGSNRPWVSFDQNLDYQIG | 300 |
| YICSGVFGDNPRPEDGTGSCGPVYVDGANGVKGFSYRYGNGVWIGRTKSH | 350 |
| SSRHGFEMIWDPNGWTETDSKFSVRQDVVAMTDWSGYSGSFVQHPELTGL | 400 |
| DCMRPCFWVELIRGRPKEKTIWTSASSISFCGVNSDTVDWSWPDGAELPF | 450 |
| SIDK* | |
| >PR8 N1 I48C_61insertC | |
| (SEQ ID NO: 11) | |
| 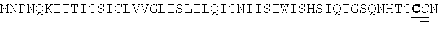 | 50 |
| 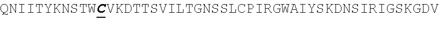 | 100 |
| FVIREPFISCSHLECRTFFLTQGALLNDKHSNGTVKDRSPYRALMSCPVG | 150 |
| EAPSPYNSRFESVAWSASACHDGMGWLTIGISGPDNGAVAVLKYNGIITE | 200 |
| TIKSWRKKILRTQESECACVNGSCFTIMTDGPSDGLASYKIFKIEKGKVT | 250 |
| KSIELNAPNSHYEECSCYPDTGKVMCVCRDNWHGSNRPWVSFDQNLDYQI | 300 |
| GYICSGVFGDNPRPEDGTGSCGPVYVDGANGVKGFSYRYGNGVWIGRTKS | 350 |
| HSSRHGFEMIWDPNGWTETDSKFSVRQDVVAMTDWSGYSGSFVQHPELTG | 400 |
| LDCMRPCFWVELIRGRPKEKTIWTSASSISFCGVNSDTVDWSWPDGAELP | 450 |
| FSIDK* | |
| >HK14 N2 WT | |
| (SEQ ID NO: 12) | |
| MNPNQKIITIGSVSLTISTICFFMQIAILITTVTLHFKQYEFNSPPNNQV | 50 |
| 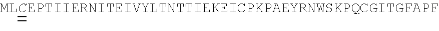 | 100 |
| SKDNSIRLSAGGDIWVTREPYVSCDPDKCYQFALGQGTTLNNVHSNNTVR | 150 |
| DRTPYRTLLMNELGVPFHLGTKQVCIAWSSSSCHDGKAWLHVCITGDDKN | 200 |
| ATASFIYNGRLVDSVVSWSKDILRTQESECICINGTCTVVMTDGSASGKA | 250 |
| DTKILFIEEGKIVHTSTLSGSAQHVEECSCYPRYPGVRCVCRDNWKGSNR | 300 |
| PIVDINIKDHSIVSSYVCSGLVGDTPRKNDSSSSSHCLDPNNEEGGHGVK | 350 |
| GWAFDDGNDVWMGRTINETSRLGYETFKVIEGWSNPKSKLQTNRQVIVDR | 400 |
| GDRSGYSGIFSVEGKSCINRCFYVELIRGRKEETEVLWTSNSIVVFCGTS | 450 |
| GTYGTGSWPDGADLNLMPI* | |
| >HK14 N2 L52C | |
| (SEQ ID NO: 13) | |
| MNPNQKIITIGSVSLTISTICFFMQIAILITTVTLHFKQYEFNSPPNNQV | 50 |
| 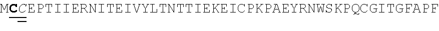 | 100 |
| SKDNSIRLSAGGDIWVTREPYVSCDPDKCYQFALGQGTTLNNVHSNNTVR | 150 |
| DRTPYRTLLMNELGVPFHLGTKQVCIAWSSSSCHDGKAWLHVCITGDDKN | 200 |
| ATASFIYNGRLVDSVVSWSKDILRTQESECICINGTCTVVMTDGSASGKA | 250 |
| DTKILFIEEGKIVHTSTLSGSAQHVEECSCYPRYPGVRCVCRDNWKGSNR | 300 |
| PIVDINIKDHSIVSSYVCSGLVGDTPRKNDSSSSSHCLDPNNEEGGHGVK | 350 |
| GWAFDDGNDVWMGRTINETSRLGYETFKVIEGWSNPKSKLQTNRQVIVDR | 400 |
| GDRSGYSGIFSVEGKSCINRCFYVELIRGRKEETEVLWTSNSIVVFCGTS | 450 |
| GTYGTGSWPDGADLNLMPI* | |
| >HK14 N2 L52C_E54C | |
| (SEQ ID NO: 14) | |
| MNPNQKIITIGSVSLTISTICFFMQIAILITTVTLHFKQYEFNSPPNNQV | 50 |
| 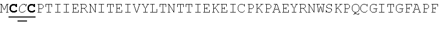 | 100 |
| SKDNSIRLSAGGDIWVTREPYVSCDPDKCYQFALGQGTTLNNVHSNNTVR | 150 |
| DRTPYRTLLMNELGVPFHLGTKQVCIAWSSSSCHDGKAWLHVCITGDDKN | 200 |
| ATASFIYNGRLVDSVVSWSKDILRTQESECICINGTCTVVMTDGSASGKA | 250 |
| DTKILFIEEGKIVHTSTLSGSAQHVEECSCYPRYPGVRCVCRDNWKGSNR | 300 |
| PIVDINIKDHSIVSSYVCSGLVGDTPRKNDSSSSSHCLDPNNEEGGHGVK | 350 |
| GWAFDDGNDVWMGRTINETSRLGYETFKVIEGWSNPKSKLQTNRQVIVDR | 400 |
| GDRSGYSGIFSVEGKSCINRCFYVELIRGRKEETEVLWTSNSIVVFCGTS | 450 |
| GTYGTGSWPDGADLNLMPI* | |

### Cells

Human embryonic kidney 293T (HEK 293T) cells were maintained in Dulbecco's Modified Eagle's medium (DMEM; Gibco) containing 10% (vol/vol) fetal bovine serum (FBS) and 100 unit/mL of penicillin/streptomycin (PS; Gibco) at 37° C. with 5% CO2. Madin-Darby canine kidney (MDCK) cells were grown in Minimum Essential Medium (MEM; Gibco) supplemented with 10% (vol/vol) FBS, 2 mM of L-glutamine (Gibco), 0.15% (w/vol) of sodium bicarbonate (Corning), 20 mM of 2-[4-(2-hydroxyethyl)piperazin-1-yl]ethanesulfonic acid (HEPES, Gibco), and 100 unit/mL of PS at 37° C. with 5% CO₂.

### Rescue of Recombinant Influenza Viruses

Each well of poly-D lysine (Sigma-Aldrich) coated 6-well plates of HEK 293T cells was transfected with 2.1 μg of pRS PR8 6 segment, 0.7 μg of pDZ HA and 0.7 μg modified pDZ NA using TransIT LT1 transfection reagent (Mirus Bio). Transfected cells were incubated at 37° C. Forty-eight hours post-transfection, supernatants together with scraped cells were collected and briefly homogenized through several syringe strokes. Two-hundred microliters of cells and supernatant mixture were injected into the allantoic cavity of 8-day old embryonated chicken eggs (Charles River). Injected eggs were incubated at 33° C. for 3 days and then cooled at 4° C. overnight. Allantoic fluids were subsequently collected and clarified by low speed centrifugation. An HA assay was performed using 0.5% turkey red blood cells to examine the presence of rescued virus from the clarified allantoic fluids. HA positive allantoic fluid samples were used to plaque-purify virus on MDCK cells. Plaques grown on MDCK cells were picked and re-suspended in PBS and further amplified again in 10-day old embryonated chicken eggs. RNA was extracted from allantoic fluids containing the plaque-purified virus using QIAamp Viral RNA Mini Kit (Qiagen). One-step RT-PCR was performed to amplify DNA of the NA segment using the SuperScript™ III One-Step RT-PCR System with Platinum™ Taq DNA Polymerase (Thermo Fisher Scientific) and NA specific primers. DNA was gel-purified and sequenced by Sanger sequencing (Genewiz). All the H3N2 viruses were also rescued in the PR8 backbone (6 genomic segments except HA and NA are from PR8)

### Inactivation and Purification of Influenza Viruses

Influenza viruses were grown in 10-day old embryonated chicken eggs at 37° C. for two days, and were then cooled at 4° C. overnight. Allantoic fluids were collected and clarified by low speed centrifugation. Viruses in the clarified allantoic fluids were inactivated with 0.03% methanol-free formaldehyde for 72 h at 4° C. with rocking. Viruses were then pelleted through a 30% sucrose cushion in NTE buffer (100 mM NaCl, 10 mM Tris-HCl, 1 mM EDTA, pH 7.4) by centrifugation in a Beckman L7-65 ultracentrifuge at 25,000 rpm for 2 h at 4° C. using a Beckman SW28 rotor (Beckman Coulter, Brea, CA, USA). Pellets were collected in PBS (pH 7.4), and protein content was quantified using the bicinchoninic acid (BCA) assay (Thermo Fisher Scientific).

### Western Blots

Inactivated purified virus preparations were lysed by mixing with 2×Novex™ Tris-Glycine SDS Sample buffer (Thermo Fisher Scientific). For reducing condition, 10×NuPAGE™ Sample Reducing Agent (Thermo Fisher Scientific) was included. Two micrograms of reducing or non-reducing virus preparations in a total volume of 10 μl were boiled for 5 min to denature the proteins and resolved onto 10% or 8%-16% polyacrylamide gels (Bio-rad Laboratories) under denaturing conditions in the presence of sodium dodecyl sulfate (SDS). Novex™ Sharp Pre-stained Protein Standard (3.5-260 kDa, Thermo Fisher Scientific) or Color Pre-stained Protein Standard, Broad Range standard (10-250 kDa, New England Biolabs, inc.) was used as a protein size marker. The separated viral proteins were then transferred onto polyvinylidene difluoride (PVDF) membranes. The membranes were blocked for 1 h using PBS containing 5% (wt/vol) dry milk powder and were washed three times with PBS containing 0.01% (vol/vol) Tween 20 (PBST). The primary antibodies were mouse anti-N1 monoclonal antibody 4A5 (3) (1 μg per ml) or anti-N2 polyclonal serum raised in guinea pigs (generated in-house; 1: 2,000 dilution). The primary antibodies were diluted in PBS containing 1% (wt/vol) bovine serum albumin (BSA) and incubated on the membranes overnight at 4′C. The membranes were washed three times with PBST and were incubated for 1 h with secondary horseradish peroxidase (HRP)-labeled antibodies (anti-mouse [GE Healthcare] or anti-guinea pig [Thermofisher Fisher]) diluted 1: 3,000 in PBS containing 1% (wt/vol) BSA according to the manufacturer's recommendations. After three washes with PBST, developing solution (Pierce ECL Western blotting substrate; Thermo Fisher Scientific) was added to the membranes, which were subsequently developed in a ChemiDoc MP imaging system (Bio-Rad Laboratories).

### NA-Fluor Influenza Neuraminidase Assay

To measure potential neuraminidase activity of different mutant viruses, a fluorescence-based NA activity assay was performed. The viruses were diluted to 2 ng/μl in PBS resulting in 5 ng in 25 μl PBS. Twenty-five microliters of assay buffer (33.3 mM 2-(N-morpholino)ethanesulfonic acid (MES) and 4 mM CaCl₂), pH 6.5) were added to each well of a black 96-well plate. Five nanograms of virus in 25 μl of PBS were added to each well in triplicates. The 2′-(4-Methylumbelliferyl)-α-D-N-acetylneuraminic acid (MUNANA) (Sigma-Aldrich) substrate was prepared in assay buffer at a concentration of 200 uM. Fifty microliters of substrate were added to each well and incubated in dark at 37° C. for 1 hour. The reaction was stopped by adding 100 μl of stop solution (0.1 glycine in 20% ethanol, pH 10.7). The plates were read at excitation wavelength 365 nm and emission wavelength 465 nm to measure fluorescence signals using a Synergy Hybrid Reader (BioTek).

### 6.2.2 Results

Introduction of Additional Cysteines into the Stalk Region of N1 Neuraminidase

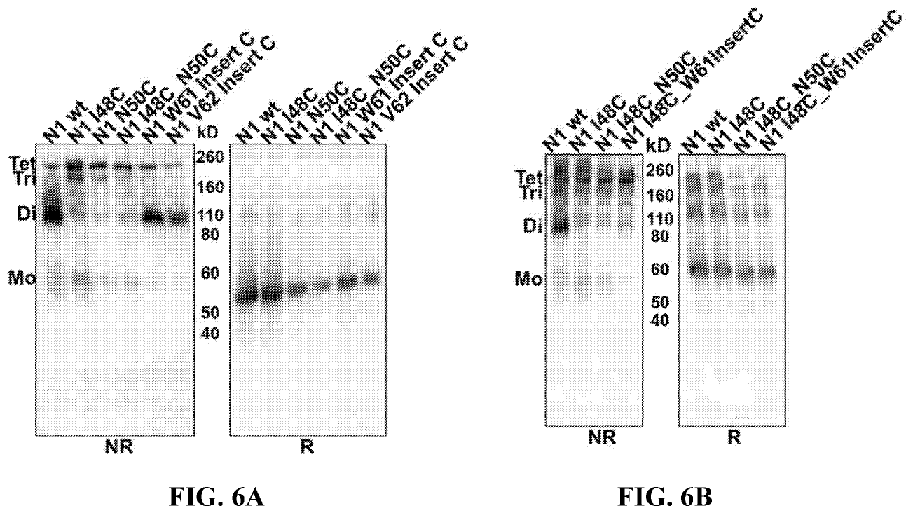

It was previously described that natural cysteine(s) in the stalk region contributed to dimer and tetramer formation of the influenza virus NA proteins as well as their enzymatic activity (4, 5). The manufacturing process of IIVs involve possibly harsh physical and chemical treatments (6), which could be potentially detrimental to the structure of the NA. To further stabilize NA tetramers, additional cysteines (disulfide bonds) were introduced into the stalk region by amino acids substitutions or insertions, as such an approach has been shown to benefit tetramer formation of the NA when it was overexpressed in mammalian cells (5). As a proof of principle study, mutagenesis in the PR8 N1 was first performed, which included 148C, N50C and I48C_N50C amino acid substitutions as well as insertions of cysteines after W61 and V62 based on our transposons mutagenesis study (unpublished data). Recombinant PR8 cysteine mutant viruses were rescued and amplified in embryonated chicken eggs. Viruses were inactivated using 0.03% formaldehyde and purified through 30% sucrose cushion. To visualize NA contents in virus preparations, 2 μg of each virus were resolved on SDS-PAGE under non-reducing (NR) or reducing (R) conditions. A western blot was performed using N1-specific mouse monoclonal antibody 4A5 (3) (FIG. 6A). Mutant I48C, N50C and I48C_N50C viruses showed more trimeric and tetrameric forms of the NA according to the size of the protein under non-reducing condition. Mutant W61insertC and V62insertC viruses showed similar oligomerization patterns as the WT virus, most of which are dimers. To further avoid the trimeric form of NA, 148C was combined with W61insertC to generate I48C_W61insertC mutant. It appears the I48C W61insertC mutant had more stable NA tetramers but fewer NA trimers (FIG. 6B).

Introduction of Additional Cysteines into the Stalk Region of N2 Neuraminidase

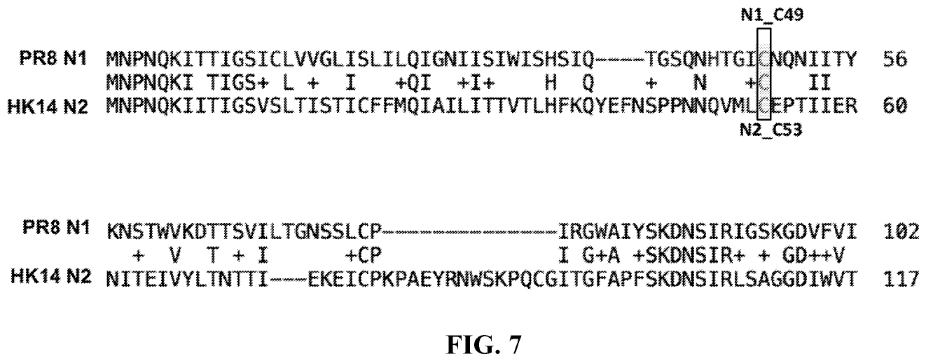

To apply the same approach to another influenza virus subtype and to a more clinically relevant strain than PR8, an amino acid sequence alignment of the PR8 N1 with HK14 N2 proteins was performed (FIG. 7). The HK14 N2 also has a natural cysteine (C53) that corresponds to the I49C of PR8 N1. Similar mutagenesis was done to generate N2 mutants that are equivalent to N1 I48C and I48C_N50C. These mutants were defined as L52C and L52C_E54C by amino acid sequence alignment (FIG. 7)

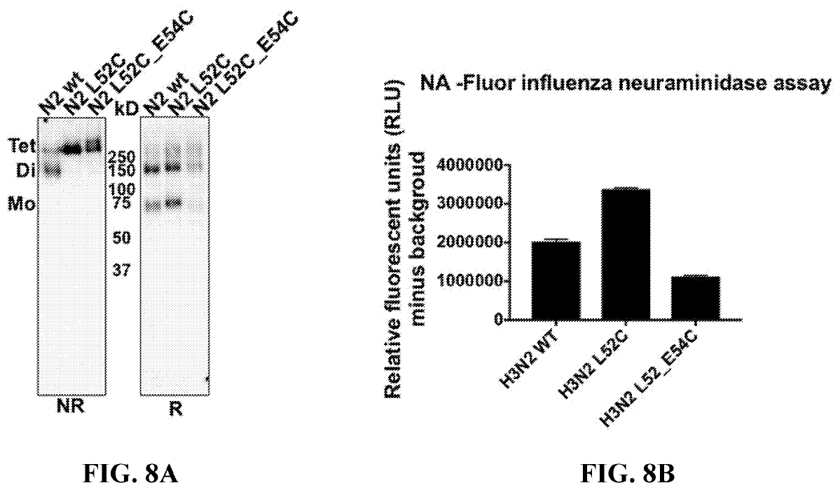

The WT and mutant H3N2 viruses were rescued in the PR8 backbone (6 segments are from PR8 except HA and NA). These viruses were inactivated and purified using the same approach described for PR8. Again, the virus preparations were resolved onto non-reducing (NR) and reducing (R) SDS-PAGE followed by a western blot using guinea pig antisera raised against HK14 N2 recombinant protein to detect NA contents (FIG. 8A). Both L52C and L52C_E54C showed more tetrameric contents than the WT virus.

To measure the potential NA activity of H3N2 mutant viruses, a fluorescence-based NA activity assay was used. As plate reader used has a saturation limit for fluorescence signals, the viruses were diluted to 2 ng/μl in PBS, resulting in 5 ng of virus being tested for NA activity. The L52C mutant instead of L52C_E54C mutant exhibited higher fluorescence signals than WT virus (FIG. 8B). However, this is only in the context of formaldehyde inactivation and purification through 30% sucrose, which might not necessarily be the conditions of NA tetramer disruption. virus preparations will be processed as close to industrial manufacturing procedures as possible to identify H1N1 and H3N2 cysteine mutants that have the highest enzymatic activity in the final IIV products.

### 6.2.3 Discussion

Mutations in the NA stalk region that stabilized the tetrameric form of both N1 and N2 in formaldehyde inactivated purified virus preparations were identified. Those mutants include N1 I48C, N1 N50C, N1 I48C_N50C and N1 I48C_W61insertC; N2 L52C and N2 L52_E54C. The H3N2 L52C NA mutant seems to show higher NA activity in a fluorescence-based NA activity assay.

### 6.2.4 References Cited in Example 2

1. Martinez-Sobrido L, Garcia-Sastre A. 2010. Generation of recombinant influenza virus from plasmid DNA. J Vis Exp doi:10.3791/2057.
2. Fulton B O, Sun W, Heaton N S, Palese P. 2018. The Influenza B Virus Hemagglutinin Head Domain Is Less Tolerant to Transposon Mutagenesis than That of the Influenza A Virus. J Virol 92.
3. Wohlbold T J, Nachbagauer R, Xu H, Tan G S, Hirsh A, Brokstad K A, Cox R J, Palese P, Krammer F. 2015. Vaccination with adjuvanted recombinant neuraminidase induces broad heterologous, but not heterosubtypic, cross-protection against influenza virus infection in mice. MBio 6:e02556.
4. Basler C F, Garcia-Sastre A, Palese P. 1999. Mutation of neuraminidase cysteine residues yields temperature-sensitive influenza viruses. J Virol 73:8095-103.
5. da Silva D V, Nordholm J, Madjo U, Pfeiffer A, Daniels R. 2013. Assembly of subtype 1 influenza neuraminidase is driven by both the transmembrane and head domains. J Biol Chem 288:644-53.
6. Kon T C, Onu A, Berbecila L, Lupulescu E, Ghiorgisor A, Kersten G F, Cui Y Q, Amorij J P, Van der Pol L. 2016. Influenza Vaccine Manufacturing: Effect of Inactivation, Splitting and Site of Manufacturing. Comparison of Influenza Vaccine Production Processes. PLoS One 11:e0150700.

### 6.3 Example 3: Recombinant Neuraminidase-Based Constructs

This example demonstrates the ability of recombinant NA proteins, which comprise the globular head domain of influenza virus NA and the measles virus phosphoprotein tetramerization domain, to induce an immune response in a murine model that is protective against influenza virus challenge.

Recombinant NA proteins. The N1-MPP is described in Example 1. The recombinant N2-MPP protein, which comprises the globular head domain of influenza virus A/Kansas/14/2017 and MPP tetramerization domain, was expressed using a modified pFastBacDual expression vector. Similarly, the recombinant B-MPP protein, which comprises the globular head domain of influenza virus B/Colorado/06/2017 and MPP tetramerization domain, was expressed using the pFastBacDual expression vector. The nucleotide and amino acid sequences of the recombinant N2-MPP and B-MPP proteins are provided below. The amino acid sequence of N1-MPP is provided in SEQ ID NO:27.

| SEQ ID | | |
|---|---|---|
| NO: | Construct | Sequence |
| 59 | N2-MPP | atgctgctcgtcaaccaatcccaccagggcttcaacaaggaacacacttctaagatggt |
| | | ctccgctatcgtgctctacgtgctgctcgctgccgctgcccactcagctttcgctgccga |
| | | cccacaccaccaccaccaccacggcgatcactacgacgacgaactgttctccgacgt |
| | | gcaggacatcaagaccgctctggctaagatccacgaggacaaccagaagatcatctc |
| | | caagctggaatccctgctgctgctgaagggcgaagtcgagtccatcaagaagcagatc |
| | | aaccgccagaacatctccatctccaccttggagggtcacctgtcctccatcatgatcgct |
| | | atccctggcctgtctctcgtgcccaggggatcaccttctagaATATGCCCCAA |
| | | ACCAGCAGAATACAGAAATTGGTCAAAACCGCAATG |
| | | TGGCATTACAGGATTTGCACCTTTCTCTAAGGACAAT |
| | | TCGATTAGGCTTTCCGCTGGTGGGGACATCTGGGTG |
| | | ACAAGAGAACCTTATGTGTCATGCGATCCTGACAAG |
| | | TGTTATCAATTTGCCCTTGGACAGGGAACAACAATA |
| | | AACAACGTGCATTCAAATAACACAGCACGTGATAGG |
| | | ACCCCTCATCGGACTCTATTGATGAATGAGTTGGGT |
| | | GTTCCTTTCCATCTGGGGACCAAGCAAGTGTGCATA |
| | | GCATGGTCCAGCTCAAGTTGTCACGATGGAAAAGCA |
| | | TGGCTGCATGTTTGTATAACGGGGGATGATAAAAAT |
| | | GCAACTGCTAGTTTCATTTACAATGGGAGGCTTGTA |
| | | GATAGTGTTGTTTCATGGTCCAAAGATATTCTCAGGA |
| | | CCCAGGAGTCAGAATGCGTTTGTATCAATGGAACTT |
| | | GTACAGTAGTAATGACTGATGGAAATGCTACAGGAA |
| | | AAGCTGATACTAAAATATTATTCATTGAGGAGGGGA |
| | | AAATCGTTCATACTAGCAAATTGTCAGGAAGTGCTC |
| | | AGCATGTCGAAGAGTGCTCTTGCTATCCTCGATACCC |
| | | TGGTGTCAGATGTGTCTGCAGAGACAACTGGAAAGG |
| | | ATCCAACCGGCCCATCGTAGATATAAACATAAAGGA |
| | | TCATAGCATTGTTTCCAGTTATGTGTGTTCAGGACTT |
| | | GTTGGAGACACACCCAGAAAAACCGACAGCTCCAGC |
| | | AGCAGCCATTGCTTGAATCCTAACAATGAAAAAGGT |
| | | GGTCATGGAGTGAAAGGCTGGGCCTTTGATGATGGA |
| | | AATGACGTGTGGATGGGGAGAACAATCAACGAGAC |
| | | GTCACGCTTAGGGTATGAAACCTTCAAAGTCGTTGA |
| | | AGGCTGGTCCAACCCTAAGTCCAAATTGCAGATAAA |
| | | TAGGCAAGTCATAGTTGACAGAGGTGATAGGTCCGG |
| | | TTATTCTGGTATTTTCTCTGTTGAAGGCAAAAGCTGC |
| | | ATCAATCGGTGCTTTTATGTGGAGTTGATTAGGGGA |
| | | AGAAAAGAGGAAACTGAAGTCTTGTGGACCTCAAAC |
| | | AGTATTGTTGTGTTTTGTGGCACCTCAGGTACATATG |
| | | GAACAGGCTCATGGCCTGATGGGGCGGACCTCAATC |
| | | TCATGCATATATAA |
| 60 | N2-MPP | MLLVNQSHQGFNKEHTSKMVSAIVLYVLLAAAAHSAFAAD |
| | | PHHHHHHGDHYDDELFSDVQDIKTALAKIHEDNQKIIS |
| | | KLESLLLLKGEVESIKKQINRQNISISTLEGHLSSIMIAIP |
| | | GLSLVPRGSPSRICPKPAEYRNWSKPQCGITGFAPFS |
| | | KDNSIRLSAGGDIWVTREPYVSCDPDKCYQFALGQG |
| | | TTINNVHSNNTARDRTPHRTLLMNELGVPFHLGTK |
| | | QVCIAWSSSSCHDGKAWLHVCITGDDKNATASFIYN |
| | | GRLVDSVVSWSKDILRTQESECVCINGTCTVVMTDG |
| | | NATGKADTKILFIEEGKIVHTSKLSGSAQHVEECSCY |
| | | PRYPGVRCVCRDNWKGSNRPIVDINIKDHSIVSSYVC |
| | | SGLVGDTPRKTDSSSSSHCLNPNNEKGGHGVKGWA |
| | | FDDGNDVWMGRTINETSRLGYETFKVVEGWSNPKS |
| | | KLQINRQVIVDRGDRSGYSGIFSVEGKSCINRCFYVE |
| | | LIRGRKEETEVLWTSNSIVVFCGTSGTYGTGSWPDG |
| | | ADLNLMHI* |
| | | (signal peptide and his tag are italicized; MPP tetramerization |
| | | domain is underlined; thrombin cleavage site plus two amino |
| | | acid residues is double underline; NA globular domain of |
| | | influenza virus A/Kansas/14/2017 is bold) |
| 61 | B-MPP | atgctgctcgtcaaccaatcccaccagggcttcaacaaggaacacacttctaagatggt |
| | | ctccgctatcgtgctctacgtgctgctcgctgccgctgcccactcagctttcgctgccga |
| | | cccacaccaccaccaccaccacggcgatcactacgacgacgaactgttctccgacgt |
| | | gcaggacatcaagaccgctctggctaagatccacgaggacaaccagaagatcatctc |
| | | caagctggaatccctgctgctgctgaagggcgaagtcgagtccatcaagaagcagatc |
| | | aaccgccagaacatctccatctccaccttggagggtcacctgtcctccatcatgatcgct |
| | | atccctggcctgtctctcgtgcccaggggatcaccttctagacttcttctcccagaaccg |
| | | gagtggacatacccgcgtttatcttgcccgggctcaacctttcagaaagcactcctaatt |
| | | agccctcatagattcggagaaaccaaaggaaactcagctcccttgataataagggaac |
| | | cttttgttgcttgtggaccaaatgaatgcaaacactttgctttaacccattatgcagcccaa |
| | | ccagggggatactacaatggaacaagaggagacagaaacaagctgaggcatctaatt |
| | | tcagtcaaattgggcaaaatcccaacagtagagaactccattttccacatggcagcatg |
| | | gagcgggtccgcgtgccatgatggtaaggaatggacatatatcggagttgatggccct |
| | | gacaataatgcattgctcaaagtaaaatatggagaagcatatactgacacataccattcc |
| | | tatgcaaacaacatcctaagaacacaagaaagtgcctgcaattgcatcgggggaaatt |
| | | gttatctaatgataactgatggctcagcttcaggtgttagtgaatgcagatttcttaagattc |
| | | gagagggccgaataataaaagaaatatttccaacaggaagagtaaaacacactgagg |
| | | aatgcacatgcggatttgccagcaataaaaccatagaatgtgcctgtagagacaacag |
| | | gtacacagcaaaaagaccttttgtcaaattaaacgtggagactgatacagcagaaataa |
| | | ggttgatgtgcacagatacctatttggacacccccagaccaaatgatggaagcataaca |
| | | ggcccttgtgaatctgatggggacaaagggagtggaggcatcaagggaggatttgttc |
| | | atcaaagaatgaaatccaagattggaaggtggtactctcgaacgatgtctcaaactgaa |
| | | aggatggggatgggactgtatgtcaagtatggtggagacccatgggctgacagtgatg |
| | | ccctagcttttagtggagtaatggtttcaatgaaagaacctggttggtattcctttggcttcg |
| | | aaataaaagataagaaatgcgatgtcccctgtattgggatagagatggtacatgatggt |
| | | ggaaaagagacttggcactcagcagcaacagccatttactgtttaatgggctcaggaca |
| | | gctgctgtgggacactgtcacaggtgttgacatggctctgtaa |
| 62 | B-MPP | MLLVNQSHQGFNKEHTSKMVSAIVLYVLLAAAAHSAFAAD |
| | | PHHHHHHGDHYDDELFSDVQDIKTALAKIHEDNQKIIS |
| | | KLESLLLLKGEVESIKKQINRQNISISTLEGHLSSIMIAIP |
| | | GLSLVPRGSPSRLLLPEPEWTYPRLSCPGSTFQKALLI |
| | | SPHRFGETKGNSAPLIIREPFVACGPNECKHFALTHY |
| | | AAQPGGYYNGTRGDRNKLRHLISVKLGKIPTVENSI |
| | | FHMAAWSGSACHDGKEWTYIGVDGPDNNALLKVK |
| | | YGEAYTDTYHSYANNILRTQESACNCIGGNCYLMIT |
| | | DGSASGVSECRFLKIREGRIIKEIFPTGRVKHTEECT |
| | | CGFASNKTIECACRDNRYTAKRPFVKLNVETDTAEI |
| | | RLMCTDTYLDTPRPNDGSITGPCESDGDKGSGGIKG |
| | | GFVHQRMKSKIGRWYSRTMSQTERMGMGLYVKY |
| | | GGDPWADSDALAFSGVMVSMKEPGWYSFGFEIKDK |
| | | KCDVPCIGIEMVHDGGKETWHSAATAIYCLMGSGQ |
| | | LLWDTVTGVDMAL* |
| | | (signal peptide and his tag are italicized; MPP tetramerization |
| | | domain is underlined; thrombin cleavage site plus two amino |
| | | acid residues is double underline; NA globular domain of |
| | | influenza virus B/Colorado/06/2017 is bold) |

Mouse vaccination and challenge studies. The protective efficacy of recombinant neuraminidase proteins was tested in 6-8 week old female BALB/c mice or DBA2 mice (n=5 per group). The BALB/c mice were used for H1N1 and influenza B virus experiments and the DBA2 mice were used for the H3N2 experiments. The mice were vaccinated intramuscular with 3 μg of the specified recombinant NA protein (in 50 μl PBS). For mice vaccinated with a recombinant NA protein adjuvanted with AddaVax (InvivoGen), a 1:1 ratio of recombinant NA protein and AddaVax was used. An irrelevant protein was administered to one of the groups as negative control and an NA protein, containing a VASP tetramerization domain and the globular head of NA matching the specified recombinant NA protein was included as a positive control, whenever a positive control was used. The mice were vaccinated twice in an interval of 3 weeks. Three or four weeks after the second vaccination, the mice were challenged with indicated dose of the specified virus. Survival as well as the weight loss were monitored over a number of days. Mice that lost more than 25% of their initial body weight were humanely euthanized.

SDS-PAGE. The SDS-PAGE was generally performed as described in Example 1.

ELISA. The ELISAs were generally performed as described in Example 1 using the specified recombinant protein.

NA-Star assay. The NA-Star assay was generally performed as described in Example 1.

NA inhibition assay. For NI assays, microtiter 96-well plates (Immulon 4 HBX; Thermo Fisher Scientific) were coated using a method similar to that employed for the NA assay. The next day, RDE-treated human serum samples were serially diluted 1:2 in separate 96-well plates, with a starting concentration of 1:10. The final volume of diluted serum samples in all wells was 75 μl. Virus stocks were diluted in PBS to the determined EC90 concentration. After virus (75 μl/well) was added to the serum plates, the plates were incubated at room temperature for 1.5 h. The fetuin-coated plates were blocked and then washed under conditions similar to those used for the NA assay. A 100-μl volume of the virus/serum mixture was transferred to the washed fetuin-coated plates, which were then incubated for 2 h at 37° C. The plates were washed four times with PBS-T, and PNA-HRP (Sigma) diluted to 5 μg/ml in PBS (100 μl/well) was added. The NA assay protocol was followed for the remaining NI assay steps. The human serum sample reactivity was determined by subtracting the background absorbance value from the raw absorbance value of human serum samples. The obtained value was divided by the average value from virus-only control wells and then multiplied by a factor of 100 to calculate the NA activity. Percent NI was determined by subtracting NA activity from 100%. Using GraphPad Prism 7, the percent NI was fitted to a nonlinear regression to determine the 50% inhibitory concentration (IC50) of the serum samples. See the website mbio.asm.org/content/8/2/e02281-16.long.

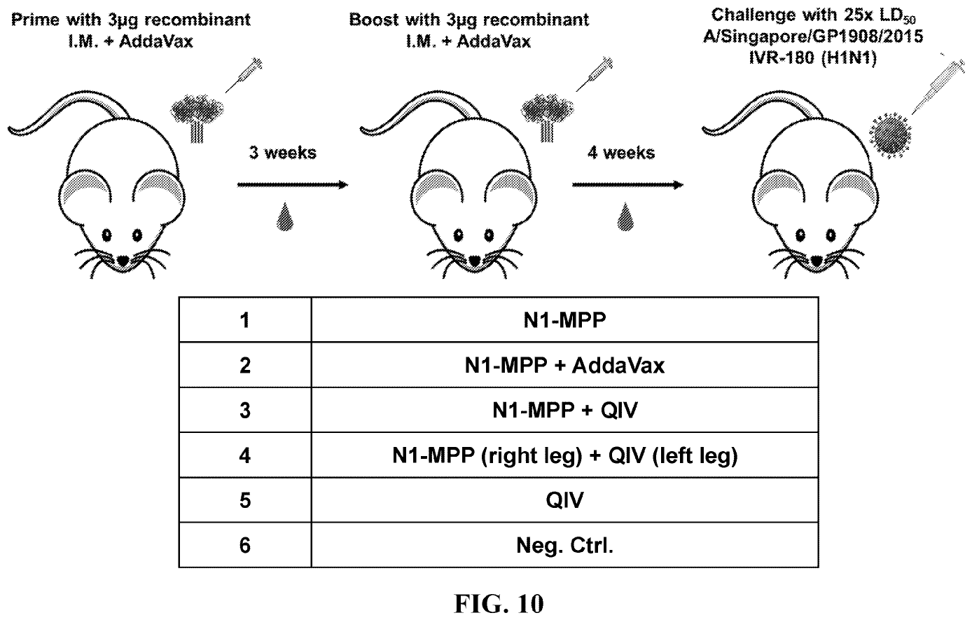

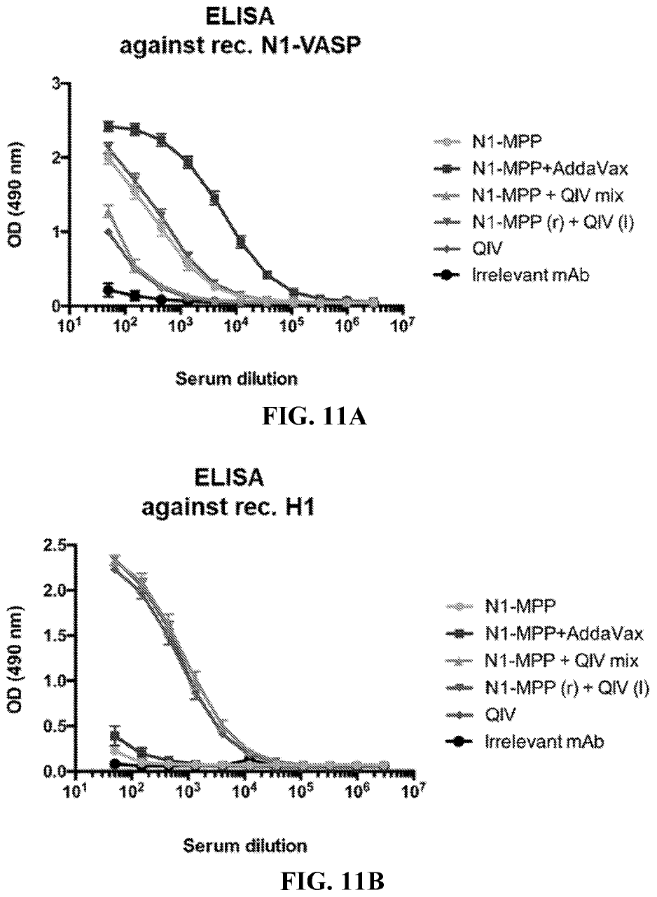

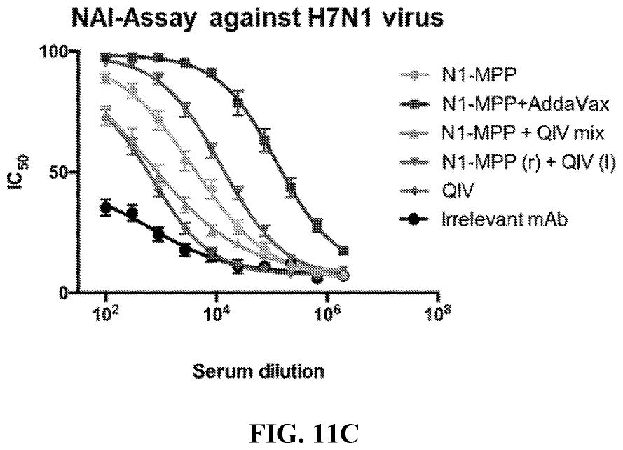

FIG. 11A depicts the results from an ELISA measuring the binding of sera from mice immunized in accordance with the protocol provided in FIG. 10 and the legend for FIG. 10 for groups 1 to 6 to recombinant N1-VASP. The recombinant N1-MPP protein induced good immune responses against NA both when given without adjuvant, with adjuvant or with QIV but in different legs. If given in the same leg with QIV, the anti-NA immune response seems to be dampened. This is not the case with the response to the H1 in the QIV. FIG. 11B depicts the results from an ELISA measuring the binding of sera from mice immunized in accordance with the protocol provided in FIG. 10 and the legend for FIG. 10 for groups 1 to 6 to recombinant H1 to. Sera from mice vaccinated with N1-MPP did not bind to the recombinant H1. FIG. 11C shows the ability of sera from mice immunized in accordance with the protocol provided in FIG. 10 and the legend for FIG. 10 for groups 1 to 6 to inhibit the neuraminidase function of an H7N1 re-assortant virus that contains a matched N1 NA was assessed. The recombinant N1-MPP induced antibodies capable of inhibiting neuraminidase activity.

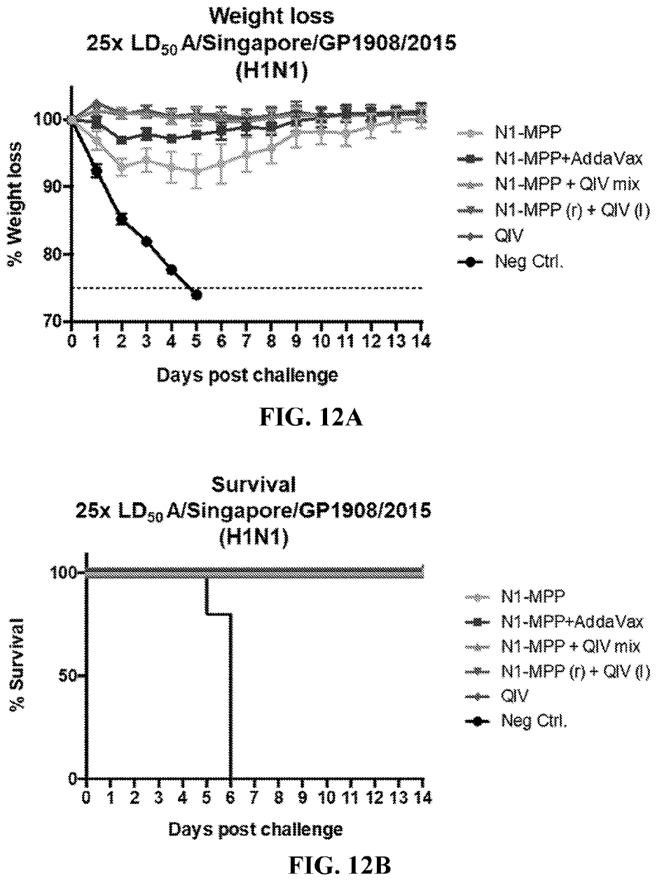

FIG. 12A and FIG. 12B shows that all mice immunized with N1-MPP were protected from weight loss had increased survival following challenge with 25×LD₅₀ of A/Singapore/GP1908/2015 IVR-180 (H1N1), respectively.

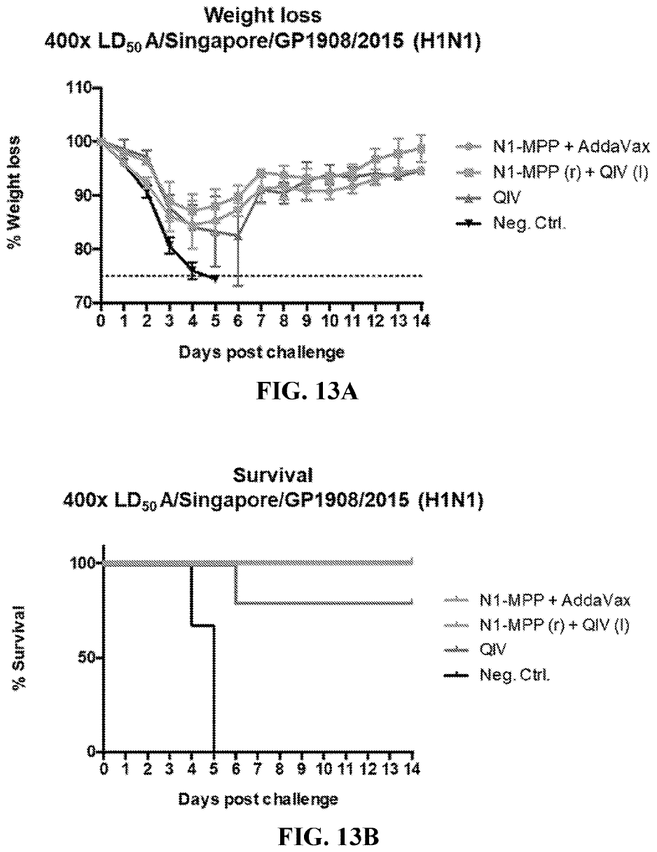

FIG. 13A and FIG. 13B show that all mice immunized with N1-MPP were protected from severe weight loss and had increased survival following challenge with 400×LD₅₀ of A/Singapore/GP1908/2015 IVR-180 (H1N1), respectively.

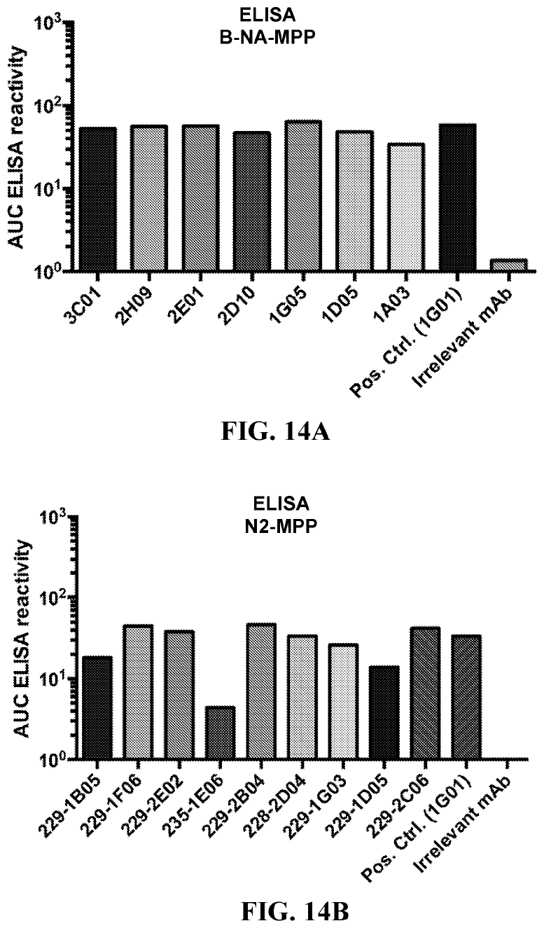

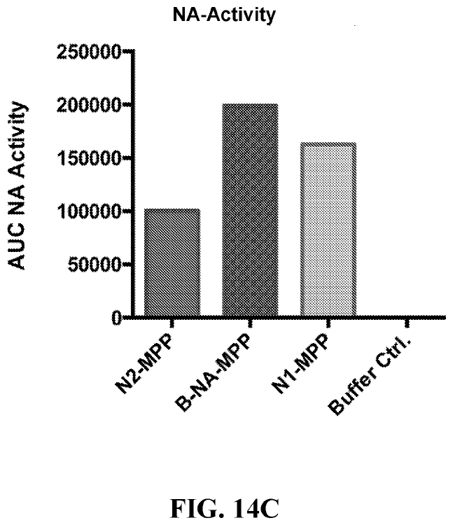

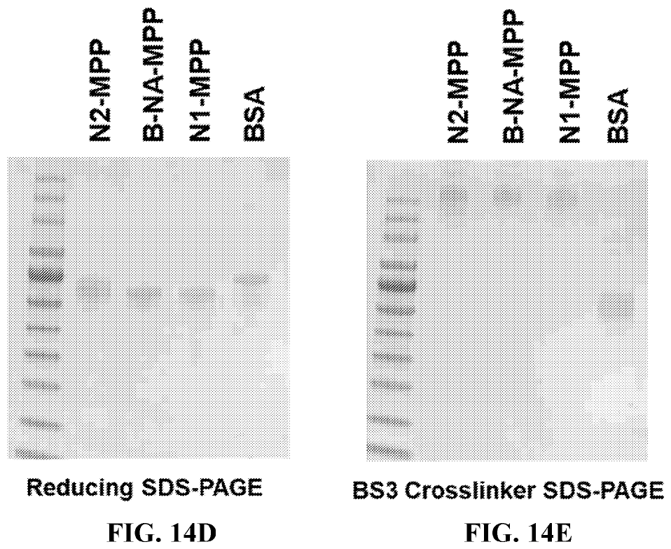

FIG. 14A shows the reactivity of broadly reactive anti-influenza B virus NA monoclonal antibodies to recombinant B-MPP. FIG. 14B shows the reactivity of broadly reactive anti-influenza A virus N2 NA monoclonal antibodies to recombinant N2-MPP. FIG. 14C shows the NA activity of recombinant N1-MPP (SEQ ID NO: 27), recombinant N2-MPP (SEQ ID NO:60), and recombinant B-MPP (SEQ ID NO:62) in an NA-star assay. The NA activity of the recombinant NA proteins indicates that the proteins are correctly folded. FIG. 14D shows recombinant N1-MPP (SEQ ID NO: 27), recombinant N2-MPP (SEQ ID NO:60), and recombinant B-MPP (SEQ ID NO:62) in a reducing SDS-PAGE. FIG. 14E shows recombinant N1-MPP (SEQ ID NO: 27), recombinant N2-MPP (SEQ ID NO: 60), and recombinant B-MPP (SEQ ID NO:62) in a cross-linking SDS-PAGE, which indicates tetramer formation.

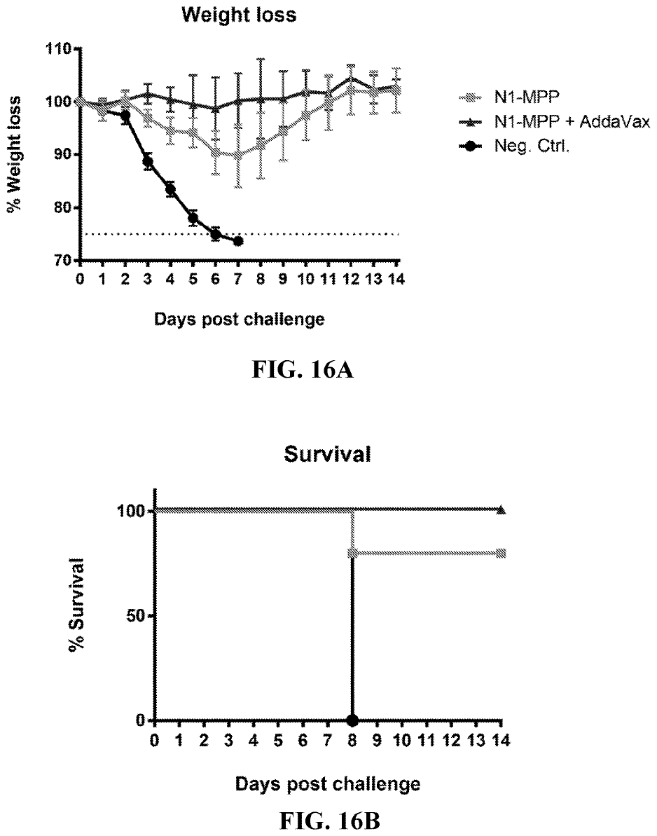

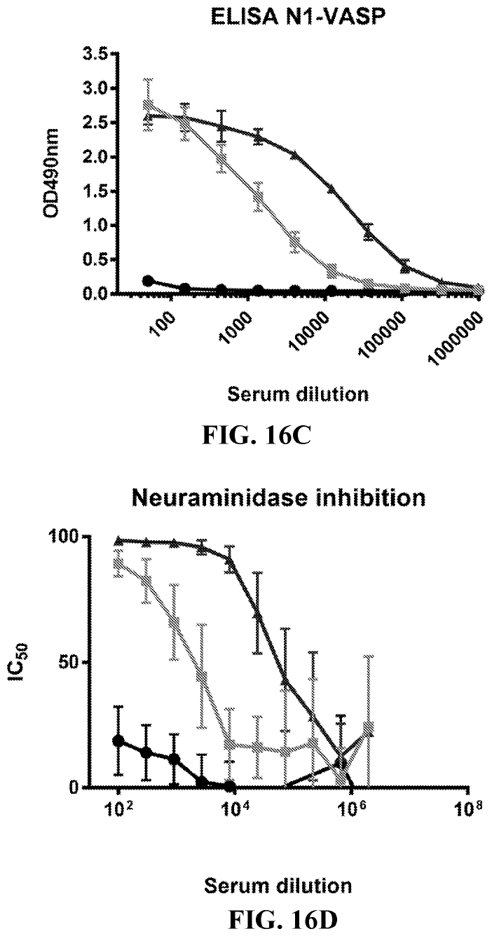

FIG. 16A and FIG. 16B show that all mice immunized with N1-MPP protective mice from weight loss of mice and increased survival following challenge with 25×LD₅₀ of A/Singapore/GP1908/2015 IVR-180 (H1N1), respectively.

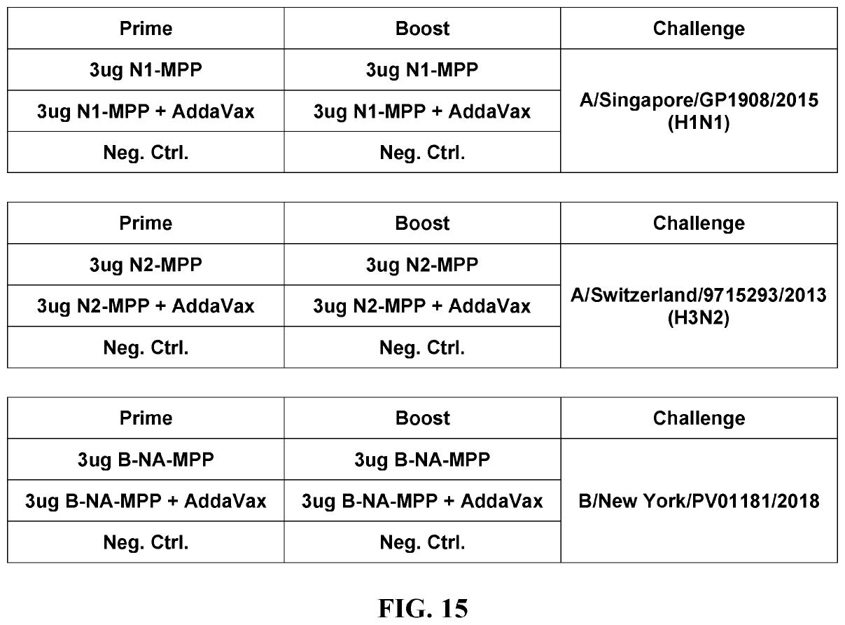

FIG. 16C depicts the results from an ELISA measuring the binding of sera from mice immunized in accordance with the protocol provided in FIG. 15 and the legend for FIG. 15 for groups 1 to 3 to recombinant N1-VASP (which comprises the globular head domain of influenza virus A/Michigan/45/15 NA and the human VASP tetramerization domain). The sera from N1-MPP vaccinated mice is able to bind to N1-VASP.

FIG. 16D depicts the results from functional neuraminidase inhibition assays. Sera from mice vaccinated with N1-MPP as described in FIG. 15 and the legend for FIG. 15 is able to inhibit the neuraminidase function of an H7N1 re-assortant virus that contains a matched N1 NA was assessed.

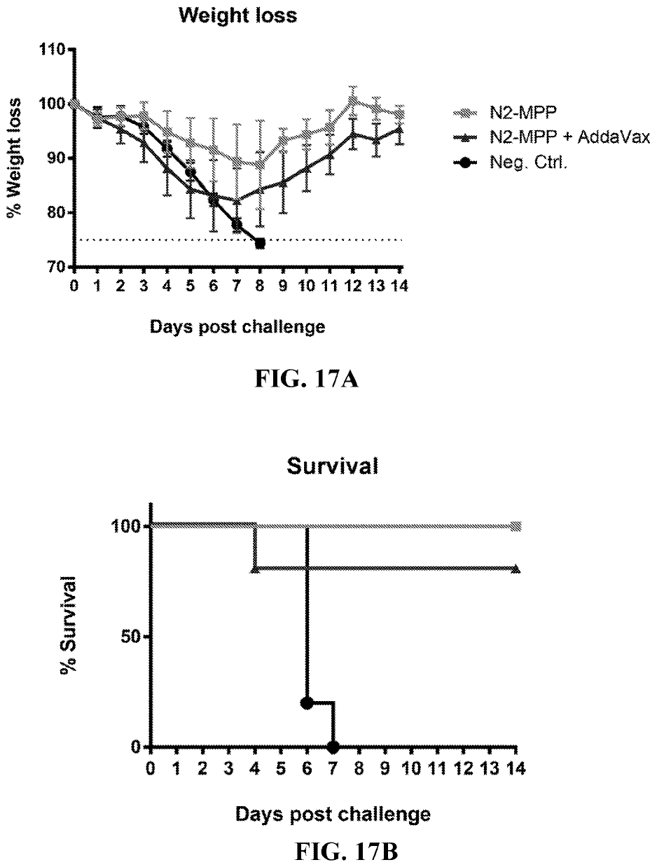

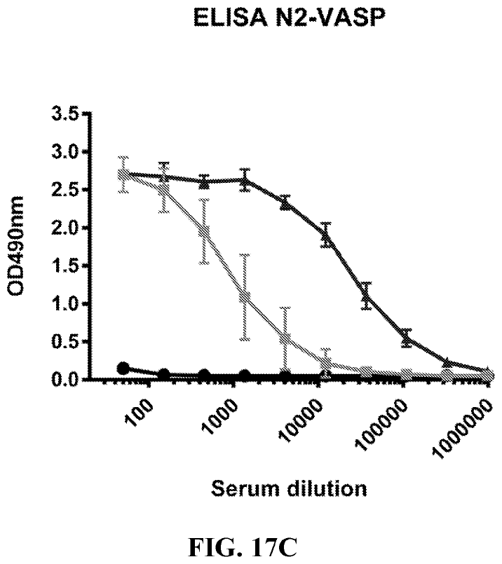

FIG. 17A and FIG. 17B show that all mice immunized with N2-MPP are protected from weight loss and show increased survival following challenge with 25×LD₅₀ of A/Switzerland/9715293/2013 (H3N2, mouse adapted), respectively.

FIG. 17C depicts the results from an ELISA measuring the binding of sera from mice immunized in accordance with the protocol provided in FIG. 15 and the legend for FIG. 15 for groups 1 to 3 to recombinant N2-VASP (which comprises the globular head domain of influenza virus A/Kansas/14/2017 NA and the human VASP tetramerization domain).

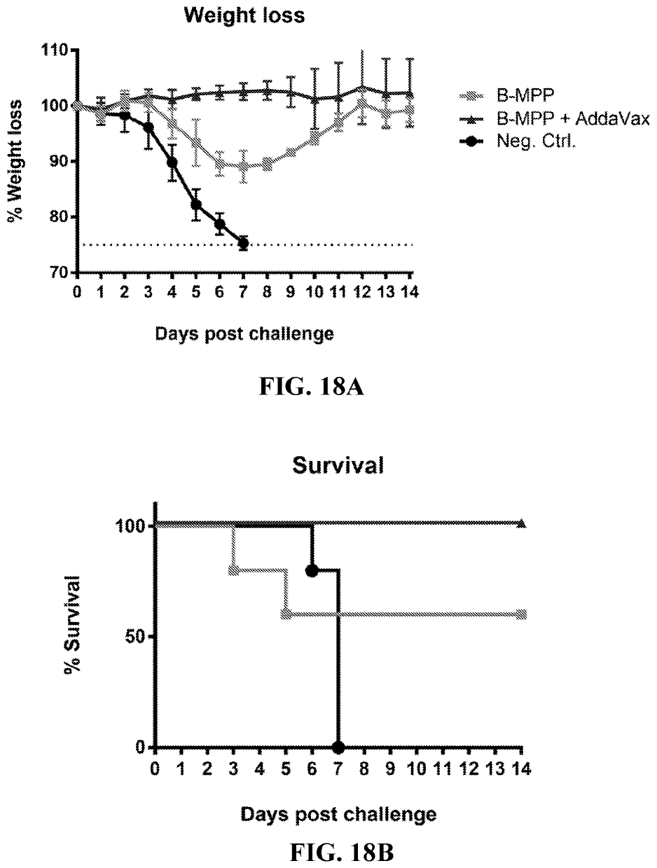

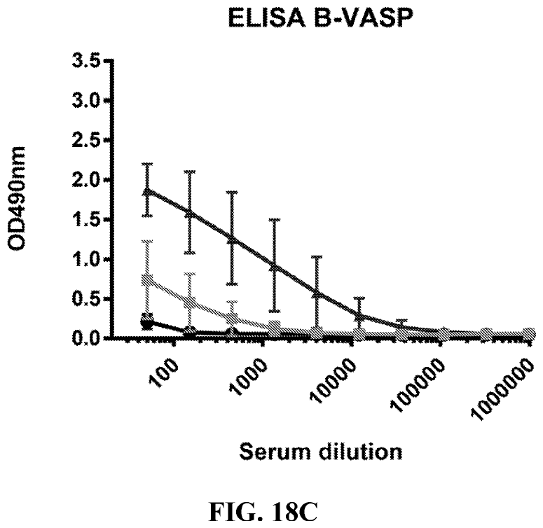

FIG. 18A and FIG. 18B show that all mice immunized with B-MPP are protected from weight loss and show increased survival following challenge with 25×LD₅₀ of B/New York/PV01181/2018, respectively.

FIG. 18C depicts the results from an ELISA measuring the binding of sera from mice immunized in accordance with the protocol provided in the legend for FIG. 15 and FIG. 15 for groups 1 to 3 to recombinant B-VASP (which comprises the globular head domain of influenza virus B/Colorado/06/2017 NA and the human VASP tetramerization domain).

The results described in this example demonstrate that the recombinant N1-MPP protein, recombinant N2-MPP, and recombinant B-MPP are conformationally correct and induce an immune response that is protective in the mouse model against challenge with a virus of the same subtype.

### 7. EMBODIMENTS

1. A recombinant neuraminidase comprising a mutated influenza virus neuraminidase ectodomain, wherein the mutated ectodomain comprises an influenza virus neuraminidase ectodomain comprising amino acid substitutions to cysteine at amino acid residues 48 and 50 of an N1 subtype or at amino acid residues corresponding to amino acid residues 48 and 50 of influenza virus A/Puerto Rico/08/1934.

2. A recombinant neuraminidase comprising a mutated influenza virus neuraminidase ectodomain, wherein the mutated ectodomain comprises an influenza virus neuraminidase ectodomain comprising an amino acid substitution to cysteine at amino acid residue 61 of an N1 subtype or at an amino acid residue corresponding to amino acid residue 61 of influenza virus A/Puerto Rico/08/1934.

3. The recombinant neuraminidase of embodiment 2, wherein the mutated influenza virus neuraminidase ectodomain further comprises an amino acid substitution to a cysteine at amino acid residue 48 of an N1 subtype or at an amino acid residue corresponding to amino acid residue 48 of influenza virus A/Puerto Rico/08/1934.

4. The recombinant neuraminidase of any one of embodiments 1 to 3, wherein the influenza virus neuraminidase ectodomain is of subtype N1 or N2.

5. The recombinant neuraminidase of any one of embodiments 1 to 3, wherein the influenza virus neuraminidase ectodomain is of subtype N3, N4, N5, N6, N7, N8 or N9 subtype.

6. The recombinant neuraminidase of any one of embodiments 1 to 5, wherein the recombinant neuraminidase further comprises the influenza virus neuraminidase transmembrane and cytoplasmic domains.

7. The recombinant neuraminidase of any one of embodiments 1 to 5, wherein the recombinant neuraminidase further comprises a tetramerization domain.

8. The recombinant neuraminidase of embodiment 7, wherein the tetramerization domain comprises a measles virus phosphoprotein tetramerization domain or a Sendai virus phosphoprotein tetramerization domain.

9. A recombinant neuraminidase comprising a mutated influenza virus neuraminidase ectodomain and a tetramerization domain, wherein the mutated ectodomain comprises an influenza virus neuraminidase ectodomain comprising an amino acid substitution to a cysteine at amino acid residue 48 of an N1 subtype or at an amino acid residue corresponding to amino acid residue 48 of influenza virus A/Puerto Rico/08/1934.

10. The recombinant neuraminidase of embodiment 9, wherein the tetramerization domain comprises a measles virus phosphoprotein tetramerization domain or a Sendai virus phosphoprotein tetramerization domain.

11. A recombinant neuraminidase comprising an influenza virus neuraminidase globular head domain and a tetramerization domain of a paramyxovirus protein, wherein the recombinant neuraminidase lacks of influenza virus neuraminidase stalk domain, transmembrane domain and cytoplasmic domain.

12. The recombinant neuraminidase of embodiment 11, wherein the tetramizeration domain is a tetramerization domain of a paramyxovirus phosphoprotein.

13. The recombinant neuraminidase of embodiment 12, wherein the paramyxovirus phosphoprotein is a Nipah virus phosphoprotein, a Hendra virus phosphoprotein, a respiratory syncytial virus phosphoprotein, human parainfluenza virus (hPIV) phosphoprotein, bovine parainfluenza virus phosphoprotein, a mumps virus phosphoprotein, a Cedar virus phosphoprotein, a Ghana virus phosphoprotein, a Newcastle disease virus phosphoprotein, a canine distemper virus phosphoprotein, or a Peste des petits ruminants virus (PPRV) phosphoprotein.

14. The recombinant neuraminidase of embodiment 12, wherein the tetramerization domain is a measles virus phosphoprotein tetramerization domain or a Sendai virus phosphoprotein tetramerization domain.

15. A recombinant neuraminidase comprising a mutated influenza virus neuraminidase ectodomain, wherein the mutated ectodomain comprises an influenza virus neuraminidase ectodomain comprising an amino acid substitution to cysteine at amino acid residue 52 of an N2 subtype or at an amino acid residue corresponding to amino acid residue 52 of influenza virus A/Hong Kong/4801/2014, wherein the ectodomain is from an influenza A virus N2, N3, N4, N5, N6, N7, N8 or N9 subtype.

16. A recombinant neuraminidase comprising a mutated influenza virus neuraminidase ectodomain, wherein the mutated ectodomain comprises an influenza virus neuraminidase ectodomain comprising an amino acid substitution to cysteine at amino acid residue 54 of an N2 subtype or at an amino acid residue corresponding to amino acid residue 54 of A/Hong Kong/4801/2014, wherein the ectodomain is from an influenza A virus N2, N3, N4, N5, N6, N7, N8 or N9 subtype.

17. The recombinant neuraminidase of embodiment 15, wherein the mutated ectodomain further comprises an amino acid substitution to cysteine at amino acid residue 54 of an N2 subtype or at amino acid residue corresponding to amino acid residue 54 of A/Hong Kong/4801/2014.

18. The recombinant neuraminidase of any one of embodiments 15 to 17, wherein the neuraminidase further comprises the influenza virus neuraminidase transmembrane domain and cytoplasmic domain.

19. The recombinant neuraminidase of any one of embodiments 15 to 17, wherein the neuraminidase further comprises a tetramerization domain.

20. The recombinant neuraminidase of embodiment 19, wherein the tetramerization domain comprises a measles virus phosphoprotein tetramerization domain or a Sendai virus phosphoprotein tetramerization domain.

21. The recombinant neuraminidase of any one of embodiments 1 to 20, wherein the recombinant neuraminidase comprises a signal peptide.

22. The recombinant neuraminidase of any one of embodiments 1 to 21, wherein the recombinant neuraminidase further comprises a histidine tag, a Flag tag, or other purification tag.

23. A recombinant neuraminidase comprising the amino acid sequence set forth in SEQ ID NO: 27, 56, 58, 60 or 62.

24. A nucleic acid sequence comprising a nucleotide sequence encoding a recombinant neuraminidase of any one of embodiments 1 to 23.

25. A nucleic acid sequence comprising the nucleotide sequence of SEQ ID NO: 55, 57, 59 or 61.

26. An expression vector comprising the nucleic acid sequence of embodiment 24 or 25.

27. A host cell capable of expressing the nucleic acid sequence of embodiment 24 or 25.

28. A recombinant influenza virus comprising the neuraminidase of any one of embodiments 1 to 8 or 15 to 20.

29. A recombinant influenza virus comprising a genome, wherein the genome comprises a gene segment comprising a nucleic acid sequence encoding the recombinant neuraminidase of any one of embodiments 1 to 8 or 15 to 20 such that the recombinant neuraminidase is expressed by a cell infected with the recombinant influenza virus.

30. A recombinant influenza virus comprising a neuraminidase, wherein the neuraminidase comprises a mutated influenza virus neuraminidase ectodomain, wherein the mutated ectodomain comprises an influenza virus neuraminidase ectodomain comprising an amino acid substitution to cysteine at amino acid residue 48 of an N1 subtype or at an amino acid residue corresponding to amino acid residue 48 of influenza virus A/Puerto Rico/8/1934.

31. A recombinant influenza virus comprising a genome, wherein the genome comprises a gene segment comprising a nucleotide sequence encoding a neuraminidase such that the neuraminidase is expressed by an infected cell, wherein the neuraminidase comprises a mutated influenza virus neuraminidase ectodomain, wherein the mutated ectodomain comprises an influenza virus neuraminidase ectodomain comprising an amino acid substitution to cysteine at amino acid residue 48 of an N1 subtype or at amino acid residue corresponding to amino acid residue 48 of influenza virus A/Puerto Rico/8/1934.

32. A recombinant influenza virus comprising the recombinant neuraminidase of any one of embodiments 9 to 14.

33. A recombinant influenza virus comprising a genome, wherein the genome comprises a gene segment comprising a nucleic acid sequence encoding the recombinant neuraminidase of any one of embodiments 9 to 14 such that the recombinant neuraminidase is expressed by a cell infected by the recombinant influenza virus.

34. The recombinant influenza virus of embodiment 28, 30 or 32, wherein the recombinant influenza virus is inactivated.

35. The recombinant influenza virus of embodiment 28, 30 or 32, wherein the recombinant influenza virus is split.

36. The recombinant influenza virus of any one of embodiments 28 to 35, wherein the recombinant influenza virus is a recombinant influenza A virus.

37. The recombinant influenza virus of any one of embodiments 28 to 33, wherein the recombinant influenza virus is a live attenuated influenza virus.

38. The recombinant influenza virus of embodiment 37, wherein the recombinant influenza A virus is an H1 or H3 subtype.

39. An immunogenic composition comprising the recombinant neuraminidase of any one of embodiments 1 to 23.

40. The immunogenic composition of embodiment 39, wherein the composition further comprises a trivalent inactivated influenza vaccine (TIV), quadrivalent inactivated influenza virus vaccine (QIV), or recombinant influenza virus vaccine.

41. An immunogenic composition comprising the recombinant influenza virus of any one of embodiments 28 to 38.

42. The immunogenic composition of any one of embodiments 39 to 41, wherein the composition further comprises an adjuvant.

43. A method of immunizing against influenza virus, comprising administering a subject a dose of the immunogenic composition of any one of embodiments 39 to 42.

44. A method of inducing an immune response against influenza virus, comprising administering a subject a dose of the immunogenic composition of any one of embodiments 39 to 42.

45. A method of preventing an influenza virus disease, comprising administering to a subject a dose of the immunogenic composition of any one of embodiments 39 to 42.

46. The method of any one of embodiments 43 to 45, wherein the subject is administered a further dose of the immunogenic composition as a boost.

47. The method of any one of embodiments 43 to 46, wherein the subject is human

48. The method of any one of embodiments 43 to 47, wherein the immunogenic composition is administered intramuscularly to the subject.

The foregoing is not to be limited in scope by the specific embodiments described herein. Indeed, various modifications of the antibodies and methods provided herein and their equivalents, in addition to those described herein will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1.-49. (canceled)

50. A method of immunizing against influenza virus or preventing an influenza virus disease, comprising administering to a subject a dose of an immunogenic composition comprising a recombinant neuraminidase, wherein the recombinant neuraminidase comprises:
(a) the amino acid sequence of SEQ ID NO: 56, 58, 27, 60, or 62;
(b) an influenza virus neuraminidase globular head domain and a tetramerization domain, wherein the recombinant neuraminidase lacks an influenza virus neuraminidase stalk domain, transmembrane domain and cytoplasmic domain, wherein the tetramerization domain comprises a paramyxovirus phosphoprotein tetramerization domain, wherein the paramyxovirus phosphoprotein tetramerization domain is a Sendai virus phosphoprotein tetramerization domain that comprises the amino acid sequence of SEQ ID NO: 2; or
(c) the amino acid sequence of SEQ ID NO: 25.

51. A method of inducing an immune response against influenza virus, comprising administering a subject a dose of an immunogenic composition comprising a recombinant neuraminidase, wherein the recombinant neuraminidase comprises:
(a) the amino acid sequence of SEQ ID NO: 56, 58, 27, 60, or 62;
(b) an influenza virus neuraminidase globular head domain and a tetramerization domain, wherein the recombinant neuraminidase lacks an influenza virus neuraminidase stalk domain, transmembrane domain and cytoplasmic domain, wherein the tetramerization domain comprises a paramyxovirus phosphoprotein tetramerization domain, wherein the paramyxovirus phosphoprotein tetramerization domain is a Sendai virus phosphoprotein tetramerization domain that comprises the amino acid sequence of SEQ ID NO: 2; or
(c) the amino acid sequence of SEQ ID NO: 25.

52. (canceled)

53. The method of claim 50, wherein the subject is administered a further dose of the immunogenic composition as a boost.

54. The method of claim 50, wherein the subject is human.

55. The method of claim 50, wherein the immunogenic composition is administered intramuscularly to the subject.

56. The method of claim 51, wherein the subject is administered a further dose of the immunogenic composition as a boost.

57. The method of claim 51, wherein the subject is human.

58. The method of claim 51, wherein the immunogenic composition is administered intramuscularly to the subject.

59. The method of claim 50, wherein the recombinant neuraminidase comprises the amino acid sequence of SEQ ID NO: 27.

60. The method of claim 50, wherein the recombinant neuraminidase comprises the amino acid sequence of SEQ ID NO: 56.

61. The method of claim 50, wherein the recombinant neuraminidase comprises the amino acid sequence of SEQ ID NO: 58.

62. The method of claim 50, wherein the recombinant neuraminidase comprises the amino acid sequence of SEQ ID NO: 60.

63. A method of immunizing against influenza virus or preventing an influenza virus disease, comprising administering to a subject a dose of an immunogenic composition comprising a recombinant influenza virus, wherein the recombinant influenza virus comprises:
(A) the recombinant neuraminidase comprising:
(a) the amino acid sequence of SEQ ID NO: 56, 58, 27, 60, or 62;
(b) an influenza virus neuraminidase globular head domain and a tetramerization domain, wherein the recombinant neuraminidase lacks an influenza virus neuraminidase stalk domain, transmembrane domain and cytoplasmic domain, wherein the tetramerization domain comprises a paramyxovirus phosphoprotein tetramerization domain, and wherein the paramyxovirus phosphoprotein tetramerization domain is a Sendai virus phosphoprotein tetramerization domain that comprises the amino acid sequence of SEQ ID NO: 2; or
(c) the amino acid sequence of SEQ ID NO: 25; and
(B) a genome, wherein the genome comprises a gene segment comprising a nucleic acid sequence encoding the recombinant neuraminidase, such that the recombinant neuraminidase is expressed by a cell infected by the recombinant influenza virus.

64. The method of claim 50, wherein the recombinant neuraminidase comprises the amino acid sequence of SEQ ID NO: 62.

65. The method of claim 50, wherein the amino acid sequence of the recombinant neuraminidase consists of the amino acid sequence of SEQ ID NO: 56, 58, or 27.

66. The method of claim 50, wherein the amino acid sequence of the recombinant neuraminidase consists of the amino acid sequence of SEQ ID NO: 60.

67. The method of claim 50, wherein the amino acid sequence of the recombinant neuraminidase consists of the amino acid sequence of SEQ ID NO: 62.

68. The method of claim 51, wherein the recombinant neuraminidase comprises the amino acid sequence of SEQ ID NO: 56, 58, 27, 60, or 62.

69. The method of claim 63, wherein the recombinant neuraminidase comprises the amino acid sequence of SEQ ID NO: 56, 58, 27, 60, or 62.

70. The method of claim 63, wherein the subject is human.
